(19) United States
(12) Patent Application Publication
(10) Pub. No.: US 2023/0174630 A1
(43) Pub. Date: Jun. 8, 2023

(54) SINGLE CHAIN ANTIBODIES AND INTRABODIES TO MISFOLDED TDP-43 AND METHODS OF USE

(71) Applicants: THE UNIVERSITY OF BRITISH COLUMBIA, Vancouver (CA); PROMIS NEUROSCIENCES INC., Toronto (CA)

(72) Inventors: Neil R. Cashman, Vancouver (CA); Johanne Kaplan, Sherborn, MA (US); Beibei Zhao, Vancouver (CA)

(21) Appl. No.: 17/921,277

(22) PCT Filed: Apr. 29, 2021
(86) PCT No.: PCT/CA2021/050597

§ 371 Date: Oct. 25, 2022

Related U.S. Application Data
(60) Provisional application No. 63/017,363, filed on Apr. 29, 2020.

(51) Int. Cl. C07K 16/18

(52) U.S. Cl. CPC C07K 16/18; C07K 2317/622; C07K 2317/565; C07K 2317/569; C07K 2317/24; C07K 2317/92; C07K 2317/76

(57) ABSTRACT

The disclosure pertains to single chain antibodies, nucleic acids and vectors that encode antibodies that for example specifically bind W68 in the context of DAGWGNL (SEQ ID NO: 1) and methods of administering the single chain antibodies, nucleic acids and vectors to a subject in need thereof.

### RELATED APPLICATIONS

This is a Patent Cooperation Treaty Application which claims priority to U.S. Provisional Patent Application No. 63/017,363, filed on Apr. 29, 2020. The disclosure of the prior application is considered part of the disclosure of this application, and is incorporated in its entirety into this application.

### INCORPORATION OF SEQUENCE LISTING

A computer readable form of the Sequence Listing “P61456PC00 Sequence ListingST25.txt” (154,654 bytes), created on Apr. 29, 2021, is herein incorporated by reference.

### FIELD

The present disclosure relates to TDP-43 single chain antibodies and more specifically to intrabodies for targeting intracellular misfolded TDP-43.

### BACKGROUND

Transactive response (TAR) element DNA binding protein of 43 kDa (TDP-43), is a 414 amino acid protein, and is comprised of an N-terminal ubiquitin like domain (NTD, residues 1-80), two RNA recognition motifs (RRMs) composed of residues 106-177 (RRM1), and residues 192-259 (RRM2), and a C-terminal domain (CTD, residues 274-414). The NTD flanks a domain that directs nuclear localization (NLS motifs in residues 82-98, NLS1 K82RK84 and K95VKR98). RRM2 includes a nuclear export signal (NES) from residue 239 to 250.

TDP-43 is predominantly a nuclear protein that plays a central role in RNA metabolism. TDP-43 has become a focal point of research in the amyotrophic lateral sclerosis (ALS) and frontotemporal dementia (FTD) disease spectrum, since pathogenic inclusions within affected neurons can contain post-translationally modified TDP-43. The CTD of TDP-43 is particularly relevant to disease, as it is where nearly all familial ALS/FTD-associated mutations are found in TDP-43.

TDP-43 was found to be hyperphosphorylated, ubiquitinated, and fragmented in neuronal inclusions of patients with both sporadic and familial forms of ALS and FTD [4].

Functional TDP-43 can exist as nuclear oligomers that are distinct from cytoplasmic aggregates formed upon cellular stress. Functional TDP-43 oligomerization is required for its RNA-splicing function. NTD-driven TDP-43 oligomerization in the nucleus can inhibit cytoplasmic mislocalization and the formation of pathologic aggregation [9].

Physiological TDP-43 oligomerization is mediated by its N-terminal domain, which can adopt dynamic, solenoid-like structures, revealed by a 2.1 A crystal structure in combination nuclear magnetic resonance spectroscopy and electron microscopy [9].

Aggregates (inclusion bodies) of TDP-43 have now been found in nearly all (approx. 97%) cases of ALS and roughly half (approx. 40%) of the cases of FTD. TDP-43 is one of the main components of the cytoplasmic inclusions found in the motor neurons and glial cells of ALS patients.

Precursers of TDP-43 inclusions may have concentration far below that of functional TDP-43. The low concentration of misfolded TDP-43 makes this target elusive.

Intracerebral injections of brain derived pathological TDP-43 FTLD-TDP seeds in transgenic mice expressing cytoplasmic human TDP-43 and non-transgenic mice, and has led to the induction of de novo TDP-43 pathology which spreads through the brain in a time dependent manner [10].

Antibodies that bind TDP-43 have been described.

WO2012174666 titled METHODS FOR THE PROGNOSTIC AND/OR DIAGNOSTIC OF NEURODEGENERATIVE DISEASE, METHODS TO IDENTIFY CANDIDATE COMPOUNDS AND COMPOUNDS FOR TREATING NEURODEGENERATIVE DISEASE discloses methods for diagnosing neurodegenerative diseases such as ALS and FTD through assessing the interaction between TDP-43 and NF-κB p65 using an anti-TDP-43 antibody.

WO2016086320 titled TDP-43-BINDING POLYPEPTIDES USEFUL FOR THE TREATMENT OF NEURODEGENERATIVE DISEASES discloses antibodies that bind to the RRM1 domain of TDP-43 to disrupt its interaction with NF-κB for the treatment of ALS and FTD.

Antibodies that can be expressed intracellularly and preferentially bind misfolded TDP-43 over natively folded TDP-43 are desirable.

### SUMMARY

The inventors have identified single chain intrabodies that can target cytoplasmic misfolded TDP-43 and that can for example increase its degradation when expressed in cells comprising cytoplasmic misfolded TDP-43.

The single chain intrabodies are derived from antibodies that bind a conformational N-terminal epitope that is accessible in misfolded TDP-43 but is unavailable in natively folded non-disease associated TDP-43. Antibodies raised to an immunogen comprising the N-terminal TDP-43 sequence DAGWGNL (SEQ ID NO: 1), preferentially bound misfolded TDP-43 aggregates. Residue W68 was found to be an important residue in conferring antibody specificity for misfolded TDP-43 aggregates.

An aspect includes a single chain antibody that binds misfolded TDP-43 and comprises a heavy chain variable region comprising complementarity determining regions CDR-H1, CDR-H2 and CDR-H3 and a light chain variable region comprising complementarity determining regions CDR-L1, CDR-L2 and CDR-L3, wherein the heavy chain variable region and the light chain variable region are linked by a linker. The orientation of the heavy and light chain variable regions and linker can be heavy chain variable region—linker—light chain variable region or light chain variable region—linker—heavy chain variable region.

In an embodiment, the single chain antibody is a scFv, nanobody or minibody.

A further aspect comprises an immunoconjugate comprising an antibody described herein and a detectable label, such as a positron emitting radionuclide a fusion tag, such as a FLAG tag or myc tag, or a targeting moiety such as a lysosomal or autophagy targeting sequence.

A further aspect comprises an isolated nucleic acid encoding the single chain antibody described herein, as well as vectors comprising the nucleic acid, for example, for delivering and/or expressing the single chain antibody described herein.

A further aspect comprises a cell recombinantly expressing a single chain antibody described herein.

A further aspect includes a composition comprising the single chain antibody, immunoconjugate, isolated nucleic acid, vector or a cell described herein.

Further provided is a method of treating a subject with a TDP-43 proteinopathy, the method comprising administering to a subject in need thereof an effective amount of a nucleic acid encoding the single chain antibody or immunoconjugate, described herein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 shows immunocytochemistry of cells overexpressing dNLS-TDP43 and LYS-2F7. dNLS-TDP43 was detected using anti-HA, and LYS-2F7 was detected using anti-FLAG. The merge shows levels of co-localization in reference with the nucleus stained with DAPI;

FIG. 2 shows immunocytochemistry of cells overexpressing dNLS-TDP43 and LYS-1H3-1K3. dNLS-TDP43 was detected using anti-HA, and LYS-1H3-1K3 was detected using anti-FLAG. The merge shows levels of co-localization in reference with the nucleus stained with DAPI;

FIG. 3 shows immunocytochemistry of cells overexpressing dNLS-TDP43 and LYS-28H3-28K1. dNLS-TDP43 was detected using anti-HA, and LYS-28H3-28K1 was detected using anti-FLAG. The merge shows levels of co-localization in reference with the nucleus stained with DAPI;

FIG. 4 shows immunocytochemistry of cells overexpressing dNLS-TDP43 and LYS-14H1-14K2. dNLS-TDP43 was detected using anti-HA, and LYS-14H1-14K2 was detected using anti-FLAG. The merge shows levels of co-localization in reference with the nucleus stained with DAPI;

FIG. 5 shows immunocytochemistry of cells overexpressing dNLS-TDP43 and YPTL-2F7. dNLS-TDP43 was detected using anti-HA, and YPTL-2F7 was detected using anti-FLAG. The merge shows levels of co-localization in reference with the nucleus stained with DAPI;

FIG. 6 shows immunocytochemistry of cells overexpressing dNLS-TDP43 and YPTL-1H3-1K3. dNLS-TDP43 was detected using anti-HA, and YPTL-1H3-1K3 was detected using anti-FLAG. The merge shows levels of co-localization in reference with the nucleus stained with DAPI;

FIG. 7 shows immunocytochemistry of cells overexpressing dNLS-TDP43 and YPTL-28H3-28K1. dNLS-TDP43 was detected using anti-HA, and YPTL-28H3-28K1 was detected using anti-FLAG. The merge shows levels of co-localization in reference with the nucleus stained with DAPI;

FIG. 8 shows immunocytochemistry of cells overexpressing dNLS-TDP43 and YPTL-14H1-14K2. dNLS-TDP43 was detected using anti-HA, and YPTL-14H1-14K2 was detected using anti-FLAG. The merge shows levels of co-localization in reference with the nucleus stained with DAPI;

FIG. 9 shows immunocytochemistry of cells overexpressing dNLS-TDP43 and MYCL15H-2F7. dNLS-TDP43 was detected using anti-HA, and MYCL15H-2F7 was detected using anti-MYC. The merge shows levels of co-localization in reference with the nucleus stained with DAPI;

FIG. 10 shows immunocytochemistry of cells overexpressing dNLS-TDP43 and MYCH15L-2F7. dNLS-TDP43 was detected using anti-HA, and MYCH15L-2F7 was detected using anti-MYC. The merge shows levels of co-localization in reference with the nucleus stained with DAPI;

FIG. 11 shows immunocytochemistry of cells overexpressing dNLS-TDP43 and MYCH20L-2F7. dNLS-TDP43 was detected using anti-HA, and MYCH20L-2F7 was detected using anti-MYC. The merge shows levels of co-localization in reference with the nucleus stained with DAPI;

FIG. 12 shows immunocytochemistry of cells overexpressing dNLS-TDP43 and MYCL15H-1H3-1K3. dNLS-TDP43 was detected using anti-HA, and MYCL15H-1H3-1K3 was detected using anti-MYC. The merge shows levels of co-localization in reference with the nucleus stained with DAPI;

FIG. 13 shows immunocytochemistry of cells overexpressing dNLS-TDP43 and MYCL20H-1H3-1K3. dNLS-TDP43 was detected using anti-HA, and MYCL20H-1H3-1K3 was detected using anti-MYC. The merge shows levels of co-localization in reference with the nucleus stained with DAPI;

FIG. 14 shows immunocytochemistry of cells overexpressing dNLS-TDP43 and MYCH15L-28H3-28K1. dNLS-TDP43 was detected using anti-HA, and MYCH15L-28H3-28K1 was detected using anti-MYC. The merge shows levels of co-localization in reference with the nucleus stained with DAPI;

FIG. 15 shows immunocytochemistry of cells overexpressing dNLS-TDP43 and MYCL15H-14H1-14K2. dNLS-TDP43 was detected using anti-HA, and MYCL15H-14H1-14K2 was detected using anti-MYC. The merge shows levels of co-localization in reference with the nucleus stained with DAPI;

FIG. 16 shows immunocytochemistry of cells overexpressing dNLS-TDP43 and MYCH15L-14H1-14K2. dNLS-TDP43 was detected using anti-HA, and MYCH15L-14H1-14K2 was detected using anti-MYC. The merge shows levels of co-localization in reference with the nucleus stained with DAPI;

FIG. 17 shows immunocytochemistry of cells overexpressing dNLS-TDP43 and MYCL20H-14H1-14K2. dNLS-TDP43 was detected using anti-HA, and MYCL20H-14H1-14K2 was detected using anti-MYC. The merge shows levels of co-localization in reference with the nucleus stained with DAPI;

FIG. 18 shows immunocytochemistry of cells overexpressing dNLS-TDP43 and MYCH20L-14H1-14K2. dNLS-TDP43 was detected using anti-HA, and MYCH20L-14H1-14K2 was detected using anti-MYC. The merge shows levels of co-localization in reference with the nucleus stained with DAPI;

FIG. 19 shows a western blot of cells overexpressing dNLS-TDP43 and LYS-2F7, LYS-28H3-28K1, or LYS-14H1-14K2. Anti-HA was used to detect expression levels of dNLS-TDP43. Anti-FLAG was used to detect expression levels of LYS-2F7, LYS-28H3-28K1, and LYS-14H1-14K2. Actin was used as a loading control. EV (empty vector) is a reference negative control plasmid;

FIG. 20 shows a western blot of cells overexpressing dNLS-TDP43 and YPTL-14H1-14K2. Anti-HA was used to detect expression levels of dNLS-TDP43. Anti-FLAG was used to detect expression levels of YPTL-14H1-14K2. Actin was used as a loading control. EV is a reference negative control;

FIG. 21 shows a western blot of cells overexpressing dNLS-TDP43 and MYCL15H-2F7, MYCH15L-2F7, MYCL15H-1H3-1K3, or MYCH20L-14H1-14K2. Anti-HA was used to detect expression levels of dNLS-TDP43. Anti-MYC was used to detect expression levels of MYCL15H-2F7, MYCH15L-2F7, MYCL15H-1H3-1K3, and MYCH20L-14H1-14K2. Actin was used as a loading control. EV is a reference negative control.

### DETAILED DESCRIPTION OF THE DISCLOSURE

### I. Definitions

As used herein, the term “TDP-43” (transactivation response element (TAR) DNA-binding protein 43) alternately referred to as “TDP43”, or “TDP” unless otherwise qualified, as used herein means all forms of TDP-43 including wild type TDP-43, native TDP-43, as well as misfolded forms including mutant forms and analogs thereof from all species, particularly human TDP-43 (i.e. hTDP-43). Human TDP-43 is a protein of typically 414 amino acid residues and the amino acid sequence (e.g. Uniprot Accession number Q13148) and the nucleotide sequence (e.g. Accession number HGNC:11571) have been previously characterized.

“Wild type” as used herein refers to the primary amino acid sequence of non-mutant or naturally occurring protein.

“Native” as used herein refers to the normal three dimensional structure of a specific protein or part thereof). Native TDP-43 is optionally referred to as “natively folded” TDP-43 “normally folded” TDP-43 and/or “healthy” TDP-43. Accordingly the term “native TDP-43”, or “natively folded TDP-43”, herein refers to TDP-43 as natively folded after nascent translation and/or multimers including but not limited to dimeric TDP-43 and trimeric TDP-43, as folded in non-disease states (e.g. healthy cells) with a molecular structure that comprises a non-covalently associated, individual TDP-43 peptide which shows native structure under in x-ray crystallography or as reconstructed from nuclear magnetic resonance spectra. Native TDP-43 forms multimers through its NTD and TDP-43 when natively folded is typically nuclear. Misfolded aggregates of TDP-43 can be and are typically cytoplasmic.

“Misfolded” as used herein refers to the secondary and tertiary structure of a polypeptide or part thereof, and indicates that the polypeptide has adopted a conformation that is not normal for that polypeptide in its properly functioning state. Although misfolding can be caused by mutations in a protein, such as amino acid deletion, substitution, or addition, wild-type sequence protein can also be misfolded in disease, and expose disease-specific epitopes for instance, as a result of microenvironmental conditions and/or amino acid modification such as nitration, oxidation, carbonylation or other modification. Other post-translational modifications include aberrant ubiquitination, phosphorylation, acetylation, sumoylation, and cleavage into C-terminal fragments. Misfolded TDP43 can be aggregated and/or cytosolic. In the context of TDP-43, native TDP-43 forms multimers through its NTD. Misfolded multimers (e.g. disease-associated oligomers) typically oligomerize through other regions of the protein, for example its LCD and/or RRM1 domains. Accordingly, “misfolded TDP-43 polypeptide”, or “misfolded TDP-43” when referring to the polypeptide herein includes TDP-43 polypeptide that is oligomerized through its LCD and/or RRM1 domains, non-native dimers and trimers, as well as larger aggregates (e.g. 5 or greater subunits), which is cytosolic and/or is aggregated. Misfolded TDP-43 is prone to the formation of aggregates which results in a loss of protein function, toxicity, possession of amyloid-like features (e.g. congo red staining) and propagation of pathogenic aggregates.

The term “mutant TDP-43” refers to forms of TDP-43, and particularly endogenous forms of TDP-43 that occur as a result of genetic mutation that result for instance in amino acid substitution, such as those substitutions characteristic for instance of FTD or familial ALS including for example the mutations described in the bioinformatics tool described in [6].

The term “DAGWGNL (SEQ ID NO: 1)” means the amino acid sequence: aspartic acid, alanine, glycine, tryptophan, glycine, asparagine, and leucine as shown in SEQ ID NO: 1. Similarly GWG refers to the amino acid sequences identified by the 1-letter amino acid code. Depending on the context, the reference of the amino acid sequence can refer to a sequence in TDP-43 or an isolated peptide. The sequence DAGWGNL (SEQ ID NO: 1) corresponds to residues 65-71 in the amino acid primary sequence of TDP-43.

The term “amino acid” includes all of the naturally occurring amino acids as well as modified L-amino acids as well as D-amino acids. The atoms of the amino acid can for example include different isotopes. For example, the amino acids can comprise deuterium substituted for hydrogen, nitrogen-15 substituted for nitrogen-14, and carbon-13 substituted for carbon-12 and other similar changes.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-group size for one another. Examples of conservative amino acid substitution include:

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

The term “antibody” as used herein is intended to include monoclonal antibodies, polyclonal antibodies, single chain, humanized and other chimeric antibodies, or fully human antibodies, as well as binding fragments thereof. Also included are vectorized antibodies or intrabodies. The antibody may be from recombinant sources and/or produced in transgenic animals. Also included are human antibodies that can be produced through using biochemical techniques or isolated from a library. Humanized or chimeric antibody may include sequences from one or more than one isotype or class.

The phrase “isolated antibody” refers to antibody produced in vivo or in vitro that has been removed from the source that produced the antibody, for example, an animal, hybridoma or other cell line (such as recombinant cells that produce antibody). The isolated antibody is optionally “purified”, which means at least: 80%, 85%, 90%, 95%, 98% or 99% purity.

The term “intrabody” or “intrabodies” as used herein refers to an antibody that is expressed or can be expressed in a cell and that binds to an intracellular protein, for example an intrabody is an antibody that has been modified or adapted for intracellular localization and intracellular function. An intrabody comprises a heavy chain variable domain and a light chain variable domain and linker optionally in either variable domain orientation, e.g. heavy chain variable domain-linker—light chain variable domain or light chain variable domain-linker—heavy chain variable domain. Depending on the context, the term intrabody may refer to a nucleic acid molecule or a polypeptide molecule.

The term “linker” as used herein refers to a synthetic sequence (e.g, amino acid sequence in a polypeptide or nucleic acid sequence in a nucleic acid) that connects or links two sequences, e.g, that link two polypeptide domains. The linker can be a “tag linker” indicating that it is linking a detectable label or a targeting moiety linker indicating that it is linking a targeting moiety to a polypeptide which may also comprise a linker as in the case of a heavy chain variable region linked to a light chain variable region.

The term “complementarity determining region” or “CDR” as used herein refers to particular hypervariable regions of antibodies that are commonly understood to define epitope binding. Computational methods for identifying CDR sequences include Kabat, Chothia, and IMGT. A person skilled in the art having regard to the sequences comprised herein would also be able to identify CDR sequences based on Kabat and Chothia etc.

The term “detectable label” as used herein refers to moieties such as peptide sequences, fluorescent proteins that can be appended or introduced into a peptide, antibody or other compound described herein and which is capable of producing, either directly or indirectly, a detectable signal.

The term “epitope selectively presented or accessible in misfolded TDP-43” as used herein refers to an epitope that is selectively presented or antibody-accessible on misfolded TDP-43 as present for example in ALS or FTD (e.g. disease associated misfolded TDP-43) whether in monomeric, dimeric or aggregated forms, but not on the molecular surface of the native, correctly folded, homodimeric form of TDP-43. As shown herein, W68 is selectively presented or accessible in misfolded TDP-43.

The term “greater affinity” as used herein refers to a degree of antibody binding where an antibody X binds to target Y more strongly (Kₒₙ) and/or with a smaller dissociation constant (K_{off}) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as KA equal to 1/K_{D} where K_{D} is equal to kₒₙ/k_{off}. The kₒₙ and k_{off} values can be measured using surface plasmon resonance (measurable for example using a Biacore system).

The term “nucleic acid sequence” as used herein refers to a sequence of nucleotide or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid (DNA) sequences or ribonucleic acid (RNA) sequences and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences as well as codon optimized or synonymous codon equivalents. The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived.

“Operatively linked” is intended to mean that the nucleic acid is linked to regulatory sequences in a manner which allows expression of the nucleic acid. Suitable regulatory sequences may be derived from a variety of sources, including bacterial, fungal, viral, mammalian, or insect genes. Selection of appropriate regulatory sequences is dependent on the host cell chosen and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, including lentiviral vectors, Adeno Associated viral (AAV) vectors and the like. The term “plasmid” as used herein generally refers to a construct of extrachromosomal genetic material, usually a circular DNA duplex, which can replicate independently of chromosomal DNA.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5×sodium chloride/sodium citrate (SSC)/5×Denhardt's solution/1.0% SDS at Tm—5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

As used herein “binds” or “specifically binds” in reference to an antibody means that the antibody recognizes its target antigen and binds its target with greater affinity than it does to a structurally different antigen and/or to an antigen with modified or mutated sequence. For example a multivalent antibody binds its target with K_{D} of at least 1e-6, at least 1e-7, at least 1e-8, at least 1e-9 or at least 1e-10. Affinities greater than at least 1e-8 are preferred. An antigen binding fragment such as Fab fragment comprising one variable domain, may find its target with a 10 fold or 100 fold less affinity than a multivalent interaction with a non-fragmented antibody.

The term “selective” or “preferential” as used herein with respect to an antibody that selectively/preferentially binds a form of TDP-43 (e.g. native, or misfolded protein) means that the binding protein binds the form with at least 3 fold, or at least 5 fold, at least 10 fold, at least 20 fold, at least 100 fold, at least 250 fold, or at least 500 fold or more greater affinity. Accordingly an antibody that is more selective for a particular conformation (e.g. misfolded protein) preferentially binds the particular form of TDP-43 with at least 3 fold, or at least 5 fold, at least 10 fold, at least 20 fold, at least 100 fold, at least 250 fold, or at least 500 fold or more greater affinity compared to another form.

The term “animal” or “subject” as used herein includes all members of the animal kingdom including mammals, optionally including or excluding humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment, for example in a subject identified as carrying a mutation associated with familial forms, such as the familial form of ALS. A subject with a TDP-43 proteinopathy such as ALS can be treated to delay or slow disease progression. Subjects can be treated with a compound, antibody (including vectorized antibody or intrabody), immunogen, immunoconjugate, or composition described herein to prevent progression.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a”, “an” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Antibodies, Immunoconjugates, Cells and Nucleic Acids

As described in the Examples, single chain antibodies were prepared and vectorized.

The single chain antibodies are directed to an N-terminal epitope that is accessible in misfolded TDP-43 but is unavailable in natively folded non-disease associated TDP-43. Antibodies raised to an immunogen comprising the N-terminal TDP-43 sequence DAGWGNL (SEQ ID NO: 1), preferentially bound misfolded TDP-43 aggregates. Residue W68 was found to be an important residue in conferring antibody specificity for misfolded TDP-43 aggregates.

In one embodiment, the single chain antibodies are intrabodies.

The single chain antibodies, and particularly when as intrabodies, optionally include a lysosomal-targeting or autophagy-targeting signal. The heavy chain and light chain variable regions of several antibodies specific for misfolded TDP-43 were linked using various linkers and in various orientations. The vectorized single chain antibodies or intrabodies, were expressed in cells along with mutant dNLS-TDP-43 which comprises deletion of its nuclear localization signal. dNLS-TDP-43 localizes to the cytoplasm where it forms aggregates. As demonstrated herein, the vectorized antibodies were able to co-localize with and induce degradation of intracellular misfolded TDP-43 aggregates. The vectorized antibodies were not toxic to the cells confirming their lack of interference with normal TDP-43 function.

An aspect includes a single chain antibody that binds W68 in misfolded TDP-43 and comprises a heavy chain variable region comprising complementarity determining regions CDR-H1, CDR-H2 and CDR-H3 and a light chain variable region comprising complementarity determining regions CDR-L1, CDR-L2 and CDR-L3, wherein the heavy chain variable region and the light chain variable region are linked by a linker. The orientation of the heavy and light chain variable regions and linker can be heavy chain variable region—linker-light chain variable region or light chain variable region—linker—heavy chain variable region. In some embodiments, the single chain antibody further comprises a lysosomal or autophagy targeting sequence.

In one embodiment, the single chain antibody has CDR sequences comprising

SEQ ID NO: 130
CDR-H1: GFTFSSYY;
SEQ ID NO: 131
CDR-H2: INSNGGST;
SEQ ID NO: 132
CDR-H3: VRQNYEGAY;
SEQ ID NO: 133
CDR-L1: QSIVHSNGNTY;
SEQ ID NO: 134
CDR-L2: KVS;
and
SEQ ID NO: 135
CDR-L3: FQSSHVPWT.

Single chain antibodies comprising CDRs SEQ ID NO: 130-135, specifically bind W68 in the context of DAGWGNL (SEQ ID NO: 1) in misfolded TDP-43. The single chain antibody comprises CDR sequences of antibody 2F7.

In an embodiment, the single chain antibody comprises a heavy chain variable region comprising: i) an amino acid sequence as set forth in SEQ ID NO: 138, ii) an amino acid sequence with at least 80%, at least 85%, at least 90% or at least 95% sequence identity to SEQ ID NO: 138, wherein the CDR sequences are as set forth in SEQ ID NOs: 130-132, or iii) a conservatively substituted amino acid sequence of i), and/or wherein the single chain antibody comprises a light chain variable region comprising an amino acid sequence as set forth in SEQ ID NO: 139, ii) an amino acid sequence with at least 80%, at least 85%, at least 90% or at least 95% sequence identity to SEQ ID NO: 139, wherein the CDR sequences are as set forth in SEQ ID NOs: 133-135, or iii) a conservatively substituted amino acid sequence of i), optionally wherein the heavy chain variable region amino acid sequence is encoded by a nucleotide sequence as set out in SEQ ID NO: 136 or a codon degenerate or optimized version thereof and/or the light chain variable region amino acid sequence is encoded by a nucleotide sequence as set out in SEQ ID NO: 137 or a codon degenerate or optimized version thereof.

In one embodiment, the single chain antibody has CDR sequences comprising

SEQ ID NO: 10
CDR-H1: GFSLSRYY;
SEQ ID NO: 11
CDR-H2: IIPGGTT;
SEQ ID NO: 12
CDR-H3: AGGPTGNSHFTL;
SEQ ID NO: 13
CDR-L1: ESVYNNNH;
SEQ ID NO: 14
CDR-L2: EAS;
and
SEQ ID NO: 15
CDR-L3: SGYKRVTTDGIA.

Single chain antibodies comprising CDRs SEQ ID NO: 10-15, specifically bind W68 in the context of DAGWGNL (SEQ ID NO: 1) in misfolded TDP-43. The single chain antibody comprises CDR sequences of antibody 1H3-1 K3.

In an embodiment, the single chain antibody comprises a heavy chain variable region comprising: i) an amino acid sequence as set forth in SEQ ID NO: 98, ii) an amino acid sequence with at least 80%, at least 85%, at least 90% or at least 95% sequence identity to SEQ ID NO: 98, wherein the CDR sequences are as set forth in SEQ ID NOs: 10-12, or iii) a conservatively substituted amino acid sequence of i), and/or wherein the single chain antibody comprises a light chain variable region comprising an amino acid sequence as set forth in SEQ ID NO: 99, ii) an amino acid sequence with at least 80%, at least 85%, at least 90% or at least 95% sequence identity to SEQ ID NO: 99, wherein the CDR sequences are as set forth in SEQ ID NOs: 13-15, or iii) a conservatively substituted amino acid sequence of i), optionally wherein the heavy chain variable region amino acid sequence is encoded by a nucleotide sequence as set out in SEQ ID NO: 76 or a codon degenerate or optimized version thereof and/or the light chain variable region amino acid sequence is encoded by a nucleotide sequence as set out in SEQ ID NO: 77 or a codon degenerate or optimized version thereof.

In one embodiment, the single chain antibody has CDR sequences comprising

SEQ ID NO: 120
CDR-H1: GFSLSSYN;
SEQ ID NO: 121
CDR-H2: IGTGGIT;
SEQ ID NO: 122
CDR-H3: VRSSGSDWWFHI;
SEQ ID NO: 123
CDR-L1: QSVYNNNN;
SEQ ID NO: 124
CDR-L2: RAS;
and
SEQ ID NO: 125
CDR-L3: QGYFSGFITT.

Single chain antibodies comprising CDRs SEQ ID NO: 120-125, specifically bind W68 in the context of DAGWGNL (SEQ ID NO: 1) in misfolded TDP-43. The single chain antibody comprises CDR sequences of antibody 28H3-28K1.

In an embodiment, the single chain antibody comprises a heavy chain variable region comprising: i) an amino acid sequence as set forth in SEQ ID NO: 128, ii) an amino acid sequence with at least 80%, at least 85%, at least 90% or at least 95% sequence identity to SEQ ID NO: 128, wherein the CDR sequences are as set forth in SEQ ID NOs: 120-122, or iii) a conservatively substituted amino acid sequence of i), and/or wherein the single chain antibody comprises a light chain variable region comprising an amino acid sequence as set forth in SEQ ID NO: 129, ii) an amino acid sequence with at least 80%, at least 85%, at least 90% or at least 95% sequence identity to SEQ ID NO: 129, wherein the CDR sequences are as set forth in SEQ ID NOs: 123-125, or iii) a conservatively substituted amino acid sequence of i), optionally wherein the heavy chain variable region amino acid sequence is encoded by a nucleotide sequence as set out in SEQ ID NO: 126 or a codon degenerate or optimized version thereof and/or the light chain variable region amino acid sequence is encoded by a nucleotide sequence as set out in SEQ ID NO: 127 or a codon degenerate or optimized version thereof.

In one embodiment, the single chain antibody has CDR sequences comprising

SEQ ID NO: 16
CDR-H1: GFSFSSNYV;
SEQ ID NO: 17
CDR-H2: IWFAGIVDTT;
SEQ ID NO: 18
CDR-H3: ARNPVGSVNL;
SEQ ID NO: 19
CDR-L1: ESVYSNNR;
SEQ ID NO: 20
CDR-L2: YAS;
and
SEQ ID NO: 21
CDR-L3: AGWRGARTDGVD.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 14H1-14K2.

In an embodiment, the single chain antibody comprises a heavy chain variable region comprising: i) an amino acid sequence as set forth in SEQ ID NO: 100, ii) an amino acid sequence with at least 80%, at least 85%, at least 90% or at least 95% sequence identity to SEQ ID NO: 100, wherein the CDR sequences are as set forth in SEQ ID NOs: 16-18, or iii) a conservatively substituted amino acid sequence of i), and/or wherein the single chain antibody comprises a light chain variable region comprising an amino acid sequence as set forth in SEQ ID NO: 101, ii) an amino acid sequence with at least 80%, at least 85%, at least 90% or at least 95% sequence identity to SEQ ID NO: 101, wherein the CDR sequences are as set forth in SEQ ID NOs: 19-21, or iii) a conservatively substituted amino acid sequence of i), optionally wherein the heavy chain variable region amino acid sequence is encoded by a nucleotide sequence as set out in SEQ ID NO: 78 or a codon degenerate or optimized version thereof and/or the light chain variable region amino acid sequence is encoded by a nucleotide sequence as set out in SEQ ID NO: 79 or a codon degenerate or optimized version thereof.

In one embodiment, the single chain antibody has CDR sequences comprising:

SEQ ID NO: 22
CDR-H1: GFSFSSSYV;
SEQ ID NO: 23
CDR-H2: SDTGINT;
SEQ ID NO: 24
CDR-H3: ARRYTGDTYLGNFNL;
SEQ ID NO: 25
CDR-L1: QSVYKNNY;
SEQ ID NO: 26
CDR-L2: KAS;
and
SEQ ID NO: 27
CDR-L3: AGGWRSLNA.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 17.

In an embodiment, the single chain antibody comprises a heavy chain variable region comprising: i) an amino acid sequence as set forth in SEQ ID NO: 102, ii) an amino acid sequence with at least 80%, at least 85%, at least 90% or at least 95% sequence identity to SEQ ID NO: 102, wherein the CDR sequences are as set forth in SEQ ID NOs: 22-24, or iii) a conservatively substituted amino acid sequence of i), and/or wherein the antibody comprises a light chain variable region comprising an amino acid sequence as set forth in SEQ ID NO: 103, ii) an amino acid sequence with at least 80%, at least 85%, at least 90% or at least 95% sequence identity to SEQ ID NO: 103, wherein the CDR sequences are as set forth in SEQ ID NOs: 25-27, or iii) a conservatively substituted amino acid sequence of i), optionally wherein the heavy chain variable region amino acid sequence is encoded by a nucleotide sequence as set out in SEQ ID NO: 80 or a codon degenerate or optimized version thereof and/or the light chain variable region amino acid sequence is encoded by a nucleotide sequence as set out in SEQ ID NO: 81 or a codon degenerate or optimized version thereof.

In one embodiment, the single chain antibody has CDR sequences comprising:

SEQ ID NO: 28
CDR-H1: EFSFSSRYW;
SEQ ID NO: 29
CDR-H2: IYTGSIDAT;
SEQ ID NO: 30
CDR-H3: VRGSDAWGLYFNL;
SEQ ID NO: 31
CDR-L1: QSIHKNNY;
SEQ ID NO: 32
CDR-L2: FAS;
and
SEQ ID NO: 33
CDR-L3: AGVYSGRIFA.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 20.

In an embodiment, the single chain antibody comprises a heavy chain variable region comprising: i) an amino acid sequence as set forth in SEQ ID NO: 104, ii) an amino acid sequence with at least 80%, at least 85%, at least 90% or at least 95% sequence identity to SEQ ID NO: 104, wherein the CDR sequences are as set forth in SEQ ID NOs: 28-30, or iii) a conservatively substituted amino acid sequence of i), and/or wherein the antibody comprises a light chain variable region comprising an amino acid sequence as set forth in SEQ ID NO: 105, ii) an amino acid sequence with at least 80%, at least 85%, at least 90% or at least 95% sequence identity to SEQ ID NO: 105, wherein the CDR sequences are as set forth in SEQ ID NOs: 31-33, or iii) a conservatively substituted amino acid sequence of i), optionally wherein the heavy chain variable region amino acid sequence is encoded by a nucleotide sequence as set out in SEQ ID NO: 82 or a codon degenerate or optimized version thereof and/or the light chain variable region amino acid sequence is encoded by a nucleotide sequence as set out in SEQ ID NO: 83 or a codon degenerate or optimized version thereof.

In one embodiment, the single chain antibody has CDR sequences comprising:

SEQ ID NO: 34
CDR-H1: GFSLSSYT;
SEQ ID NO: 35
CDR-H2: IYGGIGST;
SEQ ID NO: 36
CDR-H3: GRGDI;
SEQ ID NO: 37
CDR-L1: QSVYKNR;
SEQ ID NO: 38
CDR-L2: GAS;
and
SEQ ID NO: 39
CDR-L3: LGNYDCSSVDCGA.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 30.

In an embodiment, the single chain antibody comprises a heavy chain variable region comprising: i) an amino acid sequence as set forth in SEQ ID NO: 106, ii) an amino acid sequence with at least 80%, at least 85%, at least 90% or at least 95% sequence identity to SEQ ID NO: 106, wherein the CDR sequences are as set forth in SEQ ID NOs: 34-36, or iii) a conservatively substituted amino acid sequence of i), and/or wherein the antibody comprises a light chain variable region comprising an amino acid sequence as set forth in SEQ ID NO: 107, ii) an amino acid sequence with at least 80%, at least 85%, at least 90% or at least 95% sequence identity to SEQ ID NO: 107, wherein the CDR sequences are as set forth in SEQ ID NOs: 37-39, or iii) a conservatively substituted amino acid sequence of i), optionally wherein the heavy chain variable region amino acid sequence is encoded by a nucleotide sequence as set out in SEQ ID NO: 84 or a codon degenerate or optimized version thereof and/or the light chain variable region amino acid sequence is encoded by a nucleotide sequence as set out in SEQ ID NO: 85 or a codon degenerate or optimized version thereof.

In one embodiment, the single chain antibody has CDR sequences comprising:

SEQ ID NO: 40
CDR-H1: GFSFSAYY;
SEQ ID NO: 41
CDR-H2: TIPIGRT;
SEQ ID NO: 42
CDR-H3: AGGPTGNSHFTL;
SEQ ID NO: 43
CDR-L1: ESVYNNNQ;
SEQ ID NO: 44
CDR-L2: QAS;
and
SEQ ID NO: 45
CDR-L3: AGYKSPTTDGIA.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 38.

In an embodiment, the single chain antibody comprises a heavy chain variable region comprising: i) an amino acid sequence as set forth in SEQ ID NO: 108, ii) an amino acid sequence with at least 80%, at least 85%, at least 90% or at least 95% sequence identity to SEQ ID NO: 108, wherein the CDR sequences are as set forth in SEQ ID NOs: 40-42, or iii) a conservatively substituted amino acid sequence of i), and/or wherein the antibody comprises a light chain variable region comprising an amino acid sequence as set forth in SEQ ID NO: 109, ii) an amino acid sequence with at least 80%, at least 85%, at least 90% or at least 95% sequence identity to SEQ ID NO: 109, wherein the CDR sequences are as set forth in SEQ ID NOs: 43-45, or iii) a conservatively substituted amino acid sequence of i), optionally wherein the heavy chain variable region amino acid sequence is encoded by a nucleotide sequence as set out in SEQ ID NO: 86 or a codon degenerate or optimized version thereof and/or the light chain variable region amino acid sequence is encoded by a nucleotide sequence as set out in SEQ ID NO: 87 or a codon degenerate or optimized version thereof.

In one embodiment, the single chain antibody has CDR sequences comprising:

SEQ ID NO: 46
CDR-H1: GFSLSSYA;
SEQ ID NO: 47
CDR-H2: IYNYET;
SEQ ID NO: 48
CDR-H3: ARDIFRTNTNL;
SEQ ID NO: 49
CDR-L1: QSVYKNNG;
SEQ ID NO: 50
CDR-L2: FTS;
and
SEQ ID NO: 51
CDR-L3: LGGYDCSSRVCGA.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 36.

In an embodiment, the single chain antibody comprises a heavy chain variable region comprising: i) an amino acid sequence as set forth in SEQ ID NO: 110, ii) an amino acid sequence with at least 80%, at least 85%, at least 90% or at least 95% sequence identity to SEQ ID NO: 110, wherein the CDR sequences are as set forth in SEQ ID NOs: 46-48, or iii) a con-servatively substituted amino acid sequence of i), and/or wherein the antibody comprises a light chain variable region comprising an amino acid sequence as set forth in SEQ ID NO: 111, ii) an amino acid sequence with at least 80%, at least 85%, at least 90% or at least 95% sequence identity to SEQ ID NO: 111, wherein the CDR sequences are as set forth in SEQ ID NOs: 49-51, or iii) a conservatively substituted amino acid sequence of i), optionally wherein the heavy chain variable region amino acid sequence is encoded by a nucleotide sequence as set out in SEQ ID NO: 88 or a codon degenerate or optimized version thereof and/or the light chain variable region amino acid sequence is encoded by a nucleotide sequence as set out in SEQ ID NO: 89 or a codon degenerate or optimized version thereof.

In one embodiment, the single chain antibody has CDR sequences comprising:

SEQ ID NO: 140
CDR-H1: GFTFSSYY;
SEQ ID NO: 141
CDR-H2: INTNGGST;
SEQ ID NO: 142
CDR-H3: VRQNYEGAY;
SEQ ID NO: 143
CDR-L1: QSIVHSNGNTY;
SEQ ID NO: 144
CDR-L2: KVS;
and
SEQ ID NO: 145
CDR-L3: FQSSHVPWT.

Single chain antibodies comprising CDRs SEQ ID NO: 16-21, specifically bind W68 in the context of DAGWGNL (SEQ ID NO: 1) in misfolded TDP-43. In one embodiment, the single chain antibody comprises CDR sequences of antibody 3F11.

In an embodiment, the antibody comprises a heavy chain variable region comprising: i) an amino acid sequence as set forth in SEQ ID NO: 148, ii) an amino acid sequence with at least 80%, at least 85%, at least 90% or at least 95% sequence identity to SEQ ID NO: 148, wherein the CDR sequences are as set forth in SEQ ID NOs: 140-142, or iii) a conservatively substituted amino acid sequence of i), and/or wherein the antibody comprises a light chain variable region comprising an amino acid sequence as set forth in SEQ ID NO: 149, ii) an amino acid sequence with at least 80%, at least 85%, at least 90% or at least 95% sequence identity to SEQ ID NO: 149, wherein the CDR sequences are as set forth in SEQ ID NOs: 143-145, or iii) a conservatively substituted amino acid sequence of i), optionally wherein the heavy chain variable region amino acid sequence is encoded by a nucleotide sequence as set out in SEQ ID NO: 146 or a codon degenerate or optimized version thereof and/or the light chain variable region amino acid sequence is encoded by a nucleotide sequence as set out in SEQ ID NO: 147 or a codon degenerate or optimized version thereof.

The amino acid sequences of ii) can also have greater that 95% sequence identity. In various embodiments, the amino acid sequence has at least 96% identity to a sequence provided herein. In various embodiments, the amino acid sequence has at least 97% identity to a sequence provided herein. In various embodiments, the amino acid sequence has at least 98% identity to a sequence provided herein. In various embodiments, the amino acid sequence has at least 99% identity to a sequence provided herein.

In an embodiment, the single chain antibody comprises a heavy chain variable region comprising a conservatively substituted amino acid sequence as set forth in any in one of SEQ ID NOs: 98, 100, 102, 104, 106, 108, 110, 128, 138, or 148. In an embodiment, the single chain antibody comprises a heavy chain variable region comprising a conservatively substituted amino acid sequence as set forth in any in one of SEQ ID NOs: 99, 101, 103, 105, 107, 109, 111, 129, 139, or 149. For example, the heavy chain variable region and/or the light chain variable region, optionally framework region 1, 2 and/or 3, can include 1, 2, 3, 4 or 5 conservative amino acid substitutions.

In one embodiment, the single chain antibodies comprise a signal peptide. For example the signal peptide can be a heavy chain signal peptide or a light chain signal peptide. Exemplary heavy chain signal sequences include/comprise MNFGLRLILLVLVLKGVLC (native signal sequence for 2F7) (SEQ ID NO: 160), METGLRWLLLVAVLKGVQCQ (SEQ ID NO: 161), MELGLSWIFLLAILKGVQC (SEQ ID NO: 162), MELGLRVWVFLVAILEGVQC (SEQ ID NO: 163), MKHLWFFLLLVAAPRWVLS (SEQ ID NO: 164), MDWTWRILFLVAAATGAHS (SEQ ID NO: 165), MDWTWRFLFVVAAATGVQS (SEQ ID NO: 166), MEFGLSWLFLVAILKGVQC (SEQ ID NO: 167), MEFGLSWVFLVALFRGVQC (SEQ ID NO: 168) and MDLLHKNMKHLWFFLLLVAAPRVWVLS (SEQ ID NO: 169). Exemplary light chain signal sequences include MKLPVRLLVLMFWIPASSS (native signal sequence for 2F7) (SEQ ID NO: 170), MDMRVPAQLLGLLLLWLSGARC (SEQ ID NO: 171) and MKYLLPTAAAGLLLLAAQPAMA (SEQ ID NO: 172). Vector constructs comprising the secretable single chain antibodies can be used to provide a local extracellular depot of antibody for targeting secreted TDP-43 and minimizing cell to cell spread.

As shown herein, intrabodies described herein specifically bind and/or selectively bind misfolded TDP-43 and not native TDP-43. Selective binding can be measured using an ELISA or surface plasmon resonance measurement, as described herein. Intrabodies are antibody therapies intended to work inside a cell, blocking for example toxic proteins and preventing their spread to healthy cells.

The intrabodies are derived from mouse and rabbit monoclonal antibodies as described in the Examples.

The intrabodies can be humanized. The humanization of antibodies from non-human species (for example from mouse or rabbit) has been well described in the literature. See for example EP-B1 0 239400 and Carter & Merchant 1997 (Curr Opin Biotechnol 8, 449-454, 1997 incorporated by reference in their entirety herein). Humanized antibodies are also readily obtained commercially (e.g. Scotgen Limited, 2 Holly Road, Twickenham, Middlesex, Great Britain).

Humanized forms of rodent antibodies are readily generated by CDR grafting (Riechmann et al. Nature, 332:323-327, 1988). In this approach the six CDR loops comprising the antigen binding site of the rodent monoclonal antibody are linked to corresponding human framework regions. CDR grafting often yields antibodies with reduced affinity as the amino acids of the framework regions may influence antigen recognition (Foote & Winter. J Mol Biol, 224: 487-499, 1992). To maintain the affinity of the antibody, it is often necessary to replace certain framework residues by site directed mutagenesis or other recombinant techniques and may be aided by computer modeling of the antigen binding site (Co et al. J Immunol, 152: 2968-2976, 1994).

Humanized forms of antibodies are optionally obtained by resurfacing (Pedersen et al. J Mol Biol, 235: 959-973, 1994). In this approach only the surface residues of a rodent antibody are humanized.

In an embodiment, the single chain antibody is a chimeric antibody such as a humanized antibody

The linker linking the heavy and light variable chains should be at least 5 amino acids or larger, preferably at least or about 10 amino acids or at least or about 15 amino acids in length and less than for example about 25 amino acids. Linker sequences that can be used include (Gly-Gly-Gly-Gly-Ser)₃ (SEQ ID NO: 180), (Gly-Gly-Gly-Gly-Ser)₄ (SEQ ID NO: 181) and GSTGGGGSGKPGSGEGGGGS (SEQ ID NO: 182). Other linkers include Glu Ser Gly Arg Ser Gly Gly Gly Gly Ser Gly Gly Gly Gly Ser (SEQ ID NO: 183); Glu Gly Lys Ser Ser Gly Ser Gly Ser Glu Ser Lys Ser Thr (SEQ ID NO: 184), Glu Gly Lys Ser Ser Gly Ser Gly Ser Glu Ser Lys-Ser Thr Gln (SEQ ID NO: 185), Glu Gly Lys Ser Ser Gly Ser Gly Ser Glu Ser Lys Val Asp (SEQ ID NO: 186), Gly Ser Thr Ser Gly Ser Gly Lys Ser Ser Glu Gly Lys Gly (SEQ ID NO: 187), Lys Glu Ser Gly Ser Val Ser Ser Glu Gln Leu Ala Gln Phe Arg Ser Leu Asp (SEQ ID NO: 188), and Glu Ser Gly Ser Val Ser Ser Glu Glu Leu Ala Phe Arg Ser Leu Asp (SEQ ID NO:189). Where the single chain antibody comprises a detectable label such as a fusion tag or targeting moiety, it may be fused directly or indirectly, for example by way of a tag linker, or targeting moiety linker optionally wherein said linker comprises or is GGGGS (SEQ ID NO: 190). A further aspect is a single chain antibody conjugated to a detectable label. In an embodiment, the detectable label is a positron-emitting radionuclide. A positron-emitting radionuclide can be used for example in PET imaging. In such embodiments, the single chain antibody may comprise a cell penetrating moiety such as a fused cell penetrating peptide. The cell penetrating peptide may be derived from TAT, for example YGRKKRRQRRR (SEQ ID NO: 191), CYGRKKRRQRRRC (c-Tat) (SEQ ID NO: 192) or GDIMGEWGNEIFGAIAGFLGYGRKKRRQRRR (HA-Tat) (SEQ ID NO: 193), or other such as RRRRRRRR (R8) (SEQ ID NO: 194) and CRRRRRRRRC (cR8) (SEQ ID NO: 195), RQIKIWFQNRRMKWKK (penetratin) (SEQ ID NO: 196) and GWTLNSAGYLLGKINLKALAALAKKIL (transportan) (SEQ ID NO: 197). The C residues at each side of the CPP sequence permit formation of a disulfide bond and cyclisation. HA refers to the influenza virus haemagluttinin protein. The cell penetrating peptide can for example comprise a radionuclide suitable for PET imaging such as carbon-11, nitrogen-13, oxygen-15, fluorine-18, gallium-68, zirconium-89, rubidium-82 and yttrium 90. Methods for covalently linking the tracer molecule to a peptide are known in the art.

Accordingly, an embodiment provides an immunoconjugate comprising a single chain antibody described herein and a detectable label, such as a fusion tag or a targeting moiety.

The fusion tag, can be a FLAG tag or a MYC tag, and the tag can be N-terminal or C-terminal and conjugated directly or indirectly via a linker. In another embodiment, the single chain antibody is conjugated to a targeting moiety such as a lysosomal targeting signal.

In an embodiment, the targeting moiety is a lysosomal or autophagy targeting signal. For example, the lysosomal targeting moiety can be or comprise the sequence YPTL (SEQ ID NO: 173), KSIRSGYEVM (SEQ ID NO: 174), RWRKSHSSSYTPLSGSTYPEGRH (SEQ ID NO: 175). Other lysosomal targeting sequences that can be used include classical tyrosine-based NPXY (SEQ ID NO: 176) and YXXφ (SEQ ID NO: 177) sequences where X can be any amino acid residue and φ is a bulky hydrophobic residue, di-leucine-based [D/E]XXXL[L/I] (SEQ ID NO: 178) or DXXLL (SEQ ID NO: 179) sequences.

In one embodiment, the single chain antibody can comprising the antibody and linker portions and optionally lysosmal targeting moieties of any of SEQ ID NOs: 203, 205, 207, 209, 213, 215, 217, 219, 223, 225, 227, 229, 233, 235, 237, 239, 243, 245, 247, 249, 253, 255, 257 and/or 259 (e.g. excluding tags and sequence linking tags (tag linker) and including if not present a start methione).

The antibodies can be prepared as described in the Examples, for example the heavy chain and light chain variable regions can be cloned into an expression vector and expressed. If the antibodies are to be produced (e.g. purified), a signal sequence is utilized to direct secretion.

As described herein, the single chain antibodies are suitable as intrabodies for targeting intracellular misfolded aggregates of TDP-43. In such constructs, a signal sequence is not incorporated as the antibody is suitably expressed in the cell.

Accordingly, a further aspect is an isolated nucleic acid encoding a single chain antibody or immunoconjugate described herein.

Nucleic acids encoding the single chain antibodies and immunoconjugates described herein comprising a fused heavy chain and light chain are also provided, for example encoding a heavy chain comprising CDR-H1, CDR-H2 and CDR-H3 regions described herein and encoding a light chain comprising CDR-L1, CDR-L2 and CDR-L3 regions described herein and including the nucleic acid sequences described in any of Tables 3, 9, 10, 11, 12, 13 and 14 and those that encode any of the amino acid sequences described herein.

For example, the nucleic acid sequence comprises any one of SEQ ID NOs: 76-89, SEQ ID NOs: 126-127, SEQ ID NOs: 136-137, 146-147 and/or antibody and linker portions and optionally lysosmal targeting moieties of any of SEQ ID NOs: 202, 204, 206, 208, 212, 214, 216, 218, 222, 224, 226, 228, 232, 234, 236, 238, 242, 244, 246, 248, 252, 254, 256 and/or 258 (e.g. excluding tags and sequence linking tags (i.e. tag linker) and including if not present a start methionine codon and stop codon).

The present disclosure also provides variants of the nucleic acid sequences that encode the single chain antibodies disclosed herein.

For example, the variants include nucleotide sequences that hybridize to the nucleic acid sequences encoding the antibody disclosed herein under at least moderately stringent hybridization conditions or codon degenerate or optimized sequences. In another embodiment, the variant nucleic acid sequences have at least 70%, most preferably at least 80%, even more preferably at least 90% and even most preferably at least 95% sequence identity to nucleic acid sequences comprising any one of SEQ ID NOs: 76-89, SEQ ID NOs: 126-127, SEQ ID NOs: 136-137, SEQ ID Nos: 146-147 and/or antibody and linker portions and optionally lysosomal targeting moieties of any of SEQ ID NOs: 202, 204, 206, 208, 212, 214, 216, 218, 222, 224, 226, 228, 232, 234, 236, 238, 242, 244, 246, 248, 252, 254, 256 and/or 258. The variant sequences encode for example antibodies with conservative changes outside the CDR sequences and codon optimized versions of the antibody sequences described herein, for example optimized for expression in human cells.

Another aspect is an expression cassette or vector comprising the nucleic acid herein disclosed. The expression cassette can comprise for example the nucleic acid encoding the single chain antibody and linker, and regulatory sequences such as a promoter that is operatively linked to the nucleic acid. In an embodiment, the vector is an isolated vector.

The vector can be any vector, suitably an expression vector suitable for producing a single chain antibody described herein. In an embodiment, the vector is suitable for expressing for example single chain antibodies (e.g. intrabodies).

The nucleic acid molecules may be incorporated in a known manner into an appropriate expression vector which ensures expression of the protein, for example as described in the Examples.

Molecular cloning techniques known in the art can be used in vector construction. In an embodiment, an expression vector and the insert are digested with the appropriate restriction enzymes and ligated by T4 polymerase. The ligation reaction is transformed into competent cells. Individual colonies are picked, overnight cultures are grown, and DNA are purified for diagnostic restriction digest.

Possible expression vectors include but are not limited to cosmids, plasmids, or modified viruses (e.g. replication defective retroviruses, including lentiviral vectors, adenoviruses and adeno-associated viruses).

In one embodiment, the vector is an adeno associated virus capable of transducing neuronal cells (e.g. AAV serotype 9).

The vectors may comprise suitable regulatory sequences.

Suitable regulatory sequences may be derived from a variety of sources, including bacterial, fungal, viral, mammalian, or insect genes. Examples of such regulatory sequences include: a transcriptional promoter and enhancer or RNA polymerase binding sequence, a ribosomal binding sequence, including a translation initiation signal. Additionally, depending on the cell to be transfected/infected/transduced and the vector employed, other sequences, such as an origin of replication, additional DNA restriction sites, enhancers, and sequences conferring inducibility of transcription may be incorporated into the expression vector. In an embodiment, the regulatory sequences direct or increase expression in neural tissue and/or cells. In an embodiment, the vector is a viral vector. The recombinant expression vectors may also contain a marker gene which facilitates the selection of host cells transformed, infected or transfected with a vector for expressing an antibody described herein. The recombinant expression vectors may also contain other expression cassettes which encode for example a fusion moiety (e.g. for creating an antibody “fusion protein”) which can aid in the detection, including for example tags and labels described herein.

The nucleic acids and vectors can be used to deliver the single chain antibody to a cell for example cells in a subject.

A wide range of approaches to transduce the cells can be used, including viral vectors, “naked” DNA, DNA in lipid or other nanoparticles, adjuvant assisted DNA, gene gun, etc. For example, retroviral vectors such as lentiviral vectors can also be used to transduce cells with intrabodies. Other vector systems useful in practicing the present invention include the adenoviral and adeno associated virus based vectors.

Also provided in another aspect is a cell recombinantly expressing (e.g. a recombinant cell) a single chain antibody described herein and/or comprising a nucleic acid, expression cassette or vector encoding a single chain antibody described herein. In an embodiment, the cell is an isolated cell, expressing an antibody described herein or comprising a nucleic acid, expression cassette or vector herein disclosed.

In an embodiment, the cell is a cell capable of producing an antibody described herein.

### IV. Compositions

A further aspect is a composition comprising an isolated antibody, immunoconjugate, nucleic acid, expression cassette or vector described herein. Also provided is a composition comprising two or more antibodies, immunoconjugates, nucleic acids or vectors described herein.

In one embodiment, the composition comprises two or more isolated antibodies (e.g. two or more different antibodies).

In another embodiment, the composition comprises two or more immunoconjugates (e.g. two or more different immunoconjugates).

In another embodiment, the composition comprises two or more nucleic acids (e.g. two or more different nucleic acids).

In another embodiment, the composition comprises two or more expression cassettes (e.g. two or more different expression cassettes).

In another embodiment, the composition comprises two or more vectors (e.g. two or more different vectors).

In an embodiment, the two or more is two e.g. two antibodies or two immunoconjugates etc. In an embodiment, the two or more is three. In an embodiment, the two or more is four. In an embodiment, the two or more is five. In another embodiment, the two or more is six. In an embodiment, the two or more is seven.

Another aspect is a composition comprising a single chain antibody described herein. Also provided is a composition comprising two or more single chain antibodies described herein.

In one embodiment, the composition comprises a single chain antibody comprising the CDRs of antibody 2F7.

In another embodiment, the composition comprises a single chain antibody comprising the CDRs of antibody 1H3-1 K3.

In another embodiment, the composition comprises a single chain antibody comprising the CDRs of antibody 28H3-28K1.

In another embodiment, the composition comprises a single chain antibody comprising the CDRs of antibody 14H1-14K2.

In another embodiment, the composition comprises a single chain antibody comprising the CDRs of antibody 17.

In another embodiment, the composition comprises a single chain antibody comprising the CDRs of antibody 20.

In another embodiment, the composition comprises a single chain antibody comprising the CDRs of antibody 30.

In another embodiment, the composition comprises a single chain antibody comprising the CDRs of antibody 38.

In another embodiment, the composition comprises a single chain antibody comprising the CDRs of antibody 36.

In another embodiment, the composition comprises a single chain antibody comprising the CDRs of antibody 3F1.

The single chain antibody may comprise a lysosomal targeting moiety.

In one embodiment, the composition comprises a single chain antibody selected from the group consisting of LYS-2F7, LYS-28H3-28K1, LYS-14H1-14K2, YPTL-14H1-14K2, MYCH15L-2F7, MYCL15H-1H3-1K3, and MYCH20L-14H1-14K2.

In an embodiment, the composition comprises a single chain antibody selected from the group consisting of LYS-2F7 and MYCH15L-2F7.

In one embodiment, the composition comprises a single chain antibody selected from the group consisting of LYS-14H1-14K2, YPTL-14H1-14K2 and MYCH20L-14H1-14K2.

In another embodiment, the composition comprises two or more single chain antibodies selected from the group consisting of LYS-2F7, LYS-28H3-28K1, LYS-14H1-14K2, YPTL-14H1-14K2, MYCH15L-2F7, MYCL15H-1H3-1 K3, and MYCH20L-14H1-14K2.

In an embodiment, the two or more is two single chain antibodies. In an embodiment, the two or more is three. In an embodiment, the two or more is four. In an embodiment, the two or more is five. In an embodiment, the two or more is six. In an embodiment, the two or more is seven.

In an embodiment, the composition comprises a diluent. Suitable diluents for nucleic acids and vectors include but are not limited to water, saline solutions and ethanol.

The composition can comprise lipid particles such as liposomes, nanoparticles, or nanosomes for aiding delivering the nucleic acid and/or vectors.

Suitable diluents for polypeptides, including antibodies or fragments thereof and/or cells include but are not limited to saline solutions, pH buffered solutions and glycerol solutions or other solutions suitable for freezing polypeptides and/or cells.

In an embodiment, the composition comprises a nucleic acid or vector described herein. In another embodiment, the composition comprises an antibody or part thereof described herein and a diluent. In an embodiment, the composition is a sterile composition.

The composition can be formulated for intrathecal, intraparenchymal or intraventricular administration.

In an embodiment, the composition comprises a pharmaceutically acceptable carrier, diluent, and/or excipient. In an embodiment, the composition is for a method described herein such as targeting misfolded TDP-43.

In an embodiment, the composition is a pharmaceutical composition, for example for a method described herein such as for treating a subject in need thereof e.g. a subject with a TDP-43 proteinopathy.

The composition can comprise one or more antibodies described herein.

### VI. Methods

Included are methods for making the single chain antibodies, nucleic acids and vectors described herein.

In particular, provided are methods of making a single chain antibody, for example a single chain antibody selective for W68 in the context of DAGWGNL (SEQ ID NO:1). The CDRs can be grafted into a single chain antibody scaffold or the heavy chain and light chain variable regions can be amplified and cloned into a vector.

Vectors comprising single chain antibodies, optionally intrabodies, can be prepared by several methods. Molecular cloning techniques known in the art can be used in vector construction. As described in the Examples, the scFv nucleic acids were constructed in various formats, such as in a FLAG tag-VH-linker-VL-Lyslinker-lysosomal targeting sequence format. An antibody heavy chain variable domain (VH) and light chain variable domain (VL) can each consist of FR1-CDR1-FR2-CDR2-FR3-CDR3-FR4. Linkers for linking the VH and VL sequences and for linking the lysosomal targeting moiety can consist of 3 and 1 tandem repeats of GGGGS, respectively. The scFv nucleic acid, can be flanked by restriction sites such as 5′-NheI/HindIII-3′ restriction sequences, and be synthesized by sequentially linking individual phosphoramidite monomers using solid-phase phosphoramidite chemistry methods on a DNA synthesizer. The resulting single-stranded scFv gene insert can be amplified by standard PCR to generate double-stranded insert, which can then be cloned for example into a NheI/HindIII restriction enzyme digested vector such as pcDNA3.1(−) vector.

Other methods can also be used. For example mRNA can be isolated from a hybridoma cell line using a mRNA isolation kit (Qiagen, Chatsworth, Calif.). cDNA can be synthesized using for example Superscript First Strand, catalog no. 12371-019 (Invitrogen, Carlsbad, Calif.) with oligodT priming according to the manufacturers instructions. The variable regions of heavy chain (VH) and light chain (Vκ) can be amplified separately from first-strand cDNA by using a mixture of universal polymerase chain reaction (PCR) primers and Platinum Pfx DNA polymerase (Invitrogen). The PCR products for heavy chain and light chain can be cut with restriction enzymes such as PstI/BstEII and SacI/XhoI, respectively and agarose gel-purified. The cDNA inserts corresponding to VL and VH can be cloned into for example pBZUT7 vector and sequenced. The VH and VL domains can be assembled and linked together by PCR to yield the full-length scFv nucleic acid. The scFv nucleic acid can then be subcloned upstream of a Myc-tag. The construct can also be designed to include one or more moieties such as a signal sequence for efficient secretion, a tag such as c-myc epitope or FLAG to facilitate detection or a targeting moiety such as a lysosomal signal sequence or an autophagy signal sequence.

The structure of the resulting single chain antibody can be (HC FR1-HC CDR1-HC FR2-HC CDR2-HC FR3-HC CDR3-HC FR4)_{VH}-linker—(LC FR1-LC CDR1-LC FR2-LC CDR2-LC FR3-LC CDR3-LC FR4)_{VL}— optionally a Lys linker-optionally a targeting moiety such as a lysosomal targeting sequence, wherein HC FR1 is heavy chain framework 1 region, HC FR2 is heavy chain framework 2 region, HC FR3 is heavy chain framework 3 region, HC FR4 is heavy chain framework 4 region, wherein LC FR1 is light chain framework 1 region, LC FR2 is light chain framework 2 region, LC FR3 is light chain framework 3 region, LC FR4 is light chain framework 4 region.

Also provided are methods of detecting misfolded TDP-43, for example in a cell or in a subject.

A labelled single chain antibody (produced by adding a secretion signal) described herein can also be administered to a subject to detect the location of misfolded TDP-43. The measuring may for example by immunofluorescence or PET tracer. The methods may also include colocalization staining for example pan-TDP-43 staining.

Also provided is a method of decreasing intracellular misfolded TDP-43 levels, the method comprising administering a nucleic acid, vector or composition comprising said nucleic acid or vector described herein to a subject in need thereof, e.g a subject suspected of having, at risk of developing or diagnosed with a TDP-43 proteinopathy.

Also provided is a method of treating a subject, for example with a TDP-43 proteinopathy, the method comprising administering a nucleic acid, expression cassette, vector, single chain antibody, or composition comprising said nucleic acid, expression cassette or vector or single chain antibody described herein to a subject in need thereof.

In an embodiment, the TDP-43 proteinopathy is selected from amyotrophic lateral sclerosis (ALS), frontotemporal lobar degeneration (FTLD-TDP), primary lateral sclerosis, progressive muscular atrophy, and limbic-predominant age-related TDP-43 encephalopathy (LATE).

In some embodiments, the nucleic acid, vector or composition is administered to a subject in combination with another TDP-43 proteinopathy treatment. Other TDP-43 proteinopathy treatments include, but are not limited to treatments for ALS such as Riluzole (Rilutek or Tiglutik™), Edaravone (Radicava™), and Nuedexta™ (combination of dextromethorphan and quinidine).

The nucleic acid or vector can for example be comprised in a composition as described herein for example in combination with a pharmaceutically acceptable carrier, diluent and/or excipient and formulated for example in nanoparticles, or nanosomes for aiding delivering the nucleic acid and/or vectors.

The compositions, antibodies, immunoconjugates, nucleic acids and vectors described herein can be administered for example, by parenteral, intravenous, subcutaneous, intramuscular, intracranial, intraventricular, intrathecal, intraorbital, ophthalmic, intraspinal, intracisternal, intraperitoneal, intranasal, aerosol or oral administration.

Other embodiments contemplate the co-administration of the compositions, antibodies, immunoconjugates, nucleic acid and vectors described herein with biologically active molecules known to facilitate the transport across the blood brain barrier.

Also contemplated in certain embodiments, are methods for administering the compositions, antibodies, immunoconjugates, nucleic acids and vectors described herein across the blood brain barrier such as those directed at transiently increasing the permeability of the blood brain barrier as described in U.S. Pat. No. 7,012,061 “Method for increasing the permeability of the blood brain barrier”, herein incorporated by reference.

Also contemplated herein is the viral delivery of the compositions and/or nucleic acids described herein for expression of one or more antibodies described herein in a subject in need thereof or in a cell. An aspect includes a method of treating a subject with a TDP-43 proteinopathy comprising administering to a subject in need thereof an effective amount of a vectorized antibody of the disclosure described herein, or a composition comprising said vectorized antibody, optionally in combination with another TDP-43 proteinopathy treatment. In one embodiment, the vectorized antibody is a viral vector comprising a nucleic acid encoding an antibody described therein. In one embodiment, the method is for intracellular expression of an intrabody in a subject in need thereof. Intrabodies can for example inhibit intracellular misfolded TDP-43 aggregation or promote clearance of misfolded aggregates.

For example, Viral vectors such as adeno-associated virus (AAV, for example AAV9) and lentiviral vectors etc can used. Non-viral vectors can also be used. In certain embodiments, the nucleic acid, vector or composition can be injected intraventricularly or intrathecally. In other embodiments, the nucleic acid, vector or composition could be administered intravenously or subcutaneously or intramuscularly using for example a depot for sustained production of secreted single chain antibody.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Detection of Misfolded TDP-43

Antibodies were raised in rabbits using an immunogen comprising SEQ ID NO: 1. Antibodies were affinity purified. The affinity purified antibodies were tested for their ability to bind native TDP-43 polypeptide as well as misfolded TDP-43 polypeptide in a cell transfection assay using immunocytochemistry.

### Results:

HEK293 cells transfected by an HA-tagged TDP-43 construct with a triple missense tandem mutation in the nuclear localization signal, display TDP-43 aggregates in the cytoplasm, as detected by the HA antibody. HA-positive aggregates are also apparent for ΔNLS-TDP-43 W68S transfected cells. The affinity purified polyclonal antibodies recognize aggregates in the ΔNLS-TDP-43 transfected cells, comprising tryptophan 68 residue (Trp68), but do not show the same reactivity when Trp68 is mutated to serine. Anti-DAGWGNL (SEQ ID NO: 1) polyclonal rabbit antibodies therefore have selectivity for misfolded TDP-43 NTD comprising Trp68 residue.

When HA-tagged wild-type TDP-43 is overexpressed without an alteration to the nuclear localization signal, it generally localizes to the nucleus, and nuclear TDP-43 does not bind GS240 or GS243 affinity purified sera. Interestingly, GS240 and GS243 still bind wild-type TDP-43 when it is present in the cytoplasm of these cells, suggesting that the N-terminal ubiquitin like domain (NTD) can be misfolded in WT-TDP43 when mislocalized to the cytoplasm.

GS240 and GS243 polyclonal antibodies show minimal reactivity in the absence of TDP-43 aggregates. Reactivity of both antibodies is selective for misfolded TDP-43 in aggregates in the cytoplasm and requires the presence of Trp68.

### Example 2

### Mouse Monoclonal Antibody Production

Mouse monoclonal antibodies were produced using a peptide comprising DAGWGNL (SEQ ID NO: 1) e.g. DAGWGNLc (SEQ ID NO: 9), linked to KLH. Hybridomas were developed and analysed. Positive clones were identified and isotyped

### Example 3

### Rabbit Monoclonal Antibody Production

The DAGWGNL (SEQ ID NO: 1) peptide conjugated to KLH via a C-terminal cysteine (Peptide-KLH) was used for rabbit immunization for generation of B cells secreting monoclonal antibodies specific to the TDP-43 peptide. Based on indirect ELISA testing of the isolated B cells, B cells were selected for RNA isolation and generation of recombinant plasmid DNA. Functional, antigen-binding recombinant mAbs were be transfected and expressed for generation of purified mAb.

### Example 4

### Monoclonal Antibody Cloning and Sequencing

### Mouse mAb Cloning

Variable regions of the heavy and light chain immunoglobulin gene for several mouse hybridoma clones were identified and sequenced.

Sequences are shown in Tables 2, 3 and 4 below, with CDR1, CDR2 and CDR3 regions of heavy and light chains shown as underlined and bolded in Tables 3 and 4.

### Rabbit mAb Cloning

Antibody RNA isolation and generation of recombinant plasmid DNA was performed. Heavy and light chain variable regions were cloned into separate mammalian expression vectors, containing the rabbit heavy and kappa constant regions.

Top recombinant mAbs (DNA construct containing one heavy and one light chain) were sequenced, as shown in Tables 2, 3 and 4 below, with CDR1, CDR2 and CDR3 regions of heavy and light chains shown as underlined and bolded in Tables 3 and 4.

TABLE 2
Complementarity determining region (CDR) sequences

| | | | SEQ | | | SEQ |
|---|---|---|---|---|---|---|
| | | Amino acid | ID | | Amino acid | ID |
| Clone | CDR | sequence | NO: | CDR | sequence | NO: |
| 1H31K3 | H1 | GFSLSRYY | 10 | L1 | ESVYNNNH | 13 |
| | H2 | IIPGGTT | 11 | L2 | EAS | 14 |
| | H3 | AGGPTGNSHFTL | 12 | L3 | SGYKRVTTDGIA | 15 |
| 14H1K2 | H1 | GFSFSSNYV | 16 | L1 | ESVYSNNR | 19 |
| | H2 | IWFAGIVDTT | 17 | L2 | YAS | 20 |
| | H3 | ARNPVGSVNL | 18 | L3 | AGWRGARTDGVD | 21 |
| 17 | H1 | GFSFSSSYV | 22 | L1 | QSVYKNNY | 25 |
| | H2 | SDTGINT | 23 | L2 | KAS | 26 |
| | H3 | ARRYTGDTYLGNFNL | 24 | L3 | AGGWRSLNA | 27 |
| 20 | H1 | EFSFSSRYW | 28 | L1 | QSIHKNNY | 31 |
| | H2 | IYTGSIDAT | 29 | L2 | FAS | 32 |
| | H3 | VRGSDAWGLYFNL | 30 | L3 | AGVYSGRIFA | 33 |
| 30 | H1 | GFSLSSYT | 34 | L1 | QSVYKNR | 37 |
| | H2 | IYGGIGST | 35 | L2 | GAS | 38 |
| | H3 | GRGDI | 36 | L3 | LGNYDCSSVDCGA | 39 |
| 38 | H1 | GFSFSAYY | 40 | L1 | ESVYNNNQ | 43 |
| | H2 | TIPIGRT | 41 | L2 | QAS | 44 |
| | H3 | AGGPTGNSHFTL | 42 | L3 | AGYKSPTTDGIA | 45 |
| 36 | H1 | GFSLSSYA | 46 | L1 | QSVYKNNG | 49 |
| | H2 | IYNYET | 47 | L2 | FTS | 50 |
| | H3 | ARDIFRTNTNL | 48 | L3 | LGGYDCSSRVCGA | 51 |
| 28H3K1 | H1 | GFSLSSYN | 120 | L1 | QSVYNNNN | 123 |
| | H2 | IGTGGIT | 121 | L2 | RAS | 124 |
| | H3 | VRSSGSDWWFHI | 122 | L3 | QGYFSGFITT | 125 |
| 3E8 | H1 | GFTFSSYY | 130 | L1 | QSIVHSNGNTY | 133 |
| | H2 | INSNGGST | 131 | L2 | KVS | 134 |
| | H3 | VRQNYEGAY | 132 | L3 | FQSSHVPWT | 135 |
| 2F7 | H1 | GFTFSSYY | 130 | L1 | QSIVHSNGNTY | 133 |
| | H2 | INSNGGST | 131 | L2 | KVS | 134 |
| | H3 | VRQNYEGAY | 132 | L3 | FQSSHVPWT | 135 |
| 3F11 | H1 | GFTFSSYY | 140 | L1 | QSIVHSNGNTY | 143 |
| | H2 | INTNGGST | 141 | L2 | KVS | 144 |
| | H3 | VRQNYEGAY | 142 | L3 | FQSSHVPWT | 145 |

TABLE 3
DNA sequences of variable domain regions

| | | | SEQ ID |
|---|---|---|---|
| Clone | Isotype | DNA sequence | NO: |
| 1H3- | Heavy | CAGTCGGTGGAGGAGTCCGGGGGTCGCCTGGTCACGCCTGGGACACCCCTG | 76 |
| 1K3 | | ACACTCACCTGCACAGTCTCTGGATTCTCCCTCAGTAGGTACTACATGACCT | |
| | | GGGTCCGCCAGGCTCCAGGGAAGGGGCTGGAATACATCGGGGTCATTATTC | |
| | | CTGGTGGTACCACATACTACGCGAGCTGGGCGAAAGGCCGATTCACCATCTC | |
| | | CAAAACCTCGACCACGGTGGATCTGAGAATCACCAGTCCGACAACCGAGGAC | |
| | | ACGGCCACTTATTTCTGTGCCGGAGGTCCTACTGGTAACAGCCACTTTACAT | |
| | | TGTGGGGCCAGGGCACCCTGGTCACCGTCTC | |
| | Light | GTGATGACCCAGACTCCATCTTCCAAGTCTGTCCCTGTGGGAGGCACAGTCA | 77 |
| | | CCATCAATTGCCAGGCCAGTGAGAGTGTTTATAATAACAACCACTTATCCTG | |
| | | GTATCAGCAGAAATCAGGGCAGCCTCCCAAGCTCCTGATCTACGAAGCATCC | |
| | | AAACTGGAATCTGGGGTCCCACCGCGGTTCAAAGGCAGTGGATCTGGGACAC | |
| | | AGTTCACTCTCACCATCAGCGATGTGGTGTGTGACGATGCTGCCACTTACTAC | |
| | | TGTTCAGGATATAAACGTGTTACTACTGATGGTATTGCTTTCGGCGGAGGGA | |
| | | CCGAGGTGGTGGTCAAAG | |
| 14H1 | Heavy | CAGGAGCAGCTGGAGGAGTCCGGGGGAGACCTGGTCAAGCCTGAGGGATCC | 78 |
| K2 | | CTGACACTCACCTGCACAGCCTCTGGATTCTCCTTCAGTAGCAACTACGTGA | |
| | | TGTGCTGGGTCCGCCAGGCTCCAGGGAAGGGGCTGGAGTGGGTCGCATGCA | |
| | | TTTGGTTTGCTGGTATTGTTGATACTACTTACTACGCGACCTGGGCGAAAGGC | |
| | | CGATTCACCATCTCCAAAACCTCGTCGACCACGGTGACTCTGCAAATGACCA | |
| | | GTCTGACAGCCGCGGACACGGCCACCTATTTCTGTGCGAGAAATCCTGTTGG | |
| | | TAGTGTGAACTTGTGGGGCCAGGGCACCCTGGTCACCGTCTC | |
| | Light | GTGATGACCCAGACTCCATCTTCCAAGTCTGTCCCTGTGGGAGGCTCAGTCA | 79 |
| | | CCATCAATTGCCAGGCCAGTGAGAGTGTTTATAGTAACAACCGCTTATCCTG | |
| | | GTATCAGCAGAAACCAGGGCAGCCTCCTAAGCTCCTGATCTATTATGCATCCA | |
| | | CTCTGGAATCTGGGGTCCCATCGCGGTTCAAAGGCAGTGGATTTGGGACACA | |
| | | CTTCACTCTCACCATCAGCGGCGCGCAGTGTGACGATGCTGCCACTTACTAC | |
| | | TGTGCAGGATGGAGAGGTGCTAGGACTGATGGTGTAGATTTCGGCGGAGGG | |
| | | ACCGAGGTGGTGGTCAAAG | |
| 17 | Heavy | CAGGAGCAGCTGGTGGAGTCCGGGGGAGGCCTGGTCCAGCCTGGGGCATC | 80 |
| | | CCTGACACTCACCTGCACAGCCTCTGGATTCTCCTTCAGTAGCAGCTACGTG | |
| | | ATGTGCTGGGTCCGCCAGGCTCCAGGGAAGGGGCTGGAATGGATCACATGC | |
| | | AGTGATACTGGTATTAACACATGGTACGCGAGCTGGGCGAAAGGCCGATTC | |
| | | ACCATCTCCAAAACCTCGTCGACCACGGTGACTCTGCAAATGACCAGTCTGA | |
| | | CAGCCGCGGACACGGCCACCTATTTCTGTGCGAGACGTTATACTGGCGATA | |
| | | CTTATTTGGGAAACTTTAACTTGTGGGGCCAGGGCACCCTGGTCACCGTCTC | |
| | Light | GCCCAAGTGCTGACCCAGACTCCAGCCTCGGTGTCTGCAGCTGTGGGAGGC | 81 |
| | | ACAGTCACCATCAACTGCCAGGCCAGTCAGAGTGTTTATAAGAACAACTACT | |
| | | TATCCTGGTATCAGCAGAAACCAGGGCAGCCTCCCAAGCTCCTGATCTACAA | |
| | | GGCTTCCACTCTGGCATCTGGGGTCCCATCGCGGTTCAAGGGCAGTGGATCT | |
| | | GGGACACAGTTCACTCTCACCATCAGCGACGTGCAGTGTGACGATGCTGCCA | |
| | | CTTACTACTGTGCAGGCGGTTGGCGTAGTCTAAATGCTTTCGGCGGAGGGAC | |
| | | CGAGGTGGTGGTCAAAG | |
| 20 | Heavy | CAGGAGCAGCTGGAGGAGTCCGGGGGAGACCTGGTCAAGCCTGGGGCATC | 82 |
| | | CCTGACACTCACCTGCACAGCCTCTGAATTCTCCTTCAGTAGTAGATACTGG | |
| | | GCATGCTGGGTCCGCCAGGCTCCAGGGAAGGGGCTGGAGTGGAGCGCATG | |
| | | CATTTATACTGGTAGTATTGATGCTACTTACTACGCGAGCTGGGCGAAAGGC | |
| | | CGATTCACCATCTCCAAAACCTCGTCGACCACGGTGACTCTGCAAGTGACCA | |
| | | GTCTGACAGCCGCGGACACGGCCACCTATTTCTGTGTGAGGGGGAGTGATG | |
| | | CCTGGGGTCTCTACTTTAACTTGTGGGGCCAGGGCACCCTGGTCACCGTCTC | |
| | Light | TGCTGACCCAGACTCCATCCTCCGTGTCTGCAGCTGTGGGAGGCACAGTCAC | 83 |
| | | CGTCAGTTGCCAGTCCAGTCAGAGTATTCATAAGAATAATTACTTAGCCTGG | |
| | | TATCAGCAGAAACCAGGGCAGCCTCCCAAGCTCCTGATCTATTTTGCATCCAC | |
| | | TCTGGCATCTGGGGTCCCATCGCGGTTCAAAGGCAGTGGATCTGGGACACAG | |
| | | TTCACTCTCACCATCAGTGACCTGGAGTGTGACGATGCTGCCACTTACTACTG | |
| | | TGCAGGCGTTTATAGTGGTCGTATTTTTGCTTTCGGCGGAGGGACCGAGGTG | |
| | | GTGGTCAAAG | |
| 30 | Heavy | CAGTCGGTGGAGGAGTCCGGGGGTCGCCTGGTCACGCCTGGGACACCCCTG | 84 |
| | | ACACTCACCTGCAAAGTCTCTGGATTCTCCCTCAGTAGCTATACAATGATCTG | |
| | | GGTCCGCCAGGCTCCAGGGAAGGGGCTGGAGTGGATCGGGTACATTTATGG | |
| | | TGGTATTGGTAGCACATGGTACGCGAGCTGGGCGAAAGGCCGATTCACCAT | |
| | | CTCCAAAACCTCGACCACGGTGGATCTGAAAATCACCAGTCCGACAACCGAG | |
| | | GACACGGCCACCTATTTCTGTGGCAGAGGGGACATCTGGGGCCAGGGCACC | |
| | | CTGGTCACCGTCTC | |
| | Light | GTGCTGACCCAGACTGCATCCCCCGTGTCTGCGGCTGTGGGAGGCACAGTC | 85 |
| | | ACCATCAATTGCCAGTCCAGTCAGAGTGTTTATAAGAACCGCTTATCCTGGTA | |
| | | TCAGCAGAAACCAGGGCAGTCTCCCAAGCGCCTGATCTATGGTGCATCCACT | |
| | | CTGGAATCTGGGGTCCCATCGCGGTTCAAAGGCAGCGGATCTGGGACGCAG | |
| | | TTCACTCTCACCATCAGCGACGTGCAGTGTGACGATGCTGCCACTTACTACTG | |
| | | TCTAGGCAATTATGATTGTAGTAGTGTTGATTGTGGTGCTTTCGGCGGAGGG | |
| | | ACCGAGGTGGTGGTCAAAG | |
| 38 | Heavy | CAGTCGGTGGAGGAGTCCGGGGGTCGCCTGGTCACGCCTGGGACACCCCTG | 86 |
| | | ACACTCACCTGCACAGTCTCTGGATTCTCCTTCAGTGCCTACTACATGACCTG | |
| | | GGTCCGCCAGGCTCCAGGGAAGGGGCTGGAATTCATCGGAGTCACTATACC | |
| | | TATTGGCCGCACGTACTACGCGAGCTGGGCGAAAGGCCGATTCACCATCTCC | |
| | | AAAACCTCGACCACGGTGCATCTGAAAATCACCAGTCCGACAACCGAGGACA | |
| | | CGGCCGCTTATTTCTGTGCCGGAGGTCCTACTGGTAATAGCCACTTTACATT | |
| | | GTGGGGCCAGGGCACCCTGGTCACCGTCTC | |
| | Light | GTGATGACCCAGACTCCATCTTCCAAGTCTGTCCCTGTGGGAGACACAGTTA | 87 |
| | | CCATCAATTGCCAGGCCAGTGAGAGTGTTTATAATAACAACCAATTATCCTG | |
| | | GTATCAGCAGAAACCAGGGCAGCCTCCCAAGCTCCTGATCTACCAGGCATCC | |
| | | AAACTGGAATCTGGGGTCCCATCGCGGTTCAAAGGCAGTGGATCTGGGACAC | |
| | | AGTTCACTCTCACCATCAGCGATGTGGTGTGTGACGATGCTGCCACTTACTAC | |
| | | TGTGCAGGATATAAAAGTCCTACTACTGATGGTATTGCTTTCGGCGGAGGGA | |
| | | CCGAGGTGGTGGTCAAAG | |
| 36 | Heavy | CAGTCGGTGGAGGAGTCCGGGGGTCGCCTGGTCACGCCTGGGACACCCCTG | 88 |
| | | ACACTCACCTGCACAGTCTCTGGATTCTCCCTCAGTAGCTATGCAATGAGCT | |
| | | GGGTCCGCCAGGCTCCAGGGAAGGGGCTGGAATGGATCGGATTCATTTATA | |
| | | ATTATGAAACATACTACGCGAACTGGGCGAAAGGCCGATTCACCATCTCCAA | |
| | | AACCTCGACCTCGGTGGTTCTGAAAATCACCAGTCCGACAACCGACGACACG | |
| | | GCCACCTATTTCTGTGCCAGAGATATTTTTCGTACTAATACTAACTTGTGGGG | |
| | | CCAGGGCACCCTGGTCACCGTCTC | |
| | Light | GTGCTGACCCAGACTGCATCGCCCGTGTCTGCAGTTGTGGGAAGCACAGTCA | 89 |
| | | CCATCAATTGCCAGGCCAGTCAGAGTGTTTATAAGAACAACGGCTTATCCTG | |
| | | GTATCAGCAGAAACCAGGGCAGCCTCCCAAAGGCCTGATCTCTTTTACATCG | |
| | | ACTCTGGCATCTGGGGTCTCATCGCGGTTCAAAGGCAGTGGATCTGGGACAC | |
| | | AGTTTACTCTCACCATCAGCGACGTGCAGTGTGACGATGCTGCCACTTACTAC | |
| | | TGTCTAGGCGGTTATGATTGTAGTAGTCGTGTTTGTGGTGCTTTCGGCGGAG | |
| | | GGACCGAGGTGGTGGTCAAAG | |
| 28H3 | Heavy | CAGTCGCTGGAGGAGTCCGGGGGTCGCCTGGTCACGCCTGGGACACCCCTG | 126 |
| K1 | | ACACTCACCTGCACAGTCTCTGGATTCTCCCTCAGTAGCTACAACATGGGCT | |
| | | GGGTCCGCCAGGCTCCAGGGGAGGGGCTGGAGTGGATCGGAGTCATTGGT | |
| | | ACTGGTGGTATCACACACTACGCGACCTGGGCAAAAGGCCGAGTCGCCATC | |
| | | TCCAGAACCTCGACCACGGTGGGTCTGCGAATGACCAGTCCGACAACCGAG | |
| | | GACACGGCCACCTATTTCTGTGTCAGATCTAGTGGTAGTGATTGGTGGTTTC | |
| | | ACATCTGGGGCCAGGGCACCCTGGTCACCGTCTC | |
| | Light | GTGCTGACCCAGACTACATCGCCCGTGTCTGCAGCTGTGGGAGGCACAGTCA | 127 |
| | | CCATCAGTTGCCAGTCCAGTCAGAGTGTTTATAATAACAACAACTTAGCCTG | |
| | | GTTTCAGCAGAAACCAGGGCAGCCTCCCAAGCTCCTGATCTACAGGGCATCC | |
| | | AATCTGCCATCTGGTGTCCCATCGCGGTTCAGAGGCAGTGGATCTGGGTCAC | |
| | | AGTTCACTCTCACAATCAGCGAAGTACAGTGTGACGATGCTGCCACTTACTAC | |
| | | TGTCAAGGCTATTTTAGTGGATTTATCACTACTTTCGGCGGAGGGACCGAGG | |
| | | TGGTGGTCAAAG | |
| 3E8 | Heavy | GACGTGAAGCTCGTGGAGTCTGGGGGAGGCTTAGTGAAGCTTGGA | 136 |
| | | GGGTCCCTGAAACTCTCCTGTGCAGCCTCTGGATTCACTTTCAGTAGCTATT | |
| | | ACATGTCTTGGGTTCGCCAGACTCCAGAGAAGAGGCTGGAGTTGGTCGCA | |
| | | ACCATTAATAGTAATGGTGGTAGCACCTACTATCCAGACACTGTGAAGGGCC | |
| | | GAATCACCATCTCCAGAGACAATGCCAAGAACACCCTGCAGTTGCAAATGA | |
| | | GCAGTCTGAGGTCTGAGGACACAGCCTTGTATTACTGTGTAAGACAAAACT | |
| | | ACGAGGGGGCTTACTGGGGCCAAGGGACTCTGGTCACTGTCTCTGCA | |
| | Light | GATGTTTTGATGACCCAAACTCCACTCTCCCTGCCTGTCACTCTTGGAGATCA | 137 |
| | | AGCCTCCATCTCTTGCAGATCTAGTCAGAGCATTGTACATAGTAATGGAAAC | |
| | | ACCTATTTAGAATGGTACCTGCAGAAACCAGGCCAGTCTCCAAAGCTCCTGAT | |
| | | CTACAAAGTTTCCAACCGATTTTCTGGGGTCCCAGACAGGTTCAGTGGCAGT | |
| | | GGATCAGGGACAGATTTCACACTCAAGATCAGCAGAGTGGAGGCTGAGGATC | |
| | | TGGGAGTTTATTACTGCTTTCAAAGTTCACATGTTCCGTGGACGTTCGGTGGA | |
| | | GGCACCAAGCTGGAAATCAAA | |
| 2F7 | Heavy | GACGTGAAGCTCGTGGAGTCTGGGGGAGGCTTAGTGAAGCTTGGA | 136 |
| | | GGGTCCCTGAAACTCTCCTGTGCAGCCTCTGGATTCACTTTCAGTAGCTATT | |
| | | ACATGTCTTGGGTTCGCCAGACTCCAGAGAAGAGGCTGGAGTTGGTCGCA | |
| | | ACCATTAATAGTAATGGTGGTAGCACCTACTATCCAGACACTGTGAAGGGCC | |
| | | GAATCACCATCTCCAGAGACAATGCCAAGAACACCCTGCAGTTGCAAATGA | |
| | | GCAGTCTGAGGTCTGAGGACACAGCCTTGTATTACTGTGTAAGACAAAACT | |
| | | ACGAGGGGGCTTACTGGGGCCAAGGGACTCTGGTCACTGTCTCTGCA | |
| | Light | GATGTTTTGATGACCCAAACTCCACTCTCCCTGCCTGTCACTCTTGGAGATCA | 137 |
| | | AGCCTCCATCTCTTGCAGATCTAGTCAGAGCATTGTACATAGTAATGGAAAC | |
| | | ACCTATTTAGAATGGTACCTGCAGAAACCAGGCCAGTCTCCAAAGCTCCTGAT | |
| | | CTACAAAGTTTCCAACCGATTTTCTGGGGTCCCAGACAGGTTCAGTGGCAGT | |
| | | GGATCAGGGACAGATTTCACACTCAAGATCAGCAGAGTGGAGGCTGAGGATC | |
| | | TGGGAGTTTATTACTGCTTTCAAAGTTCACATGTTCCGTGGACGTTCGGTGGA | |
| | | GGCACCAAGCTGGAAATCAAA | |
| 3F11 | Heavy | GACGTGAAGCTCGTGGAGTCTGGGGGAGACTTAGTGAAGCTTGGAGGGTCC | 146 |
| | | CTGAAACTCTCCTGTGCAGCCTCTGGATTCACTTTCAGTAGCTATTACATGTC | |
| | | TTGGGTTCGCCAGACTCCAGAGAAGAGGCTGGAGTTGGTCGCAGTCATTAAT | |
| | | ACTAATGGTGGTAGCACCTACTATCCAGACACTGTGAAGGGCCGATTCACCA | |
| | | TCTCCAGAGACAATGCCAAGAACACCCTGTACCTGCAAATGAGCAGTCTGAA | |
| | | GTCTGAGGACACAGCCTTGTATTACTGTGTAAGACAAAACTACGAGGGGGCT | |
| | | TACTGGGGCCAAGGGACTCTGGTCACTGTCTCTGCA | |
| | Light | GATGTTTTGATGACCCAAACTCCACTCTCCCTGCCTGTCAGTCTTGGAGATCA | 147 |
| | | AGCCTCCATCTCTTGCAGATCTAGTCAGAGCATTGTACATAGTAATGGAAAC | |
| | | ACCTATTTAGAATGGTACCTGCAGAAACCAGGCCAGTCTCCAAAGCTCCTGAT | |
| | | CTACAAAGTTTCCAACCGATTTTCTGGGGTCCCAGACAGGTTCAGTGGCAGT | |
| | | GGATCAGGGACAGATTTCACACTCAAGATCAGCAGAGTGGAGGCTGAGGATC | |
| | | TGGGAGTTTATTACTGCTTTCAAAGTTCACATGTTCCGTGGACGTTCGGTGGA | |
| | | GGCACCAAGCTGGAAATCAAA | |

TABLE 4
Amino acid sequences of variable domain regions

| | | | SEQ |
|---|---|---|---|
| Clone | Isotype | Amino acid sequence | ID NO: |
| 1H3- | Heavy | QSVEESGGRLVTPGTPLTLTCTVSGFSLSRYYMTWVRQAPGKGLEYIGVIIPGGTTY | 98 |
| 1K3 | | YASWAKGRFTISKTSTTVDLRITSPTTEDTATYFCAGGPTGNSHFTLWGQGTLVTVS | |
| | Light | VMTQTPSSKSVPVGGTVTINCQASESVYNNNHLSWYQQKSGQPPKLLIYEASKLES | 99 |
| | | GVPPRFKGSGSGTQFTLTISDVVCDDAATYYCSGYKRVTTDGIAFGGGTEVVVK | |
| 14H1 | Heavy | QEQLEESGGDLVKPEGSLTLTCTASGFSFSSNYVMCWVRQAPGKGLEWVACIWFA | 100 |
| K2 | | GIVDTTYYATWAKGRFTISKTSSTTVTLQMTSLTAADTATYFCARNPVGSVNLWGQG | |
| | | TLVTVS | |
| | Light | VMTQTPSSKSVPVGGSVTINCQASESVYSNNRLSWYQQKPGQPPKLLIYYASTLES | 101 |
| | | GVPSRFKGSGFGTHFTLTISGAQCDDAATYYCAGWRGARTDGVDFGGGTEVVVK | |
| 17 | Heavy | QEQLVESGGGLVQPGASLTLTCTASGFSFSSSYVMCWVRQAPGKGLEWITCSDTGI | 102 |
| | | NTWYASWAKGRFTISKTSSTTVTLQMTSLTAADTATYFCARRYTGDTYLGNFNLWG | |
| | | QGTLVTVS | |
| | Light | AQVLTQTPASVSAAVGGTVTINCQASQSVYKNNYLSWYQQKPGQPPKLLIYKASTLA | 103 |
| | | SGVPSRFKGSGSGTQFTLTISDVQCDDAATYYCAGGWRSLNAFGGGTEVVVK | |
| 20 | Heavy | QEQLEESGGDLVKPGASLTLTCTASEFSFSSRYWACWVRQAPGKGLEWSACIYTGS | 104 |
| | | IDATYYASWAKGRFTISKTSSTTVTLQVTSLTAADTATYFCVRGSDAWGLYFNLWGQ | |
| | | GTLVTVS | |
| | Light | LTQTPSSVSAAVGGTVTVSCQSSQSIHKNNYLAWYQQKPGQPPKLLIYFASTLASGV | 105 |
| | | PSRFKGSGSGTQFTLTISDLECDDAATYYCAGVYSGRIFAFGGGTEVVVK | |
| 30 | Heavy | QSVEESGGRLVTPGTPLTLTCKVSGFSLSSYTMIWVRQAPGKGLEWIGYIYGGIGST | 106 |
| | | WYASWAKGRFTISKTSTTVDLKITSPTTEDTATYFCGRGDIWGQGTLVTVS | |
| | Light | VLTQTASPVSAAVGGTVTINCQSSQSVYKNRLSWYQQKPGQSPKRLIYGASTLESG | 107 |
| | | VPSRFKGSGSGTQFTLTISDVQCDDAATYYCLGNYDCSSVDCGAFGGGTEVVVK | |
| 38 | Heavy | QSVEESGGRLVTPGTPLTLTCTVSGFSFSAYYMTWVRQAPGKGLEFIGVTIPIGRTYY | 108 |
| | | ASWAKGRFTISKTSTTVHLKITSPTTEDTAAYFCAGGPTGNSHFTLWGQGTLVTVS | |
| | Light | VMTQTPSSKSVPVGDTVTINCQASESVYNNNQLSWYQQKPGQPPKLLIYQASKLES | 109 |
| | | GVPSRFKGSGSGTQFTLTISDVVCDDAATYYCAGYKSPTTDGIAFGGGTEVVVK | |
| 36 | Heavy | QSVEESGGRLVTPGTPLTLTCTVSGFSLSSYAMSWVRQAPGKGLEWIGFIYNYETYY | 110 |
| | | ANWAKGRFTISKTSTSVVLKITSPTTDDTATYFCARDIFRTNTNLWGQGTLVTVS | |
| | Light | VLTQTASPVSAVVGSTVTINCQASQSVYKNNGLSWYQQKPGQPPKGLISFTSTLASG | 111 |
| | | VSSRFKGSGSGTQFTLTISDVQCDDAATYYCLGGYDCSSRVCGAFGGGTEVVVK | |
| 28 | Heavy | QSLEESGGRLVTPGTPLTLTCTVSGFSLSSYNMGWVRQAPGEGLEWIGVIGTGGITH | 128 |
| | | YATWAKGRVAISRTSTTVGLRMTSPTTEDTATYFCVRSSGSDWWFHIWGQGTLVTV | |
| | | S | |
| | Light | VLTQTTSPVSAAVGGTVTISCQSSQSVYNNNNLAWFQQKPGQPPKLLIYRASNLPSG | 129 |
| | | VPSRFRGSGSGSQFTLTISEVQCDDAATYYCQGYFSGFITTFGGGTEVVVK | |
| 3E8 | Heavy | DVKLVESGGGLVKLGGSLKLSCAASGFTFSSYYMSVWRQTPEKRLELVATINSNGG | 138 |
| | | STYYPDTVKGRITISRDNAKNTLQLQMSSLRSEDTALYYCVRQNYEGAYWGQGTLVT | |
| | | VSA | |
| | Light | DVLMTQTPLSLPVTLGDQASISCRSSQSIVHSNGNTYLEWYLQKPGQSPKLLIYKVSN | 139 |
| | | RFSGVPDRFSGSGSGTDFTLKISRVEAEDLGVYYCFQSSHVPWTFGGGTKLEIK | |
| 2F7 | Heavy | DVKLVESGGGLVKLGGSLKLSCAASGFTFSSYYMSVWRQTPEKRLELVATINSNGG | 138 |
| | | STYYPDTVKGRITISRDNAKNTLQLQMSSLRSEDTALYYCVRQNYEGAYWGQGTLVT | |
| | | VSA | |
| | Light | DVLMTQTPLSLPVTLGDQASISCRSSQSIVHSNGNTYLEWYLQKPGQSPKLLIYKVSN | 139 |
| | | RFSGVPDRFSGSGSGTDFTLKISRVEAEDLGVYYCFQSSHVPWTFGGGTKLEIK | |
| 3F11 | Heavy | DVKLVESGGDLVKLGGSLKLSCAASGFTFSSYYMSVWRQTPEKRLELVAVINTNGGS | 148 |
| | | TYYPDTVKGRFTISRDNAKNTLYLQMSSLKSEDTALYYCVRQNYEGAYWGQGTLVT | |
| | | VSA | |
| | Light | DVLMTQTPLSLPVSLGDQASISCRSSQSIVHSNGNTYLEWYLQKPGQSPKLLIYKVSN | 149 |
| | | RFSGVPDRFSGSGSGTDFTLKISRVEAEDLGVYYCFQSSHVPWTFGGGTKLEIK | |

### Example 5

### Direct Binding Assays

Binding of antisera, hybridoma supernatants or purified antibodies to peptides (conjugated to BSA) can be examined by surface plasmon resonance using a Biacore™ 3000 instrument (GE Healthcare).

Binding analysis is carried out using a high density (at least 1000 response units (RU)) of antigen immobilized on flow cells. Dilutions of a selected clone are sequentially injected over the surface to assess binding.

For affinity kinetics and specificity analysis, a peptide comprising DAQWGNL (SEQ ID NO: 1) or GWG and conjugated to BSA, is immobilized at low densities (50-100 RU) on adjacent flow cells. Serial 2-fold dilutions of a selected clone (4.7 nM to 75 nM) are then sequentially injected over the surfaces at 60 μl/minute for 3 minutes, followed by a dissociation phase. Following a double-reference subtraction, the sensorgrams are fitted to a Langmuir 1:1 binding model. Up to three separate analyses are performed on 3 consecutive days using the same sensorchip and the same conditions.

Binding analysis can also be carried out also using Molecular Affinity Screening System (MASS-2) (Sierra Sensors GmbH, Hamburg, Germany). MASS-2 is a Surface Plasmon Resonance (SPR) Imaging analytical biosensor that employs high intensity laser light and high speed optical scanning to monitor binding interactions in real time. The peptide-BSA conjugates are covalently immobilized on separate flow cells of a High Amine Capacity (HAC) sensor chip, using standard amine-coupling chemistry, and unreacted sites blocked. Adjacent flow cells are similarly immobilized with BSA as a reference control surface.

### Binding Kinetics of Monoclonal Antibodies

Surface plasmon resonance (SPR) analysis was used to measure the binding kinetics of monoclonal antibodies to the peptide epitope. Peptide conjugated to bovine serum albumin (BSA) was immobilized at a very low density (approximately 50 RUs) on flow cells of a sensor chip. Purified mouse or rabbit monoclonal antibodies diluted 4-fold from 31.25 nM to 0.24 nM were injected sequentially over the surfaces for approximately 5 min followed by dissociation in buffer and surface regeneration. Binding parameters were calculated using kinetic curve fitting and a Langmuir 1:1 interaction model. Both mouse and rabbit monoclonal antibodies showed high subnanomolar affinity for the peptide epitope, as shown in Table 5 below. Rabbit monoclonal antibodies showed greater affinity (10⁻¹¹ nM range) compared to mouse monoclonal antibodies (10⁻¹⁰ nM range).

TABLE 5
Binding kinetics of monoclonal antibodies

| mAb | k_{ON} (M⁻¹s⁻¹) | k_{OFF} (s⁻¹) | K_{D}(M) |
|---|---|---|---|
| Mouse 2F7 | 2.83E+06 | 1.76E−03 | 6.22E−10 |
| Mouse 3E8 | 2.77E+06 | 2.00E−03 | 7.22E−10 |
| Rabbit | 4.03E+05 | 2.50E−05 | 6.20E−11 |
| 1H3-1K3 | | | |
| Rabbit | 4.70E+05 | 4.32E−05 | 9.19E−11 |
| 17H3-17K3 | | | |
| Rabbit | 5.99E+05 | 9.04E−06 | 1.51E−11 |
| 14H1-14K2 | | | |

### Example 6

### Epitope Recognized by Antibody in Denatured but not Natively Folded TDP N-Terminal Domain

The N-terminal domain of TDP-43 (residues 1-80) was expressed from plasmid in Escherichia coli and purified as described previously (Wang et al. 2018 EMBO). For Western blot analysis, native-PAGE was carried out using the Novex Bis-Tris system according to the manufacturer's specifications. Denaturing SDS-PAGE was carried out using the Novex Bis-Tris system according to the manufacturer's specifications. Blots were blocked in 5% milk powder in TBST, and then were incubated with purified rabbit polyclonal antibody GS240 overnight at 4° C. For detection on the ChemiDoc MP (Biorad, USA), a donkey anti-Rabbit IgG HRP-labelled secondary antibodies (GE Healthcare Life Sciences, USA) was used. The SuperSignal West Femto (Thermo Scientific, USA) substrate was used according to the manufacturer's instructions. The antibody only recognized and stained denatured TDP-43 NTD on the SDS-PAGE gel, and not TDP-43 NTD on the native PAGE gel thereby confirming that the epitope is not accessible in natively folded TDP-43 and only becomes exposed upon misfolding. Size exclusion chromatography (SEC) shows that TDP-43 NTD in native form or denatured form remains monomeric. Molecular weight markers are superimposed for reference.

### Example 7

### Immunohistochemistry of Patient Samples

Brain and spinal cord samples were obtained from The Netherlands Brain Bank and were processed and stained. Sections (8 μM thick) from formalin-fixed, paraffin-embedded tissue were deparaffinized and subsequently immersed in 0.3% H₂O₂ in phosphate-buffered saline (PBS) for 30 min to quench endogenous peroxidase activity. Formalin fixation forms protein cross-links that could mask antigenic sites in tissue specimens. To break protein cross-links, slides were pre-treated with heat and Tris-EDTA (pH 9). Primary test antibodies were incubated overnight at 4° C. at a dilution of 1:4000 (rabbit polyclonal GS240 antibody, 3E8 mouse monoclonal antibody) or 1:2000 (mouse monoclonal antibodies 3F11 and 2F7). Secondary EnVision HRP-conjugated goat anti-rabbit/mouse antibody (EV-GαM HRP, DAKO) was added for 30 min at room temperature followed by the chromogen 3,3,-Diaminobenzidine (DAKO) for 10 minutes. Sections were counterstained with haematoxylin).

Staining of brain sections from a frontotemporal lobar degeneration (FTLD) type B patient was performed. The rabbit polyclonal GS240 antibody shows staining of pathologic TDP43 in both white matter (WM) and grey matter (GM). The 3F11 and 3E8 monoclonal antibodies primarily detect pathologic TDP-43 in GM while the 2F7 monoclonal antibody shows staining in both WM and GM. The rabbit polyclonal GS240 antibody was also tested on ALS spinal cord sections and showed positivity in motor neurons and surrounding tissues. These results indicate that the antibodies are able to recognize epitopes of pathogenic TDP-43 as presented in situ in diseased tissues.

### Example 8

### Representative Staining of Transfected HEK293 Cells by Monoclonal Anti-TDP43 Antibody

Mouse and rabbit monoclonal antibodies were tested for selective binding to misfolded TDP-43 in a cell transfection assay using immunohistochemistry as described in Example 1. Briefly, HEK293FT cells were transfected with plasmid encoding either HA-tagged ΔNLS-TDP43 (which forms cytoplasmic aggregates) or HA-tagged wild type (WT) TDP-43 (expressed in the nucleus) or empty vector. The anti-TDP-43 antibodies were diluted to 10 ug/ml for staining and fluorescently-labeled anti-rabbit IgG or anti-mouse IgG were used as secondary antibodies for detection. A chicken anti-HA tag antibody followed by a fluorescently labeled anti-chicken secondary antibody was used for detection of transfected TDP-43. Transfected ΔNLS-TDP43 mislocalizes to the cytoplasm where it forms aggregates that are readily detected by staining with the anti-HA tag antibody. The 2F7 antibody recognized the same aggregates as confirmed by co-localization of the 2 staining signals in the merged images. In contrast, transfected WT TDP43, which is detected in the nucleus by the HA tag antibody, was not stained by the 2F7 antibody. As expected, cells transfected with empty vector do not show any HA tag staining. They also do not show any staining by the 2F7 antibody indicating that 2F7 also fails to recognize endogenous, nuclear WT TDP-43.

Similar results were obtained with other antibodies tested and are summarized in Table 6. This pattern of staining by the antibodies tested demonstrates their selectivity for misfolded, pathogenic aggregates of TDP-43 vs WT TDP-43.

TABLE 6
Binding to TDP-43 aggregates and WT TDP-43

| | | |
|---|---|---|
| Monoclonal | Binding to ΔNLS TDP-43 | Binding to nuclear |
| antibody | cytoplasmic aggregates | WT TDP-43 |

Mouse

| | | |
|---|---|---|
| 2F7 | Yes | No |
| 3E8 | Yes | No |
| 3F11 | Yes | No |

Rabbit

| | | |
|---|---|---|
| 1 (1H3-1K3) | Yes | No |
| 14 (14H1-14K2) | Yes | No |
| 17 (17H3-17K3) | Yes | No |
| 20 (20H2.2-20K1) | Yes | No |
| 28 (28H3-28K1) | Yes | No |
| 30 (30H3-30K1) | Yes | No |
| 36 (36H3-36K2) | Yes | No |
| 38 (38H1-38K1) | Yes | No |

### Example 9

### Immunocytochemistry of Physiologic Stress Granules

To determine whether the monoclonal antibodies to misfolded TDP-43 reacted with physiologic stress granules, staining was performed on stressed HEK293FT cells. Briefly, HEK293FT cells were stressed by exposure to 1 nM sodium arsenite for 60 min. The cells were then stained with fluorescently labeled antibody against the stress granule marker G3BP1 or with monoclonal anti-TDP-43 test antibodies at 10 ug/ml followed by detection with labeled secondary antibody. With the exception of one antibody, similar results were obtained with other antibodies tested and are included in Table 7 below. Stressed cells showed abundant punctate staining of G3BP1+ stress granules in the cytoplasm. By comparison, the same cells stained with 3F11 antibody did not show the presence of cytoplasmic granules in locations with G3BP1 staining indicating that 3F11 does not react with the stress granules in these cells. The lack of binding of the antibodies tested to TDP-43 in physiologic stress granules suggests that they are unlikely to interfere with the protective function of these stress granules.

TABLE 7
Binding of antibodies to stress granules

| | |
|---|---|
| Monoclonal | Binding to stress |
| antibody | granules |

Mouse

| | |
|---|---|
| 2F7 | No |
| 3E8 | No |
| 3F11 | No |

Rabbit

| | |
|---|---|
| 1 (1H3-1K3) | No |
| 14 (14H1-14K2) | No |
| 17 (17H3-17K3) | No |
| 20 (20H2.2-20K1) | Yes |
| 28H3-28K1 | No |
| 30 (30H3-30K1) | No |
| 36 (36H3-36K2) | No |
| 38 (38H1-38K1) | No |

### Example 10

Characterization of Selected mAbs

Monoclonal antibodies were tested for recognition of cytoplasmic aggregates formed in HEK293FT cells transfected with HA-tagged ΔNLS-TDP-43 vs HA-tagged ΔNLS-TDP-43 in which tryptophan 68 (Trp68) was mutated to serine (W68S). Empty vector was used as a control. Cells were stained with either the TDP-43 rabbit monoclonal antibody 28 at 2 pg/ml, mouse pan-TDP43 at 1 pg/ml or chicken anti-HA tag at 0.5 pg/ml. Fluorescently-labeled secondary antibodies Alexa Fluor 488-anti-rabbit, Alexa Fluor 647 anti-mouse and Alexa Fluor 568-anti-chicken were used for detection of bound primary antibody. Nuclei were stained with Hoechst 33342 dye. In cells transfected with HA-tagged ΔNLS-TDP-43, the 28 antibody stained cytoplasmic aggregates that co-localized with HA-positive aggregates of misfolded ΔNLS-TDP-43. In contrast, there was no staining of cytoplasmic aggregates formed by ΔNLS-TDP-43-W68S lacking Trp68. As expected, cells transfected with empty vector did not show any HA tag staining. They also did not show any staining by 28H3-28K1 confirming that the antibody does not recognize endogenous, nuclear WT TDP-43. The pan-TDP-43 antibody recognized cytoplasmic aggregates formed by both forms of transfected TDP-43 as well as endogenous nuclear TDP-43.

Similar results were obtained with most other antibodies tested and are summarized in Table 8. As observed with polyclonal rabbit antibody, this pattern of staining by the monoclonal antibodies tested demonstrates their selectivity for misfolded TDP-43 NTD comprising solvent-exposed Trp68 residue. Clone 20, bound some aggregates of ΔNLS-TDP-43-W68S, and bound to physiological stress granules.

TABLE 8
Summary of binding data for antibodies

| | Binding to | | | |
|---|---|---|---|---|
| | ΔNLS | | | |
| | TDP-43 | Binding to | Binding | Requires |
| | cytoplasmic | nuclear WT | to stress | TRP68 for |
| Antibody | aggregates | TDP43 | granules | binding* |
| Mouse | | | | |
| 2F7 | Yes | No | No | Yes |
| 3E8 | Yes | No | No | Not |
| | | | | determined |
| 3F11 | Yes | No | No | Not |
| | | | | Determined |
| Rabbit | | | | |
| 1 (1H3-1K3) | Yes | No | No | Yes |
| 14 (14H1-14K2) | Yes | No | No | Yes |
| 17 (17H3-17K3) | Yes | No | No | Yes |
| 20 (20H2.2-20K1) | Yes | No | Yes | +/− (binds |
| | | | | some W68S |
| | | | | aggregates) |
| 28 (28H3-28K1) | Yes | No | No | Yes |
| 30 (30H3-30K1) | Yes | No | No | Yes |
| 36 (36H3-36K2) | Yes | No | No | Yes |
| 38 (38H1-38K1) | Yes | No | No | Not |
| | | | | determined |

*Does not stain aggregates in cells transduced with ΔNLS-W68S TDP43 where tryptophan 68 is mutated to a serine residue

### Example 11

### Antibody Blocking of Misfolded TDP-43 Transmission in HEK293 Cells.

Donor HEK293 cells were transiently transfected with an HA-tagged nuclear-localization signal defective mutant of TDP-43, HA-ΔNLS-TDP43, to express misfolded TDP-43. 48 h post transfection, conditioned medium was collected from donor cells, and centrifuged at 1,000 g for 10 min to remove floating cell debris from the medium. Clarified conditioned media were incubated with 30 ug/ml of each individual TDP-43 misfolding specific antibodies or control mouse IgG1 (Biogen) for 1 h at room temperature with constant rotation prior to adding it to naïve recipient HEK293 cells. The antibodies tested included three mouse monoclonal antibodies to the N-terminal epitope of TDP-43 (3F11, 2F7, 3E8) and an antibody (9C5) against a conformational RRM1 epitope (PCT CA/2018/050634 published as WO 2018/218352). After 48 h of incubation, recipient cell medium was removed, cells were washed with cold PBS twice, and lysed in 2% SDS. Protein concentrations were measured using a BCA assay. 25 ug of lysate was separated on a 10% NuPage gel (Thermo) and transferred onto a PVDF membrane, followed by Western blotting with antibodies against the HA tag (Abcam, rabbit, 1:1000) or GAPDH (Thermo, mouse, 1:50K) as a loading control. HA and GAPDH immuno-reactivity was detected on the ChemiDoc Imaging System, and intensity was quantitated using Image Lab.

Donor cells transfected with HA-dNLS-TDP43 contained high amounts of HA-tagged TDP-43. Naïve recipient cells incubated with donor cell supernatant treated with control mouse IgG1 (mIgG1) contained detectable amounts of HA-tagged TDP-43 indicating that misfolded aggregates of HA-dNLS-TDP-43 protein were transmitted extracellularly from donor cells to recipient cells. Recipient cells incubated with donor cell supernatant pre-treated with misfolding-specific TDP-43 antibodies contained relatively lower amounts of HA-tagged TDP-43 showing inhibition of transmission by the antibodies. As a negative control, recipient cells incubated with supernatant from untransfected donor cells did not contain any detectable HA-tagged TDP-43. For each antibody, the HA tag signal was first normalized to the GAPDH signal (HA intensity/GAPDH intensity) and the value obtained for the test antibody was divided by the value obtained with control mIgG1. Relative to control mIgG1, all antibodies tested inhibited transmission of misfolded HA-dNLS-TDP-43 from donor cell supernatant resulting in lower levels of HA-tagged TDP-43 in the recipient cells.

### Example 12

### Vector Generation

Various single chain variable fragment (scFv) intrabody constructs were designed using short and heavy chain variable regions from antibodies directed to misfolded TDP-43. The variable heavy chain (V_{H}) and variable light chain (V_{L}) regions from antibodies 2F7, 1H3-1 K3, 28H3-28K1, and 14H1-14K2 which bind an epitope comprising at least W68 in the context of DAGWGNL (SEQ ID NO: 1) in misfolded TDP-43 were connected using various amino acid linker. The scFv peptides were constructed with the general sequence of either V_{H}-linker-V_{L} or V_{L}-linker-V_{H}. FLAG or MYC tag regions were linked to the scFv in order to detect expression and localization of the intrabodies Example 13. Lysosomal targeting region YPTL was also linked to the scFv peptide of some intrabodies.

The construction of 2F7, 1H3-1K3, 28H3-28K1, and 14H1-14K2 are described herein.

Six different types of single chain antibody constructs were generated for each of the antibodies having the following structures:

(a) FLAG tag-V_{H}-(G45)₃ Linker-V_{L}-G45 linker—Lysosomal targeting tag constructs;

(b) V_{H}-(G45)3 Linker-V_{L}-FLAG tag—YPTLconstructs;

(c) V_{L} linked to a V_{H} through a 15 amino acid linker—MYC tag construct;

(d) V_{H} linked to a V_{L} through a 15 amino acid linker—MYC tag construct;

(e) V_{L} linked to a V_{H} through a 20 amino acid linker—MYC tag construct; and

(f) V_{H} linked to a V_{L} through a 20 amino acid linker—MYC tag construct.

The TDP-43 ScFv vector constructs were generated by cloning the polynucleotide sequence of each of the scFv constructs into an expression plasmid, such as pcDNA3.1 vector, for example pcDNA3.1(+) or pcDNA3.1(−).

The vector and the scFv insert were digested by the NheI and HindIII restriction enzymes. After enzyme digestion, the vector and the insert were linked by T4 polymerase. 1-2 uL of the ligation reaction was transformed into TOP10 competent cells. Individual bacterial colonies were picked and overnight cultures were grown for DNA purification. After DNA purification, a diagnostic restriction digestion of the purified DNA with NheI and HindIII was conducted. The final plasmid was sequenced.

a) N-Term FLAG Tag Lysosomal Constructs

The group a) scFv constructs comprising a lysosomal-targeting tag are identified as LYS-2F7, LYS-1H3-1 K3, LYS-28H3-28K1, and LYS-14H1-14K2. The amino acid and polynucleotide sequences for lysosomal-targeting constructs LYS-2F7, LYS-1H3-1K3, LYS-28H3-28K1, and LYS-14H1-14K2 are shown below in Table 9. The V_{H} and V_{L} regions for LYS-2F7, LYS-1H3-1K3, LYS-28H3-28K1, and LYS-14H1-14K2 are based on the corresponding V_{H} and V_{L} regions from antibodies 2F7, 1H3-1 K3, 28H3-28K1, and 14H1-14K2 respectively.

TABLE 9
Polynucleotide and amino acid sequences for intrabodies LYS-2F7, LYS-1H3-1K3, LYS-
28H3-28K1, and LYS-14H1-14K2. FLAG tag sequences are shown in underlined font, the linker
sequences are shown in bold font, and the lysosomal targeting sequences are shown shaded and
in italic font.

| Label | | |
|---|---|---|
| SEQ ID NO | Polynucleotide sequence | Amino acid sequence |
| LYS-2F7 | ATGGACTACAAGGACGACGACGACAAGGACGTGAAGCTGGTGGAAAGCGGAGGA | MDYKDDDDKDVKLVESG |
| 202, 203 | GGACTGGTGAAGCTGGGCGGAAGCCTGAAGCTGTCTTGCGCCGCCAGCGGCTTC | GGLVKLGGSLKLSCAASG |
| | ACCTTTAGCAGCTACTACATGTCTTGGGTCCGGCAGACCCCAGAGAAGAGACTGG | FTFSSYYMSWWRQTPEKR |
| | AGCTGGTGGCCACCATCAACAGCAACGGAGGCAGCACCTACTACCCCGATACCGT | LELVATINSNGGSTYYPDT |
| | GAAGGGCCGGATCACCATCAGCAGGGACAACGCCAAGAACACCCTGCAGCTGCA | VKGRITISRDNAKNTLQLQ |
| | GATGAGCAGCCTGAGAAGCGAGGACACCGCCCTGTACTATTGCGTGCGGCAGAAC | MSSLRSEDTALYYCVRQN |
| | TACGAGGGAGCCTATTGGGGACAGGGAACACTGGTGACAGTGTCCGCAGGAGGA | YEGAYWGQGTLVTVSAG |
| | GGAGGAAGCGGAGGAGGAGGAAGCGGAGGAGGAGGATCTGACGTGCTGATGACC | GGGSGGGGSGGGGSDVL |
| | CAGACCCCTCTGTCTCTGCCAGTGACACTGGGAGACCAGGCTTCTATCTCTTGCCG | MTQTPLSLPVTLGDQASIS |
| | GAGCAGCCAGAGCATCGTGCACAGCAACGGCAACACCTACCTCGAGTGGTACCTG | CRSSQSIVHSNGNTYLEW |
| | CAGAAGCCAGGACAGAGCCCCAAGCTGCTGATCTACAAGGTGTCCAACCGGTTCA | YLQKPGQSPKLLIYKVSNR |
| | GCGGCGTGCCAGATAGATTCAGCGGAAGCGGAAGCGGCACCGACTTCACCCTGA | FSGVPDRFSGSGSGTDFT |
| | AGATCTCTAGAGTGGAAGCCGAGGATCTGGGCGTGTACTACTGCTTCCAGAGCAG | LKISRVEAEDLGVYYCFQS |
| | CCACGTGCCTTGGACATTTGGCGGCGGCACAAAGCTGGAGATCAAGGGCGGCGG | SHVPWTFGGGTKLEIKGG |
| | CGGCAGCAAGAGCATCCGGAGCGGCTACGAGGTCATGTGATAA | GGSKSIRSGYEVM- |
| LYS-1H3- | ATGGACTACAAGGACGACGACGACAAGCAGAGCGTGGAGGAAAGCGGAGGAAGA | MDYKDDDDKQSVEESGG |
| 1K3 | CTGGTGACACCAGGCACCCCTCTGACCCTGACTTGTACAGTGTCCGGCTTCAGCC | RLVTPGTPLTLTCTVSGFS |
| 204, 205 | TGAGCAGGTACTACATGACTTGGGTCCGGCAGGCACCAGGAAAGGGCCTGGAGTA | LSRYYMTWWRQAPGKGL |
| | CATCGGCGTGATCATCCCAGGAGGCACCACATACTACGCCTCTTGGGCCAAGGGC | EYIGVIIPGGTTYYASWAK |
| | AGGTTCACCATCAGCAAGACCAGCACCACCGTGGACCTGAGGATCACAAGCCCTA | GRFTISKTSTTVDLRITSPT |
| | CCACCGAGGACACCGCCACCTACTTTTGTGCCGGAGGACCTACAGGCAATAGCCA | TEDTATYFCAGGPTGNSH |
| | CTTCACCCTCTGGGGACAGGGAACACTGGTGACAGTGTCCGGAGGAGGAGGAAG | FTLWGQGTLVTVSGGGG |
| | CGGAGGAGGAGGAAGCGGAGGAGGAGGAAGCGTGATGACACAGACCCCTAGCAG | SGGGGSGGGGSVMTQTP |
| | CAAGAGCGTGCCAGTGGGCGGAACCGTGACCATCAATTGCCAGGCCAGCGAGAG | SSKSVPVGGTVTINCQAS |
| | CGTGTACAACAACAACCACCTGTCTTGGTACCAGCAGAAGAGCGGCCAGCCTCCT | ESVYNNNHLSWYQQKSG |
| | AAGCTGCTGATCTACGAGGCCAGCAAGCTGGAGAGCGGAGTGCCTCCCAGATTCA | QPPKLLIYEASKLESGVPP |
| | AAGGCAGCGGAAGCGGCACCCAGTTTACCCTGACCATCTCAGACGTCGTCTGCGA | RFKGSGSGTQFTLTISDW |
| | CGACGCCGCTACCTACTATTGCAGCGGCTACAAGAGAGTGACCACCGACGGCATC | CDDAATYYCSGYKRVTTD |
| | GCCTTTGGAGGAGGCACAGAAGTGGTGGTGAAAGGCGGAGGAGGCAGCAAGAGC | GIAFGGGTEVVVKGGGGS |
| | ATCAGAAGCGGCTACGAGGTCATGTGATAA | KSIRSGYEVM- |
| LYS-28H3- | ATGGACTACAAGGACGACGACGACAAGCAGAGCCTGGAGGAAAGCGGAGGAAGA | MDYKDDDDKQSLEESGG |
| 28K1 | CTGGTGACACCAGGCACCCCTCTGACCCTGACTTGTACAGTGTCCGGCTTCAGCC | RLVTPGTPLTLTCTVSGFS |
| 206, 207 | TGAGCAGCTACAACATGGGTTGGGTGCGACAGGCTCCAGGAGAAGGACTCGAGTG | LSSYNMGWVRQAPGEGL |
| | GATCGGCGTGATCGGAACAGGAGGCATCACCCACTACGCCACTTGGGCTAAGGGC | EWIGVIGTGGITHYATWAK |
| | AGAGTGGCCATCAGCAGAACCAGCACCACCGTGGGACTGAGAATGACCAGCCCTA | GRVAISRTSTTVGLRMTSP |
| | CCACCGAGGACACCGCCACCTACTTTTGCGTGAGATCTAGCGGCAGCGATTGGTG | TTEDTATYFCVRSSGSDW |
| | GTTCCACATTTGGGGGCAGGGCACACTGGTGACAGTGTCCGGAGGAGGAGGAAG | WFHIWGQGTLVTVSGGG |
| | CGGAGGAGGAGGATCTGGAGGAGGAGGAAGCGTGCTCACACAGACAACAAGCCC | GSGGGGSGGGGSVLTQT |
| | AGTGTCCGCCGCAGTGGGAGGAACCGTGACCATCTCTTGCCAGAGCAGCCAGAG | TSPVSAAVGGTVTISCQSS |
| | CGTGTACAACAACAACAACCTGGCTTGGTTCCAGCAGAAGCCCGGACAGCCTCCT | QSVYNNNNLAWFQQKPG |
| | AAGCTGCTGATCTACAGGGCCAGCAACCTGCCTAGCGGCGTGCCTAGCAGATTCA | QPPKLLIYRASNLPSGVPS |
| | GAGGAAGCGGAAGCGGCAGCCAGTTTACCCTGACCATCAGCGAAGTCCAGTGCGA | RFRGSGSGSQFTLTISEV |
| | CGACGCAGCCACCTACTATTGCCAGGGCTACTTCAGCGGCTTTATCACCACCTTTG | QCDDAATYYCQGYFSGFI |
| | GCGGCGGAACCGAAGTGGTGGTGAAAGGCGGAGGAGGAAGCAAGAGCATCCGGA | TTFGGGTEVWKGGGGSK |
| | GCGGCTACGAAGTGATGTGATAA | SIRSGYEVM- |
| LYS-14H1- | ATGGACTACAAGGACGACGACGACAAGCAGGAGCAGCTGGAAGAGAGCGGAGGA | MDYKDDDDKQEQLEESG |
| 14K2 | GATCTGGTGAAGCCAGAGGGCAGCCTGACACTGACTTGTACAGCCAGCGGCTTCA | GDLVKPEGSLTLTCTASGF |
| 208, 209 | GCTTCAGCAGCAACTACGTCATGTGTTGGGTCCGGCAGGCACCAGGAAAAGGACT | SFSSNYVMCWVRQAPGK |
| | GGAGTGGGTGGCTTGCATTTGGTTCGCCGGCATCGTGGACACCACCTACTACGCC | GLEWWACIWFAGIVDTTYY |
| | ACTTGGGCCAAGGGACGGTTCACCATCAGCAAGACCAGCAGCACCACCGTGACCC | ATWAKGRFTISKTSSTTVT |
| | TGCAGATGACATCTCTGACAGCCGCCGACACCGCTACCTACTTTTGCGCCCGGAA | LQMTSLTAADTATYFCAR |
| | CCCAGTGGGATCAGTCAACCTCTGGGGACAGGGAACACTGGTGACCGTGTCAGGA | NPVGSVNLWGQGTLVTVS |
| | GGAGGAGGATCTGGAGGAGGAGGAAGCGGAGGAGGAGGCAGCGTGATGACACA | GGGGSGGGGSGGGGSV |
| | GACACCTAGCAGCAAAAGCGTGCCAGTGGGAGGAAGCGTGACCATCAATTGCCAG | MTQTPSSKSVPVGGSVTI |
| | GCCAGCGAGAGCGTGTACAGCAACAACCGGCTGTCTTGGTACCAGCAGAAGCCAG | NCQASESVYSNNRLSWY |
| | GCCAGCCTCCTAAGCTGCTGATCTACTACGCCTCTACCCTGGAGAGCGGAGTGCC | QQKPGQPPKLLIYYASTLE |
| | TAGCAGATTCAAGGGCAGCGGCTTTGGCACCCACTTCACACTGACCATCAGCGGA | SGVPSRFKGSGFGTHFTL |
| | GCCCAGTGCGACGACGCAGCCACCTACTATTGCGCAGGCTGGAGAGGAGCTAGA | TISGAQCDDAATYYCAGW |
| | ACAGACGGAGTGGACTTCGGCGGAGGAACAGAAGTGGTGGTGAAAGGCGGCGGC | RGARTDGVDFGGGTEVV |
| | GGCTCTAAAAGCATCCGGAGCGGCTACGAGGTCATGTGATAA | VKGGGGSKSIRSGYEVM- |

b) C-Term FLAG—YPTL Constructs

The group b) YPTL comprising constructs are identified as YPTL-2F7, YPTL-1H3-1 K3, YPTL-28H3-28K1, and YPTL-14H1-14K2. The amino acid and polynucleotide sequences for constructs YPTL-2F7, YPTL-1H3-1K3, YPTL-28H3-28K1, and YPTL-14H1-14K2 are shown below in Table 10. The V_{H} and V_{L} regions for YPTL-2F7, YPTL-1H3-1K3, YPTL-28H3-28K1, and YPTL-14H1-14K2 are based on the corresponding V_{H} and V_{L} regions from antibodies 2F7, 1H3-1K3, 28H3-28K1, and 14H1-14K2 respectively.

TABLE 10
Polynucleotide and amino acid sequences for intrabodies YPTL-2F7, YPTL-1H3-1K3,
YPTL-28H3-28K1, and YPTL-14H1-14K2. FLAG tag sequences are shown in underlined font, the linker
sequences are shown in bold font, and the lysosomal targeting sequences are shown shaded and
in italic font.

| Label | | |
|---|---|---|
| SEQ ID NO | Polynucleotide sequence | Amino Acid Sequence |
| YPTL-2F7 | atgGACGTGAAGCTCGTGGAGTCTGGGGGAGGCTTAGTGAAGCTTGGAGG | MDVKLVESGGGLVKLGGSLKL |
| 212, 213 | GTCCCTGAAACTCTCCTGTGCAGCCTCTGGATTCACTTTCAGTAGCTATTA | SCAASGFTFSSYYMSWVRQTP |
| | CATGTCTTGGGTTCGCCAGACTCCAGAGAAGAGGCTGGAGTTGGTCGCA | EKRLELVATINSNGGSTYYPDT |
| | ACCATTAATAGTAATGGTGGTAGCACCTACTATCCAGACACTGTGAAGGG | VKGRITISRDNAKNTLQLQMSS |
| | CCGAATCACCATCTCCAGAGACAATGCCAAGAACACCCTGCAGTTGCAAA | LRSEDTALYYCVRQNYEGAYW |
| | TGAGCAGTCTGAGGTCTGAGGACACAGCCTTGTATTACTGTGTAAGACAA | GQGTLVTVSAGGGGSGGGGS |
| | AACTACGAGGGGGCTTACTGGGGCCAAGGGACTCTGGTCACTGTCTCTG | GGGGSDVLMTQTPLSLPVTLG |
| | CAGGCGGAGGTGGTAGCGGAGGAGGTGGCAGCGGGGGAGGTGGCTCA | DQASISCRSSQSIVHSNGNTYL |
| | GATGTTTTGATGACCCAAACTCCACTCTCCCTGCCTGTCACTCTTGGAGAT | EWYLQKPGQSPKLLIYKVSNRF |
| | CAAGCCTCCATCTCTTGCAGATCTAGTCAGAGCATTGTACATAGTAATGGA | SGVPDRFSGSGSGTDFTLKISR |
| | AACACCTATTTAGAATGGTACCTGCAGAAACCAGGCCAGTCTCCAAAGCT | VEAEDLGVYYCFQSSHVPWTF |
| | CCTGATCTACAAAGTTTCCAACCGATTTTCTGGGGTCCCAGACAGGTTCA | GGGTKLEIKDYKDDDDKRWRK |
| | GTGGCAGTGGATCAGGGACAGATTTCACACTCAAGATCAGCAGAGTGGA | SHSSSYTPLSGSTYPEGRH- |
| | GGCTGAGGATCTGGGAGTTTATTACTGCTTTCAAAGTTCACATGTTCCGTG | |
| | GACGTTCGGTGGAGGCACCAAGCTGGAAATCAAAGATTACAAGGATGACG | |
| | ACGATAAGCGCTGGCGTAAATCTCATTCATCCTCCTACACCCCCTTGTCC | |
| | GGTTCTACCTACCCCGAAGGGCGCCATtag | |
| YPTL-1H3-1K3 | atgCAGTCGGTGGAGGAGTCCGGGGGTCGCCTGGTCACGCCTGGGACAC | MQSVEESGGRLVTPGTPLTLT |
| 214, 215 | CCCTGACACTCACCTGCACAGTCTCTGGATTCTCCCTCAGTAGGTACTAC | CTVSGFSLSRYYMTWVRQAPG |
| | ATGACCTGGGTCCGCCAGGCTCCAGGGAAGGGGCTGGAATACATCGGGG | KGLEYIGVIIPGGTTYYASWAKG |
| | TCATTATTCCTGGTGGTACCACATACTACGCGAGCTGGGCGAAAGGCCGA | RFTISKTSTTVDLRITSPTTEDT |
| | TTCACCATCTCCAAAACCTCGACCACGGTGGATCTGAGAATCACCAGTCC | ATYFCAGGPTGNSHFTLWGQG |
| | GACAACCGAGGACACGGCCACTTATTTCTGTGCCGGAGGTCCTACTGGTA | TLVTVSSGGGGSGGGGSGGG |
| | ACAGCCACTTTACATTGTGGGGCCAGGGCACCCTGGTCACCGTCTCCTCA | GSVMTQTPSSKSVPVGGTVTIN |
| | GGCGGAGGTGGTAGCGGAGGAGGTGGCAGCGGGGGAGGTGGCTCAGT | CQASESVYNNNHLSWYQQKS |
| | GATGACCCAGACTCCATCTTCCAAGTCTGTCCCTGTGGGAGGCACAGTCA | GQPPKLLIYEASKLESGVPPRF |
| | CCATCAATTGCCAGGCCAGTGAGAGTGTTTATAATAACAACCACTTATCCT | KGSGSGTQFTLTISDVVCDDAA |
| | GGTATCAGCAGAAATCAGGGCAGCCTCCCAAGCTCCTGATCTACGAAGCA | TYYCSGYKRVTTDGIAFGGGTE |
| | TCCAAACTGGAATCTGGGGTCCCACCGCGGTTCAAAGGCAGTGGATCTG | VVKDYKDDDDKRWRKSHSSS |
| | GGACACAGTTCACTCTCACCATCAGCGATGTGGTGTGTGACGATGCTGCC | YTPLSGSTYPEGRH- |
| | ACTTACTACTGTTCAGGATATAAACGTGTTACTACTGATGGTATTGCTTTCG | |
| | GCGGAGGGACCGAGGTGGTGGTCAAAGATTACAAGGATGACGACGATAA | |
| | GCGCTGGCGTAAATCTCATTCATCCTCCTACACCCCCTTGTCCGGTTCTAC | |
| | CTACCCCGAAGGGCGCCATtag | |
| YPTL-28H3- | atgCAGTCGCTGGAGGAGTCCGGGGGTCGCCTGGTCACGCCTGGGACAC | MQSLEESGGRLVTPGTPLTLTC |
| 28K1 | CCCTGACACTCACCTGCACAGTCTCTGGATTCTCCCTCAGTAGCTACAAC | TVSGFSLSSYNMGWVRQAPGE |
| 216, 217 | ATGGGCTGGGTCCGCCAGGCTCCAGGGGAGGGGCTGGAGTGGATCGGA | GLEWIGVIGTGGITHYATWAKG |
| | GTCATTGGTACTGGTGGTATCACACACTACGCGACCTGGGCAAAAGGCCG | RVAISRTSTTVGLRMTSPTTED |
| | AGTCGCCATCTCCAGAACCTCGACCACGGTGGGTCTGCGAATGACCAGT | TATYFCVRSSGSDWWFHIWGQ |
| | CCGACAACCGAGGACACGGCCACCTATTTCTGTGTCAGATCTAGTGGTAG | GTLVTVSSGGGGSGGGGSGG |
| | TGATTGGTGGTTTCACATCTGGGGCCAGGGCACCCTGGTCACCGTCTCCT | GGSVLTQTTSPVSAAVGGTVTI |
| | CAGGCGGAGGTGGTAGCGGAGGAGGTGGCAGCGGGGGAGGTGGCTCA | SCQSSQSVYNNNNLAWFQQK |
| | GTGCTGACCCAGACTACATCGCCCGTGTCTGCAGCTGTGGGAGGCACAG | PGQPPKLLIYRASNLPSGVPSR |
| | TCACCATCAGTTGCCAGTCCAGTCAGAGTGTTTATAATAACAACAACTTAG | FRGSGSGSQFTLTISEVQCDDA |
| | CCTGGTTTCAGCAGAAACCAGGGCAGCCTCCCAAGCTCCTGATCTACAGG | ATYYCQGYFSGFITTFGGGTEV |
| | GCATCCAATCTGCCATCTGGTGTCCCATCGCGGTTCAGAGGCAGTGGATC | WKDYKDDDDKRWRKSHSSSY |
| | TGGGTCACAGTTCACTCTCACAATCAGCGAAGTACAGTGTGACGATGCTG | TPLSGSTYPEGRH- |
| | CCACTTACTACTGTCAAGGCTATTTTAGTGGATTTATCACTACTTTCGGCG | |
| | GAGGGACCGAGGTGGTGGTCAAAGATTACAAGGATGACGACGATAAGCG | |
| | CTGGCGTAAATCTCATTCATCCTCCTACACCCCCTTGTCCGGTTCTACCTA | |
| | CCCCGAAGGGCGCCATtag | |
| YPTL-14H1- | atgCAGGAGCAGCTGGAGGAGTCCGGGGGAGACCTGGTCAAGCCTGAGG | MQEQLEESGGDLVKPEGSLTL |
| 14K2 | GATCCCTGACACTCACCTGCACAGCCTCTGGATTCTCCTTCAGTAGCAAC | TCTASGFSFSSNYVMCWWRQA |
| 218, 219 | TACGTGATGTGCTGGGTCCGCCAGGCTCCAGGGAAGGGGCTGGAGTGG | PGKGLEWVACIWFAGIVDTTYY |
| | GTCGCATGCATTTGGTTTGCTGGTATTGTTGATACTACTTACTACGCGACC | ATWAKGRFTISKTSSTTVTLQM |
| | TGGGCGAAAGGCCGATTCACCATCTCCAAAACCTCGTCGACCACGGTGAC | TSLTAADTATYFCARNPVGSVN |
| | TCTGCAAATGACCAGTCTGACAGCCGCGGACACGGCCACCTATTTCTGTG | LWGQGTLVTVSSGGGGSGGG |
| | CGAGAAATCCTGTTGGTAGTGTGAACTTGTGGGGCCAGGGCACCCTGGT | GSGGGGSVMTQTPSSKSVPV |
| | CACCGTCTCCTCAGGCGGAGGTGGTAGCGGAGGAGGTGGCAGCGGGG | GGSVTINCQASESVYSNNRLS |
| | GAGGTGGCTCAGTGATGACCCAGACTCCATCTTCCAAGTCTGTCCCTGTG | WYQQKPGQPPKLLIYYASTLES |
| | GGAGGCTCAGTCACCATCAATTGCCAGGCCAGTGAGAGTGTTTATAGTAA | GVPSRFKGSGFGTHFTLTISGA |
| | CAACCGCTTATCCTGGTATCAGCAGAAACCAGGGCAGCCTCCTAAGCTCC | QCDDAATYYCAGWRGARTDG |
| | TGATCTATTATGCATCCACTCTGGAATCTGGGGTCCCATCGCGGTTCAAA | VDFGGGTEVVVKDYKDDDDKR |
| | GGCAGTGGATTTGGGACACACTTCACTCTCACCATCAGCGGCGCGCAGT | WRKSHSSSYTPLSGSTYPEGR |
| | GTGACGATGCTGCCACTTACTACTGTGCAGGATGGAGAGGTGCTAGGACT | H- |
| | GATGGTGTAGATTTCGGCGGAGGGACCGAGGTGGTGGTCAAAGATTACA | |
| | AGGATGACGACGATAAGCGCTGGCGTAAATCTCATTCATCCTCCTACACC | |
| | CCCTTGTCCGGTTCTACCTACCCCGAAGGGCGCCATtag | |

c) C-Terminal MYC Tag Constructs with a V_{L} Linked to a V_{H} Through a 15 Amino Acid Linker:

The MYC tag constructs with a V_{L} linked to a V_{H} through a 15 amino acid linker are, MYCL15H-2F7, MYCL15H-1H3-1K3, MYCL15H-28H3-28K1, and MYCL15H-14H1-14K2. These constructs possess the general sequences of V_{L}—15 amino acid ScFv linker sequence—V_{H}—three amino acid MYC linker sequence—MYC Tag. The amino acid and polynucleotide sequences for constructs MYCL15H-2F7, MYCL15H-1H3-1K3, MYCL15H-28H3-28K1, and MYCL15H-14H1-14K2 are shown below in Table 11. The V_{H} and V_{L} regions for MYCL15H-2F7, MYCL15H-1H3-1K3, MYCL15H-28H3-28K1, and MYCL15H-14H1-14K2 are based on the corresponding V_{H} and V_{L} regions from antibodies 2F7, 1H3-1K3, 28H3-28K1, and 14H1-14K2 respectively.

TABLE 11
Polynucleotide and amino acid sequences for intrabodies MYCL15H-2F7, MYCL15H-1H3-
1K3, MYCL15H-28H3-28K1, and MYCL15H-14H1-14K2. The MYC tag sequences are shown in
underlined font and the linker sequences is shown in bold font.

| Label | | |
|---|---|---|
| SEQ ID NO | Polynucleotide sequence | Amino Acid Sequence |
| MYCL15H-2F7 | atgGATGTGCTCATGACACAAACACCTTTGAGCCTTCCCGTGACACTCGG | MDVLMTQTPLSLPVTLGDQASIS |
| 222, 223 | CGACCAGGCCAGTATCTCCTGCAGAAGTTCCCAGTCTATCGTGCACTCA | CRSSQSIVHSNGNTYLEWYLQK |
| | AACGGTAATACGTACTTGGAGTGGTATCTGCAAAAACCCGGACAGTCCC | PGQSPKLLIYKVSNRFSGVPDRF |
| | CAAAGCTGTTGATATACAAGGTTTCTAATCGCTTCAGCGGTGTACCAGAC | SGSGSGTDFTLKISRVEAEDLGV |
| | AGATTTTCTGGCTCCGGATCAGGAACCGACTTTACCCTTAAGATTTCAAG | YYCFQSSHVPWTGGGTKLEIKG |
| | GGTGGAAGCCGAGGACCTTGGAGTGTACTATTGCTTCCAATCAAGTCAC | GGGSGGGGSGGGGSDVKLVES |
| | GTCCCATGGACTGGGGGCGGCACCAAGCTCGAGATCAAGGGAGGTGG | GGGLVKLGGSLKLSCAASGFTFS |
| | CGGATCAGGTGGCGGAGGTTCTGGCGGTGGCGGTTCTGATGTGAAGTT | SYYMSWVRQTPEKRLELVATINS |
| | GGTTGAAAGTGGAGGCGGTCTCGTTAAACTGGGCGGAAGTCTGAAACTT | NGGSTYYPDTVKGRITISRDNAK |
| | TCATGTGCAGCTTCCGGGTTCACCTTCTCTAGCTATTACATGAGTTGGGT | NTLQLQMSSLRSEDTALYYCVR |
| | ACGGCAGACCCCCGAAAAGCGACTGGAGCTGGTGGCTACCATAAATTCT | QNYEGAYWGQGTLVTVSAGSG |
| | AACGGAGGATCTACGTATTACCCCGACACCGTGAAGGGGCGCATCACC | EQKLISEEDL- |
| | ATTTCTCGGGACAATGCCAAAAATACACTGCAGTTGCAGATGTCATCACT | |
| | GCGAAGCGAAGACACCGCGCTGTACTACTGTGTAAGACAGAATTACGAG | |
| | GGCGCATATTGGGGACAGGGCACTCTGGTCACCGTGTCAGCTGGCAGT | |
| | GGAGAGCAGAAGCTGATCTCAGAGGAGGACCTGtga | |
| MYCL15H-1H3- | atgGCAATCGTGATGACCCAGACTCCTAGCTCCAAAAGCGTGCCCGTGG | MAIVMTQTPSSKSVPVGGTVTIN |
| 1K3 | GTGGAACTGTTACGATCAACTGTCAGGCGAGCGAGTCCGTGTACAACAA | CQASESVYNNNHLSWYQQKSG |
| 224, 225 | CAACCATCTTTCTTGGTATCAGCAGAAGTCTGGACAGCCGCCCAAGCTG | QPPKLLIYEASKLESGVPPRFKG |
| | CTCATATACGAAGCGTCAAAGCTGGAATCCGGAGTGCCACCTCGCTTTA | SGSGTQFTLTISDWCDDAATYY |
| | AGGGAAGCGGGAGTGGCACCCAGTTCACTCTGACCATAAGTGACGTTGT | CSGYKRVTTDGIAFGGGTEVVVK |
| | TTGTGACGACGCTGCCACATATTATTGCAGTGGTTATAAGCGAGTCACCA | GGGGSGGGGSGGGGSQSVEES |
| | CGGACGGCATCGCCTTTGGAGGTGGCACCGAGGTGGTTGTGAAGGGAG | GGRLVTPGTPLTLTCTVSGFSLS |
| | GTGGCGGATCAGGTGGCGGAGGTTCTGGCGGTGGCGGTTCTCAGAGC | RYYMTWVRQAPGKGLEYIGVIIP |
| | GTGGAGGAATCAGGTGGAAGACTCGTTACTCCAGGTACTCCGCTGACCT | GGTTYYASWAKGRFTISKTSTTV |
| | TGACTTGTACCGTGAGTGGCTTTTCCCTGTCCCGCTACTACATGACCTG | DLRITSPTTEDTATYFCAGGPTG |
| | GGTGAGGCAGGCACCTGGCAAAGGGCTGGAATACATTGGGGTGATCAT | NSHFTLWGQGTLVTVSSGSGEQ |
| | ACCTGGCGGAACAACATATTACGCCAGTTGGGCAAAAGGTCGCTTTACC | KLISEEDL- |
| | ATTTCCAAAACATCCACTACTGTGGACCTCAGAATCACATCCCCAACTAC | |
| | AGAAGACACAGCCACATACTTCTGCGCAGGAGGACCAACCGGCAACTCT | |
| | CATTTCACACTCTGGGGCCAGGGCACTCTGGTCACCGTGTCATCTGGCA | |
| | GTGGAGAGCAGAAGCTGATCTCAGAGGAGGACCTGtga | |
| MYCL15H- | atgGCACAAGTGTTGACCCAGACTACTTCACCCGTTAGTGCAGCCGTGGG | MAQVLTQTTSPVSAAVGGTVTIS |
| 28H3-28K1 | AGGCACCGTTACCATTAGCTGCCAGTCATCCCAGTCAGTTTATAATAACA | CQSSQSVYNNNNLAWFQQKPG |
| 226, 227 | ATAACCTTGCCTGGTTCCAGCAGAAACCAGGCCAGCCCCCCAAGCTCCT | QPPKLLIYRASNLPSGVPSRFRG |
| | GATCTATAGAGCCAGCAATCTGCCGAGCGGGGTTCCTTCCAGGTTCAGA | SGSGSQFTLTISEVQCDDAATYY |
| | GGGAGTGGTAGTGGGTCTCAGTTCACACTCACCATCAGCGAGGTGCAAT | CQGYFSGFITTFGGGTEVVVKG |
| | GTGATGACGCTGCTACCTACTACTGCCAGGGGTACTTCAGCGGATTCAT | GGGSGGGGSGGGGSQSLEESG |
| | CACAACCTTTGGAGGTGGCACCGAGGTGGTTGTGAAGGGAGGTGGCGG | GRLVTPGTPLTLTCTVSGFSLSS |
| | ATCAGGTGGCGGAGGTTCTGGCGGTGGCGGTTCTCAGAGCCTGGAGG | YNMGWVRQAPGEGLEWIGVIGT |
| | AATCAGGTGGAAGACTTGTTACACCAGGTACGCCCCTGACTCTGACATG | GGITHYATWAKGRVAISRTSTTV |
| | TACTGTGAGCGGATTTTCCCTGAGTAGCTATAACATGGGATGGGTCAGA | GLRMTSPTTEDTATYFCVRSSGS |
| | CAGGCCCCTGGTGAGGGTCTCGAGTGGATCGGTGTGATCGGTACCGGA | DWWFHIWGQGTLVTVSSGSGE |
| | GGTATCACTCACTATGCAACCTGGGCAAAAGGACGCGTGGCCATCAGCA | QKLISEEDL- |
| | GGACCAGCACAACTGTAGGCCTGCGGATGACTAGCCCAACAACCGAAG | |
| | ACACAGCAACATACTTTTGTGTCCGGAGCAGCGGTTCCGATTGGTGGTT | |
| | TCACATCTGGGGACAGGGAACTCTGGTCACCGTGTCATCTGGCAGTGG | |
| | AGAGCAGAAGCTGATCTCAGAGGAGGACCTGtga | |
| MYCL15H- | atgGCAATCGTGATGACCCAGACTCCTTCTAGTAAGAGTGTGCCTGTGGG | MAIVMTQTPSSKSVPVGGSVTIN |
| 14H1-14K2 | AGGGAGCGTGACAATCAACTGCCAGGCCAGCGAGTCCGTGTATAGTAAT | CQASESVYSNNRLSWYQQKPG |
| 228, 229 | AATCGACTCTCATGGTATCAACAGAAGCCCGGCCAGCCTCCTAAATTGTT | QPPKLLIYYASTLESGVPSRFKG |
| | GATATACTATGCAAGTACCCTCGAAAGCGGTGTTCCATCCAGGTTTAAAG | SGFGTHFTLTISGAQCDDAATYY |
| | GGAGCGGCTTTGGGACTCACTTCACTCTTACGATTAGTGGAGCACAATG | CAGWRGARTDGVDFGGGTEVV |
| | TGATGACGCAGCTACTTATTATTGTGCCGGTTGGAGAGGGGCTAGAACC | VKGGGGSGGGGSGGGGSQEQL |
| | GATGGAGTGGATTTTGGAGGTGGCACCGAGGTGGTTGTGAAGGGAGGT | EESGGDLVKPEGSLTLTCTASGF |
| | GGCGGATCAGGTGGCGGAGGTTCTGGCGGTGGCGGTTCTCAGGAACA | SFSSNYVMCWVRQAPGKGLEW |
| | ACTGGAGGAATCAGGTGGAGATTTGGTGAAGCCGGAGGGGTCTCTGAC | VACIWFAGIVDTTYYATWAKGRF |
| | CCTGACATGTACCGCCTCCGGGTTCAGTTTTTCCTCAAATTACGTCATGT | TISKTSSTTVTLQMTSLTAADTAT |
| | GCTGGGTCAGACAGGCGCCTGGAAAGGGGCTGGAATGGGTGGCATGC | YFCARNPVGSVNLWGQGTLVTV |
| | ATTTGGTTTGCTGGAATTGTAGATACCACCTACTATGCCACGTGGGCGAA | SSGSGEQKLISEEDL- |
| | AGGACGGTTCACCATCAGCAAGACTAGTAGCACAACTGTTACCCTCCAG | |
| | ATGACAAGCCTGACCGCAGCTGACACTGCCACTTATTTCTGCGCGCGCA | |
| | ACCCTGTTGGATCAGTTAATCTGTGGGGACAAGGCACTCTGGTCACCGT | |
| | GTCATCTGGCAGTGGAGAGCAGAAGCTGATCTCAGAGGAGGACCTGtga | |

d) MYC Constructs with a V_{H} Linked to a V_{L} Through a 15 Amino Acid Linker

The MYC constructs with a V_{H} linked to a V_{L} through a 15 amino acid linker are MYCH15L-2F7, MYCH15L-1H3-1K3, MYCH15L-28H3-28K1, and MYCH15L-14H1-14K2. All of these constructs possess the general sequences of V_{H}—15 amino acid ScFv linker sequence—V_{L}—three amino acid MYC linker sequence—MYC Tag. The amino acid and polynucleotide sequences for constructs MYCH15L-2F7, MYCH15L-1H3-1K3, MYCH15L-28H3-28K1, and MYCH15L-14H1-14K2 are shown below in Table 12, The V_{H} and V_{L} regions for, MYCH15L-2F7, MYCH15L-1H3-1K3, MYCH15L-28H3-28K1, and MYCH15L-14H1-14K2 are based on the corresponding V_{H} and V_{L} regions from antibodies 2F7, 1H3-1K3, 28H3-28K1, and 14H1-14K2 respectively.

TABLE 12
Polynucleotide and amino acid sequences for intrabodies MYCH15L-2F7, MYCH15L-1H3-
1K3, MYCH15L-28H3-28K1, and MYCH15L-14H1-14K2. The MYC tag sequences are shown in
underlined font and the linker sequences is shown in bold font.

| Label | | |
|---|---|---|
| SEQ ID NO | Polynucleotide sequence | Amino acid sequence |
| MYCH15L- | atgGATGTGAAGTTGGTTGAAAGTGGAGGCGGTCTCGTTAAACTGGGCGGAAGT | MDVKLVESGGGLVKLGGSLK |
| 2F7 | CTGAAACTTTCATGTGCAGCTTCCGGGTTCACCTTCTCTAGCTATTACATGAGTT | LSCAASGFTFSSYYMSWWR |
| 232, 233 | GGGTACGGCAGACCCCCGAAAAGCGACTGGAGCTGGTGGCTACCATAAATTCT | QTPEKRLELVATINSNGGST |
| | AACGGAGGATCTACGTATTACCCCGACACCGTGAAGGGGCGCATCACCATTTC | YYPDTVKGRITISRDNAKNTL |
| | TCGGGACAATGCCAAAAATACACTGCAGTTGCAGATGTCATCACTGCGAAGCG | QLQMSSLRSEDTALYYCVRQ |
| | AAGACACCGCGCTGTACTACTGTGTAAGACAGAATTACGAGGGCGCATATTGG | NYEGAYWGQGTLVTVSAGG |
| | GGACAGGGCACTCTGGTCACCGTGTCAGCTGGAGGTGGCGGATCAGGTGGC | GGSGGGGSGGGGSDVLMT |
| | GGAGGTTCTGGCGGTGGCGGTTCTGATGTGCTCATGACACAAACACCTTTGAG | QTPLSLPVTLGDQASISCRSS |
| | CCTTCCCGTGACACTCGGCGACCAGGCCAGTATCTCCTGCAGAAGTTCCCAGT | QSIVHSNGNTYLEWYLQKPG |
| | CTATCGTGCACTCAAACGGTAATACGTACTTGGAGTGGTATCTGCAAAAACCCG | QSPKLLIYKVSNRFSGVPDR |
| | GACAGTCCCCAAAGCTGTTGATATACAAGGTTTCTAATCGCTTCAGCGGTGTAC | FSGSGSGTDFTLKISRVEAE |
| | CAGACAGATTTTCTGGCTCCGGATCAGGAACCGACTTTACCCTTAAGATTTCAA | DLGVYYCFQSSHVPWTGGG |
| | GGGTGGAAGCCGAGGACCTTGGAGTGTACTATTGCTTCCAATCAAGTCACGTC | TKLEIKGSGEQKLISEEDL- |
| | CCATGGACTGGGGGCGGCACCAAGCTCGAGATCAAGGGCAGTGGAGAGCAG | |
| | AAGCTGATCTCAGAGGAGGACCTGtga | |
| MYCH15L- | atgCAGAGCGTGGAGGAATCAGGTGGAAGACTCGTTACTCCAGGTACTCCGCTG | MQSVEESGGRLVTPGTPLTL |
| 1H3-1K3 | ACCTTGACTTGTACCGTGAGTGGCTTTTCCCTGTCCCGCTACTACATGACCTGG | TCTVSGFSLSRYYMTWWRQ |
| 234, 235 | GTGAGGCAGGCACCTGGCAAAGGGCTGGAATACATTGGGGTGATCATACCTG | APGKGLEYIGVIIPGGTTYYA |
| | GCGGAACAACATATTACGCCAGTTGGGCAAAAGGTCGCTTTACCATTTCCAAAA | SWAKGRFTISKTSTTVDLRIT |
| | CATCCACTACTGTGGACCTCAGAATCACATCCCCAACTACAGAAGACACAGCCA | SPTTEDTATYFCAGGPTGNS |
| | CATACTTCTGCGCAGGAGGACCAACCGGCAACTCTCATTTCACACTCTGGGGC | HFTLWGQGTLVTVSSGGGG |
| | CAGGGCACTCTGGTCACCGTGTCATCTGGAGGTGGCGGATCAGGTGGCGGAG | SGGGGSGGGGSAIVMTQTP |
| | GTTCTGGCGGTGGCGGTTCTGCAATCGTGATGACCCAGACTCCTAGCTCCAAA | SSKSVPVGGTVTINCQASES |
| | AGCGTGCCCGTGGGTGGAACTGTTACGATCAACTGTCAGGCGAGCGAGTCCG | VYNNNHLSWYQQKSGQPPK |
| | TGTACAACAACAACCATCTTTCTTGGTATCAGCAGAAGTCTGGACAGCCGCCCA | LLIYEASKLESGVPPRFKGSG |
| | AGCTGCTCATATACGAAGCGTCAAAGCTGGAATCCGGAGTGCCACCTCGCTTT | SGTQFTLTISDWCDDAATYY |
| | AAGGGAAGCGGGAGTGGCACCCAGTTCACTCTGACCATAAGTGACGTTGTTTG | CSGYKRVTTDGIAFGGGTEV |
| | TGACGACGCTGCCACATATTATTGCAGTGGTTATAAGCGAGTCACCACGGACG | WKGSGEQKLISEEDL- |
| | GCATCGCCTTTGGAGGTGGCACCGAGGTGGTTGTGAAGGGCAGTGGAGAGCA | |
| | GAAGCTGATCTCAGAGGAGGACCTGtga | |
| MYCH15L- | atgCAGAGCCTGGAGGAATCAGGTGGAAGACTTGTTACACCAGGTACGCCCCTG | MQSLEESGGRLVTPGTPLTL |
| 28H3-28K1 | ACTCTGACATGTACTGTGAGCGGATTTTCCCTGAGTAGCTATAACATGGGATGG | TCTVSGFSLSSYNMGWVRQ |
| 236, 237 | GTCAGACAGGCCCCTGGTGAGGGTCTCGAGTGGATCGGTGTGATCGGTACCG | APGEGLEWIGVIGTGGITHYA |
| | GAGGTATCACTCACTATGCAACCTGGGCAAAAGGACGCGTGGCCATCAGCAGG | TWAKGRVAISRTSTTVGLRM |
| | ACCAGCACAACTGTAGGCCTGCGGATGACTAGCCCAACAACCGAAGACACAGC | TSPTTEDTATYFCVRSSGSD |
| | AACATACTTTTGTGTCCGGAGCAGCGGTTCCGATTGGTGGTTTCACATCTGGG | WWFHIWGQGTLVTVSSGGG |
| | GACAGGGAACTCTGGTCACCGTGTCATCTGGAGGTGGCGGATCAGGTGGCGG | GSGGGGSGGGGSAQVLTQT |
| | AGGTTCTGGCGGTGGCGGTTCTGCACAAGTGTTGACCCAGACTACTTCACCCG | TSPVSAAVGGTVTISCQSSQ |
| | TTAGTGCAGCCGTGGGAGGCACCGTTACCATTAGCTGCCAGTCATCCCAGTCA | SVYNNNNLAWFQQKPGQPP |
| | GTTTATAATAACAATAACCTTGCCTGGTTCCAGCAGAAACCAGGCCAGCCCCCC | KLLIYRASNLPSGVPSRFRGS |
| | AAGCTCCTGATCTATAGAGCCAGCAATCTGCCGAGCGGGGTTCCTTCCAGGTT | GSGSQFTLTISEVQCDDAAT |
| | CAGAGGGAGTGGTAGTGGGTCTCAGTTCACACTCACCATCAGCGAGGTGCAAT | YYCQGYFSGFITTFGGGTEV |
| | GTGATGACGCTGCTACCTACTACTGCCAGGGGTACTTCAGCGGATTCATCACA | WKGSGEQKLISEEDL- |
| | ACCTTTGGAGGTGGCACCGAGGTGGTTGTGAAGGGCAGTGGAGAGCAGAAGC | |
| | TGATCTCAGAGGAGGACCTGtga | |
| MYCH15L- | atgGCACAAGTGTTGACCCAGACTACTTCACCCGTTAGTGCAGCCGTGGGAGGC | MAQVLTQTTSPVSAAVGGTV |
| 14H1-14K2 | ACCGTTACCATTAGCTGCCAGTCATCCCAGTCAGTTTATAATAACAATAACCTTG | TISCQSSQSVYNNNNLAWFQ |
| 238, 239 | CCTGGTTCCAGCAGAAACCAGGCCAGCCCCCCAAGCTCCTGATCTATAGAGCC | QKPGQPPKLLIYRASNLPSG |
| | AGCAATCTGCCGAGCGGGGTTCCTTCCAGGTTCAGAGGGAGTGGTAGTGGGT | VPSRFRGSGSGSQFTLTISE |
| | CTCAGTTCACACTCACCATCAGCGAGGTGCAATGTGATGACGCTGCTACCTACT | VQCDDAATYYCQGYFSGFIT |
| | ACTGCCAGGGGTACTTCAGCGGATTCATCACAACCTTTGGAGGTGGCACCGAG | TFGGGTEVVVKGGGGSGGG |
| | GTGGTTGTGAAGGGAGGTGGCGGATCAGGTGGCGGAGGTTCTGGCGGTGGC | GSGGGGSQSLEESGGRLVT |
| | GGTTCTCAGAGCCTGGAGGAATCAGGTGGAAGACTTGTTACACCAGGTACGCC | PGTPLTLTCTVSGFSLSSYN |
| | CCTGACTCTGACATGTACTGTGAGCGGATTTTCCCTGAGTAGCTATAACATGGG | MGWRQAPGEGLEWIGVIG |
| | ATGGGTCAGACAGGCCCCTGGTGAGGGTCTCGAGTGGATCGGTGTGATCGGT | TGGITHYATWAKGRVAISRT |
| | ACCGGAGGTATCACTCACTATGCAACCTGGGCAAAAGGACGCGTGGCCATCAG | STTVGLRMTSPTTEDTATYF |
| | CAGGACCAGCACAACTGTAGGCCTGCGGATGACTAGCCCAACAACCGAAGACA | CVRSSGSDWWFHIWGQGTL |
| | CAGCAACATACTTTTGTGTCCGGAGCAGCGGTTCCGATTGGTGGTTTCACATCT | VTVSSGSGEQKLISEEDL- |
| | GGGGACAGGGAACTCTGGTCACCGTGTCATCTGGCAGTGGAGAGCAGAAGCT | |
| | GATCTCAGAGGAGGACCTGtga | |

e) MYC Constructs with a V_{L} Linked to a V_{H} Through a 20 Amino Acid Linker:

The VH and VL regions of each antibody were optimized and synthesized. Each scFv Fragment was amplified from the VH and VL templates and the 15aa linker was added at the same time. After PCR purification, the ScFv fragments were cloned into pcDNA3.1 vector by a seamless ligation protocol.

The MYC constructs with a V_{H} linked to a V_{L} through a 15 amino acid linker are MYCL20H-2F7, MYCL20H-1H3-1K3, MYCL20H-28H3-28K1, and MYCL20H-14H1-14K2. All of these constructs possess the general sequences of V_{L}—20 amino acid ScFv linker sequence—V_{H}—three amino acid MYC linker sequence—MYC Tag. The amino acid and polynucleotide sequences for constructs MYCL20H-2F7, MYCL20H-1H3-1K3, MYCL20H-28H3-28K1, and MYCL20H-14H1-14K2 are shown below in Table 13, The V_{H} and V_{L} regions for MYCL20H-2F7, MYCL20H-1H3-1K3, MYCL20H-28H3-28K1, and MYCL20H-14H1-14K2 are based on the corresponding V_{H} and V_{L} regions from antibodies 2F7, 1H3-1K3, 28H3-28K1, and 14H1-14K2 respectively.

TABLE 13
Polynucleotide and amino acid sequences for intrabodies for MYCL20H-2F7, MYCL20H-
1H3-1K3, MYCL20H-28H3-28K1, and MYCL20H-14H1-14K2. The MYC tag sequences are shown in
underlined font and the linker sequences is shown in bold font.

| Label | | |
|---|---|---|
| SEQ ID NO | Polynucleotide sequence | Amino acid sequence |
| MYCL20H- | atgGATGTGCTCATGACACAAACACCTTTGAGCCTTCCCGTGACACTCGGCGA | MDVLMTQTPLSLPVTLGDQAS |
| 2F7 | CCAGGCCAGTATCTCCTGCAGAAGTTCCCAGTCTATCGTGCACTCAAACGGT | ISCRSSQSIVHSNGNTYLEWY |
| 242, 243 | AATACGTACTTGGAGTGGTATCTGCAAAAACCCGGACAGTCCCCAAAGCTGT | LQKPGQSPKLLIYKVSNRFSG |
| | TGATATACAAGGTTTCTAATCGCTTCAGCGGTGTACCAGACAGATTTTCTGGC | VPDRFSGSGSGTDFTLKISRV |
| | TCCGGATCAGGAACCGACTTTACCCTTAAGATTTCAAGGGTGGAAGCCGAG | EAEDLGVYYCFQSSHVPWTG |
| | GACCTTGGAGTGTACTATTGCTTCCAATCAAGTCACGTCCCATGGACTGGGG | GGTKLEIKGSTGGGGSGKPG |
| | GCGGCACCAAGCTCGAGATCAAGGGCTCTACAGGAGGTGGCGGATCAGGT | SGEGGGGSDVKLVESGGGLV |
| | AAACCTGGTTCTGGCGAAGGAGGTGGCGGTTCAGATGTGAAGTTGGTTGAA | KLGGSLKLSCAASGFTFSSYY |
| | AGTGGAGGCGGTCTCGTTAAACTGGGCGGAAGTCTGAAACTTTCATGTGCA | MSWVRQTPEKRLELVATINSN |
| | GCTTCCGGGTTCACCTTCTCTAGCTATTACATGAGTTGGGTACGGCAGACCC | GGSTYYPDTVKGRITISRDNA |
| | CCGAAAAGCGACTGGAGCTGGTGGCTACCATAAATTCTAACGGAGGATCTAC | KNTLQLQMSSLRSEDTALYYC |
| | GTATTACCCCGACACCGTGAAGGGGCGCATCACCATTTCTCGGGACAATGC | VRQNYEGAYWGQGTLVTVSA |
| | CAAAAATACACTGCAGTTGCAGATGTCATCACTGCGAAGCGAAGACACCGCG | GSGEQKLISEEDL- |
| | CTGTACTACTGTGTAAGACAGAATTACGAGGGCGCATATTGGGGACAGGGC | |
| | ACTCTGGTCACCGTGTCAGCTGGCAGTGGAGAGCAGAAGCTGATCTCAGAG | |
| | GAGGACCTGtgat | |
| MYCL20H- | atgGCAATCGTGATGACCCAGACTCCTAGCTCCAAAAGCGTGCCCGTGGGTG | MAIVMTQTPSSKSVPVGGTVT |
| 1H3-1K3 | GAACTGTTACGATCAACTGTCAGGCGAGCGAGTCCGTGTACAACAACAACCA | INCQASESVYNNNHLSWYQQ |
| 244, 245 | TCTTTCTTGGTATCAGCAGAAGTCTGGACAGCCGCCCAAGCTGCTCATATAC | KSGQPPKLLIYEASKLESGVP |
| | GAAGCGTCAAAGCTGGAATCCGGAGTGCCACCTCGCTTTAAGGGAAGCGGG | PRFKGSGSGTQFTLTISDWC |
| | AGTGGCACCCAGTTCACTCTGACCATAAGTGACGTTGTTTGTGACGACGCTG | DDAATYYCSGYKRVTTDGIAF |
| | CCACATATTATTGCAGTGGTTATAAGCGAGTCACCACGGACGGCATCGCCTT | GGGTEVVVKGSTGGGGSGK |
| | TGGAGGTGGCACCGAGGTGGTTGTGAAGGGCTCTACAGGAGGTGGCGGAT | PGSGEGGGGSQSVEESGGR |
| | CAGGTAAACCTGGTTCTGGCGAAGGAGGTGGCGGTTCACAGAGCGTGGAG | LVTPGTPLTLTCTVSGFSLSRY |
| | GAATCAGGTGGAAGACTCGTTACTCCAGGTACTCCGCTGACCTTGACTTGTA | YMTWVRQAPGKGLEYIGVIIP |
| | CCGTGAGTGGCTTTTCCCTGTCCCGCTACTACATGACCTGGGTGAGGCAGG | GGTTYYASWAKGRFTISKTST |
| | CACCTGGCAAAGGGCTGGAATACATTGGGGTGATCATACCTGGCGGAACAA | TVDLRITSPTTEDTATYFCAGG |
| | CATATTACGCCAGTTGGGCAAAAGGTCGCTTTACCATTTCCAAAACATCCACT | PTGNSHFTLWGQGTLVTVSS |
| | ACTGTGGACCTCAGAATCACATCCCCAACTACAGAAGACACAGCCACATACT | GSGEQKLISEEDL- |
| | TCTGCGCAGGAGGACCAACCGGCAACTCTCATTTCACACTCTGGGGCCAGG | |
| | GCACTCTGGTCACCGTGTCATCTGGCAGTGGAGAGCAGAAGCTGATCTCAG | |
| | AGGAGGACCTGtga | |
| MYCL20H- | atgGCACAAGTGTTGACCCAGACTACTTCACCCGTTAGTGCAGCCGTGGGAG | MAQVLTQTTSPVSAAVGGTVT |
| 28H3-28K1 | GCACCGTTACCATTAGCTGCCAGTCATCCCAGTCAGTTTATAATAACAATAAC | ISCQSSQSVYNNNNLAWFQQ |
| 246, 247 | CTTGCCTGGTTCCAGCAGAAACCAGGCCAGCCCCCCAAGCTCCTGATCTATA | KPGQPPKLLIYRASNLPSGVP |
| | GAGCCAGCAATCTGCCGAGCGGGGTTCCTTCCAGGTTCAGAGGGAGTGGTA | SRFRGSGSGSQFTLTISEVQC |
| | GTGGGTCTCAGTTCACACTCACCATCAGCGAGGTGCAATGTGATGACGCTG | DDAATYYCQGYFSGFITTFGG |
| | CTACCTACTACTGCCAGGGGTACTTCAGCGGATTCATCACAACCTTTGGAGG | GTEVVVKGSTGGGGSGKPGS |
| | TGGCACCGAGGTGGTTGTGAAGGGCTCTACAGGAGGTGGCGGATCAGGTA | GEGGGGSQSLEESGGRLVTP |
| | AACCTGGTTCTGGCGAAGGAGGTGGCGGTTCACAGAGCCTGGAGGAATCA | GTPLTLTCTVSGFSLSSYNMG |
| | GGTGGAAGACTTGTTACACCAGGTACGCCCCTGACTCTGACATGTACTGTGA | WVRQAPGEGLEWIGVIGTGGI |
| | GCGGATTTTCCCTGAGTAGCTATAACATGGGATGGGTCAGACAGGCCCCTG | THYATWAKGRVAISRTSTTVG |
| | GTGAGGGTCTCGAGTGGATCGGTGTGATCGGTACCGGAGGTATCACTCACT | LRMTSPTTEDTATYFCVRSSG |
| | ATGCAACCTGGGCAAAAGGACGCGTGGCCATCAGCAGGACCAGCACAACTG | SDWWFHIWGQGTLVTVSSGS |
| | TAGGCCTGCGGATGACTAGCCCAACAACCGAAGACACAGCAACATACTTTTG | GEQKLISEEDL- |
| | TGTCCGGAGCAGCGGTTCCGATTGGTGGTTTCACATCTGGGGACAGGGAAC | |
| | TCTGGTCACCGTGTCATCTGGCAGTGGAGAGCAGAAGCTGATCTCAGAGGA | |
| | GGACCTGtga | |
| MYCL20H- | atgGCAATCGTGATGACCCAGACTCCTTCTAGTAAGAGTGTGCCTGTGGGAG | MAIVMTQTPSSKSVPVGGSVT |
| 14H1-14K2 | GGAGCGTGACAATCAACTGCCAGGCCAGCGAGTCCGTGTATAGTAATAATC | INCQASESVYSNNRLSWYQQ |
| 248, 249 | GACTCTCATGGTATCAACAGAAGCCCGGCCAGCCTCCTAAATTGTTGATATA | KPGQPPKLLIYYASTLESGVPS |
| | CTATGCAAGTACCCTCGAAAGCGGTGTTCCATCCAGGTTTAAAGGGAGCGG | RFKGSGFGTHFTLTISGAQCD |
| | CTTTGGGACTCACTTCACTCTTACGATTAGTGGAGCACAATGTGATGACGCA | DAATYYCAGWRGARTDGVDF |
| | GCTACTTATTATTGTGCCGGTTGGAGAGGGGCTAGAACCGATGGAGTGGATT | GGGTEVVVKGSTGGGGSGK |
| | TTGGAGGTGGCACCGAGGTGGTTGTGAAGGGCTCTACAGGAGGTGGCGGA | PGSGEGGGGSQEQLEESGG |
| | TCAGGTAAACCTGGTTCTGGCGAAGGAGGTGGCGGTTCACAGGAACAACT | DLVKPEGSLTLTCTASGFSFS |
| | GGAGGAATCAGGTGGAGATTTGGTGAAGCCGGAGGGGTCTCTGACCCTGAC | SNYVMCWVRQAPGKGLEWV |
| | ATGTACCGCCTCCGGGTTCAGTTTTTCCTCAAATTACGTCATGTGCTGGGTC | ACIWFAGIVDTTYYATWAKGR |
| | AGACAGGCGCCTGGAAAGGGGCTGGAATGGGTGGCATGCATTTGGTTTGCT | FTISKTSSTTVTLQMTSLTAAD |
| | GGAATTGTAGATACCACCTACTATGCCACGTGGGCGAAAGGACGGTTCACC | TATYFCARNPVGSVNLWGQG |
| | ATCAGCAAGACTAGTAGCACAACTGTTACCCTCCAGATGACAAGCCTGACCG | TLVTVSSGSGEQKLISEEDL- |
| | CAGCTGACACTGCCACTTATTTCTGCGCGCGCAACCCTGTTGGATCAGTTAA | |
| | TCTGTGGGGACAAGGCACTCTGGTCACCGTGTCATCTGGCAGTGGAGAGCA | |
| | GAAGCTGATCTCAGAGGAGGACCTGtga | |

f) MYC Constructs with a V_{H} Linked to a V_{L} Through a 20 Amino Acid Linker:

The VH and VL regions of each antibody were optimized and synthesized. Each scFv Fragment was amplified from the VH and VL templates and the 20aa linker was added at the same time. After PCR purification, the ScFv fragments were cloned into pcDNA3.1 vector by a seamless ligation protocol.

The MYC constructs with a V_{H} linked to a V_{L} through a 20 amino acid linker are, MYCH20L-2F7, MYCH20L-1H3-1K3, MYCH20L-28H3-28K1, and MYCH20L-14H1-14K2. All of these constructs possess the general sequences of V_{H}—20 amino acid ScFv linker sequence—V_{L}—three amino acid MYC linker sequence—MYC Tag. The amino acid and polynucleotide sequences for constructs, MYCH20L-2F7, MYCH20L-1H3-1K3, MYCH20L-28H3-28K1, and MYCH20L-14H1-14K2 are shown below in Table 14, The V_{H} and V_{L} regions for MYCH20L-2F7, MYCH20L-1H3-1K3, MYCH20L-28H3-28K1, and MYCH20L-14H1-14K2 are based on the corresponding V_{H} and V_{L} regions from antibodies 2F7, 1H3-1K3, 28H3-28K1, and 14H1-14K2 respectively.

TABLE 14
Polynucleotide and amino acid sequences for intrabodies MYCH20L-2F7, MYCH20L-1H3-
1K3, MYCH20L-28H3-28K1, and MYCH20L-14H1-14K2. The MYC tag sequences are shown in
underlined font and the linker sequences is shown in bold font.

| Label | | |
|---|---|---|
| (SEQ ID NO) | Polynucleotide sequence | Amino acid sequence |
| MYCH20L- | atgGATGTGAAGTTGGTTGAAAGTGGAGGCGGTCTCGTTAAACTGGGCGGAA | MDVKLVESGGGLVKLGGSLKL |
| 2F7 | GTCTGAAACTTTCATGTGCAGCTTCCGGGTTCACCTTCTCTAGCTATTACATG | SCAASGFTFSSYYMSWWRQT |
| 252, 253 | AGTTGGGTACGGCAGACCCCCGAAAAGCGACTGGAGCTGGTGGCTACCATA | PEKRLELVATINSNGGSTYYP |
| | AATTCTAACGGAGGATCTACGTATTACCCCGACACCGTGAAGGGGCGCATCA | DTVKGRITISRDNAKNTLQLQ |
| | CCATTTCTCGGGACAATGCCAAAAATACACTGCAGTTGCAGATGTCATCACT | MSSLRSEDTALYYCVRQNYE |
| | GCGAAGCGAAGACACCGCGCTGTACTACTGTGTAAGACAGAATTACGAGGG | GAYWGQGTLVTVSAGSTGGG |
| | CGCATATTGGGGACAGGGCACTCTGGTCACCGTGTCAGCTGGCTCTACAGG | GSGKPGSGEGGGGSDVLMT |
| | AGGTGGCGGATCAGGTAAACCTGGTTCTGGCGAAGGAGGTGGCGGTTCAG | QTPLSLPVTLGDQASISCRSS |
| | ATGTGCTCATGACACAAACACCTTTGAGCCTTCCCGTGACACTCGGCGACCA | QSIVHSNGNTYLEWYLQKPG |
| | GGCCAGTATCTCCTGCAGAAGTTCCCAGTCTATCGTGCACTCAAACGGTAAT | QSPKLLIYKVSNRFSGVPDRF |
| | ACGTACTTGGAGTGGTATCTGCAAAAACCCGGACAGTCCCCAAAGCTGTTGA | SGSGSGTDFTLKISRVEAEDL |
| | TATACAAGGTTTCTAATCGCTTCAGCGGTGTACCAGACAGATTTTCTGGCTCC | GVYYCFQSSHVPWTGGGTKL |
| | GGATCAGGAACCGACTTTACCCTTAAGATTTCAAGGGTGGAAGCCGAGGAC | EIKGSGEQKLISEEDL- |
| | CTTGGAGTGTACTATTGCTTCCAATCAAGTCACGTCCCATGGACTGGGGGCG | |
| | GCACCAAGCTCGAGATCAAGGGCAGTGGAGAGCAGAAGCTGATCTCAGAG | |
| | GAGGACCTGtga | |
| MYCH20L- | atgCAGAGCGTGGAGGAATCAGGTGGAAGACTCGTTACTCCAGGTACTCCGC | MQSVEESGGRLVTPGTPLTLT |
| 1H3-1K3 | TGACCTTGACTTGTACCGTGAGTGGCTTTTCCCTGTCCCGCTACTACATGAC | CTVSGFSLSRYYMTWVRQAP |
| 254, 255 | CTGGGTGAGGCAGGCACCTGGCAAAGGGCTGGAATACATTGGGGTGATCAT | GKGLEYIGVIIPGGTTYYASWA |
| | ACCTGGCGGAACAACATATTACGCCAGTTGGGCAAAAGGTCGCTTTACCATT | KGRFTISKTSTTVDLRITSPTT |
| | TCCAAAACATCCACTACTGTGGACCTCAGAATCACATCCCCAACTACAGAAG | EDTATYFCAGGPTGNSHFTL |
| | ACACAGCCACATACTTCTGCGCAGGAGGACCAACCGGCAACTCTCATTTCAC | WGQGTLVTVSSGSTGGGGS |
| | ACTCTGGGGCCAGGGCACTCTGGTCACCGTGTCATCTGGCTCTACAGGAGG | GKPGSGEGGGGSAIVMTQTP |
| | TGGCGGATCAGGTAAACCTGGTTCTGGCGAAGGAGGTGGCGGTTCAGCAA | SSKSVPVGGTVTINCQASESV |
| | TCGTGATGACCCAGACTCCTAGCTCCAAAAGCGTGCCCGTGGGTGGAACTG | YNNNHLSWYQQKSGQPPKLLI |
| | TTACGATCAACTGTCAGGCGAGCGAGTCCGTGTACAACAACAACCATCTTTC | YEASKLESGVPPRFKGSGSG |
| | TTGGTATCAGCAGAAGTCTGGACAGCCGCCCAAGCTGCTCATATACGAAGC | TQFTLTISDWCDDAATYYCS |
| | GTCAAAGCTGGAATCCGGAGTGCCACCTCGCTTTAAGGGAAGCGGGAGTGG | GYKRVTTDGIAFGGGTEVVVK |
| | CACCCAGTTCACTCTGACCATAAGTGACGTTGTTTGTGACGACGCTGCCACA | GSGEQKLISEEDL- |
| | TATTATTGCAGTGGTTATAAGCGAGTCACCACGGACGGCATCGCCTTTGGAG | |
| | GTGGCACCGAGGTGGTTGTGAAGGGCAGTGGAGAGCAGAAGCTGATCTCA | |
| | GAGGAGGACCTGtga | |
| MYCH20L- | atgCAGAGCCTGGAGGAATCAGGTGGAAGACTTGTTACACCAGGTACGCCCC | MQSLEESGGRLVTPGTPLTLT |
| 28H3-28K1 | TGACTCTGACATGTACTGTGAGCGGATTTTCCCTGAGTAGCTATAACATGGG | CTVSGFSLSSYNMGWVRQAP |
| 256, 257 | ATGGGTCAGACAGGCCCCTGGTGAGGGTCTCGAGTGGATCGGTGTGATCG | GEGLEWIGVIGTGGITHYATW |
| | GTACCGGAGGTATCACTCACTATGCAACCTGGGCAAAAGGACGCGTGGCCA | AKGRVAISRTSTTVGLRMTSP |
| | TCAGCAGGACCAGCACAACTGTAGGCCTGCGGATGACTAGCCCAACAACCG | TTEDTATYFCVRSSGSDWWF |
| | AAGACACAGCAACATACTTTTGTGTCCGGAGCAGCGGTTCCGATTGGTGGTT | HIWGQGTLVTVSSGSTGGGG |
| | TCACATCTGGGGACAGGGAACTCTGGTCACCGTGTCATCTGGCTCTACAGG | SGKPGSGEGGGGSAQVLTQT |
| | AGGTGGCGGATCAGGTAAACCTGGTTCTGGCGAAGGAGGTGGCGGTTCAG | TSPVSAAVGGTVTISCQSSQS |
| | CACAAGTGTTGACCCAGACTACTTCACCCGTTAGTGCAGCCGTGGGAGGCA | VYNNNNLAWFQQKPGQPPKL |
| | CCGTTACCATTAGCTGCCAGTCATCCCAGTCAGTTTATAATAACAATAACCTT | LIYRASNLPSGVPSRFRGSGS |
| | GCCTGGTTCCAGCAGAAACCAGGCCAGCCCCCCAAGCTCCTGATCTATAGA | GSQFTLTISEVQCDDAATYYC |
| | GCCAGCAATCTGCCGAGCGGGGTTCCTTCCAGGTTCAGAGGGAGTGGTAGT | QGYFSGFITTFGGGTEVVVKG |
| | GGGTCTCAGTTCACACTCACCATCAGCGAGGTGCAATGTGATGACGCTGCTA | SGEQKLISEEDL- |
| | CCTACTACTGCCAGGGGTACTTCAGCGGATTCATCACAACCTTTGGAGGTGG | |
| | CACCGAGGTGGTTGTGAAGGGCAGTGGAGAGCAGAAGCTGATCTCAGAGG | |
| | AGGACCTGtga | |
| MYCH20L- | atgCAGGAACAACTGGAGGAATCAGGTGGAGATTTGGTGAAGCCGGAGGGGT | MQEQLEESGGDLVKPEGSLT |
| 14H1-14K2 | CTCTGACCCTGACATGTACCGCCTCCGGGTTCAGTTTTTCCTCAAATTACGT | LTCTASGFSFSSNYVMCWVR |
| 258, 259 | CATGTGCTGGGTCAGACAGGCGCCTGGAAAGGGGCTGGAATGGGTGGCAT | QAPGKGLEWVACIWFAGIVDT |
| | GCATTTGGTTTGCTGGAATTGTAGATACCACCTACTATGCCACGTGGGCGAA | TYYATWAKGRFTISKTSSTTV |
| | AGGACGGTTCACCATCAGCAAGACTAGTAGCACAACTGTTACCCTCCAGATG | TLQMTSLTAADTATYFCARNP |
| | ACAAGCCTGACCGCAGCTGACACTGCCACTTATTTCTGCGCGCGCAACCCT | VGSVNLWGQGTLVTVSSGST |
| | GTTGGATCAGTTAATCTGTGGGGACAAGGCACTCTGGTCACCGTGTCATCTG | GGGGSGKPGSGEGGGGSAIV |
| | GCTCTACAGGAGGTGGCGGATCAGGTAAACCTGGTTCTGGCGAAGGAGGT | MTQTPSSKSVPVGGSVTINCQ |
| | GGCGGTTCAGCAATCGTGATGACCCAGACTCCTTCTAGTAAGAGTGTGCCT | ASESVYSNNRLSWYQQKPGQ |
| | GTGGGAGGGAGCGTGACAATCAACTGCCAGGCCAGCGAGTCCGTGTATAGT | PPKLLIYYASTLESGVPSRFKG |
| | AATAATCGACTCTCATGGTATCAACAGAAGCCCGGCCAGCCTCCTAAATTGT | SGFGTHFTLTISGAQCDDAAT |
| | TGATATACTATGCAAGTACCCTCGAAAGCGGTGTTCCATCCAGGTTTAAAGG | YYCAGWRGARTDGVDFGGG |
| | GAGCGGCTTTGGGACTCACTTCACTCTTACGATTAGTGGAGCACAATGTGAT | TEVVVKGSGEQKLISEEDL- |
| | GACGCAGCTACTTATTATTGTGCCGGTTGGAGAGGGGCTAGAACCGATGGA | |
| | GTGGATTTTGGAGGTGGCACCGAGGTGGTTGTGAAGGGCAGTGGAGAGCA | |
| | GAAGCTGATCTCAGAGGAGGACCTGtga | |

The scFv vector constructs were used for transfection and expression studies, as described in Example 13.

### Example 13

### Immunocytochemistry and Colocalization

The various constructs were expressed in HEK293 cells with or without dNLS-TDP43 using the methods described below. Immunocytochemistry and colocalization analysis was performed also as described below.

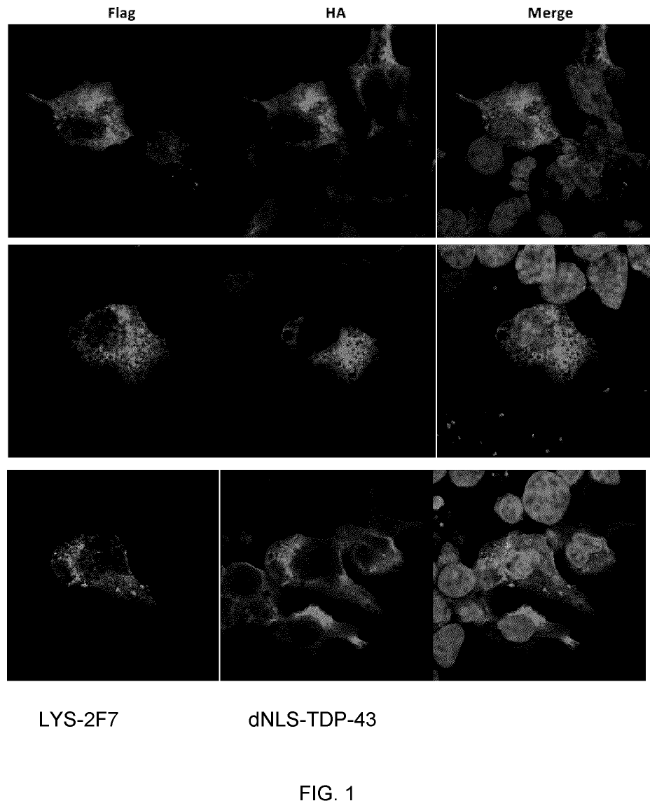

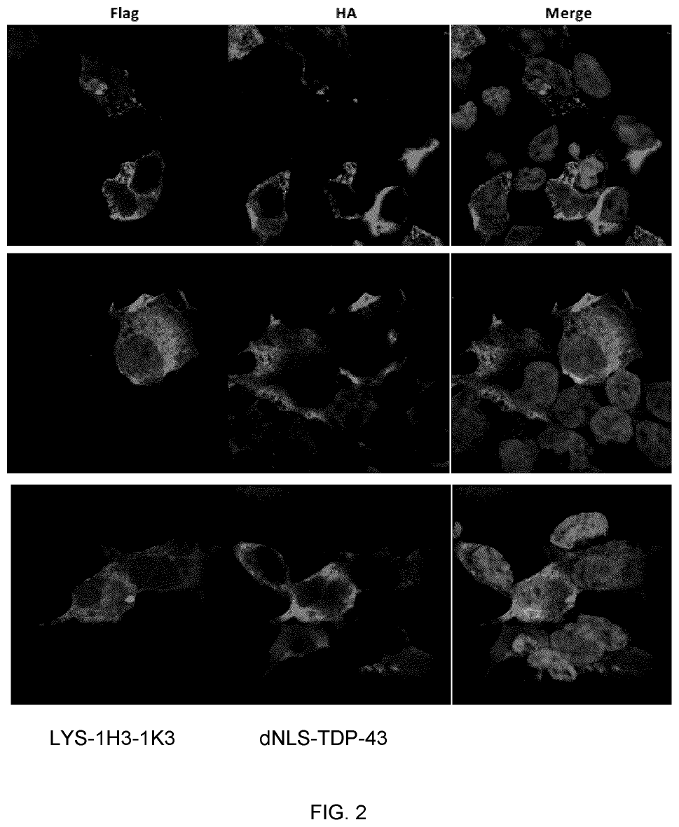

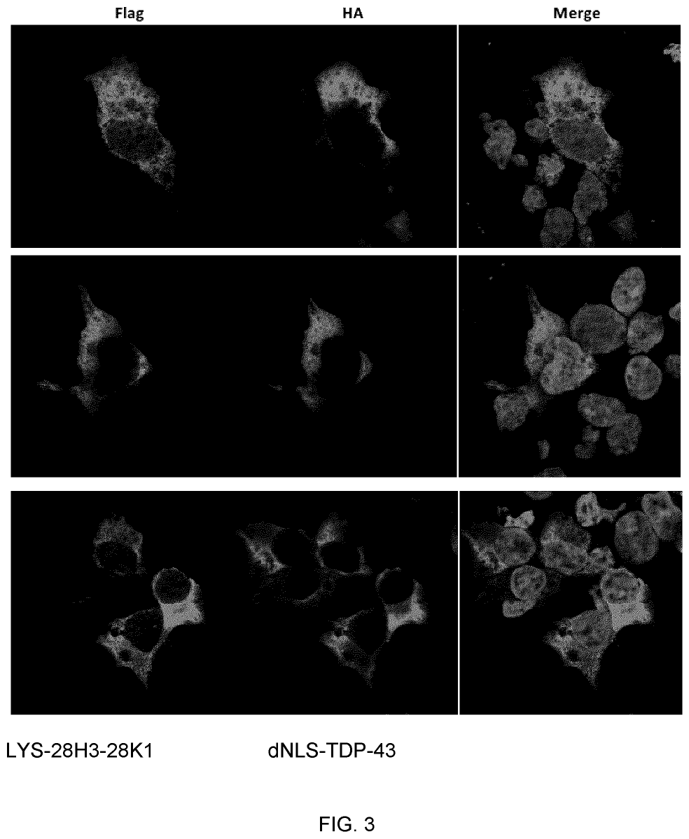

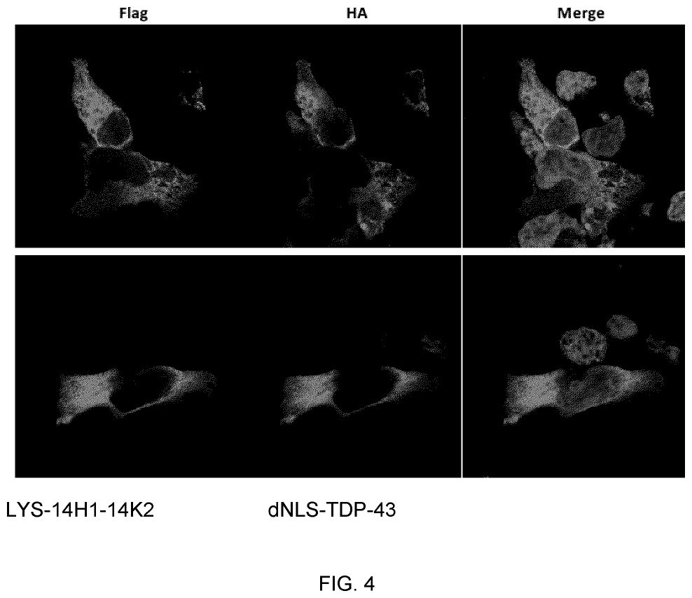

LYS-2F7, LYS-1H3-1K3, LYS-28H3-28K1, and LYS-14H1-14K2 TDP43 intrabodies interact with cytoplasmic aggregates of dNLS-TDP43 in transfected HEK293 cells (FIGS. 1-4). None of the intrabodies show appreciable interaction with endogenous wild type TDP43 in the nucleus. Overexpression of the TDP43 intrabodies does not affect cell viability indicating that they do not interfere with essential normal TDP43 function.

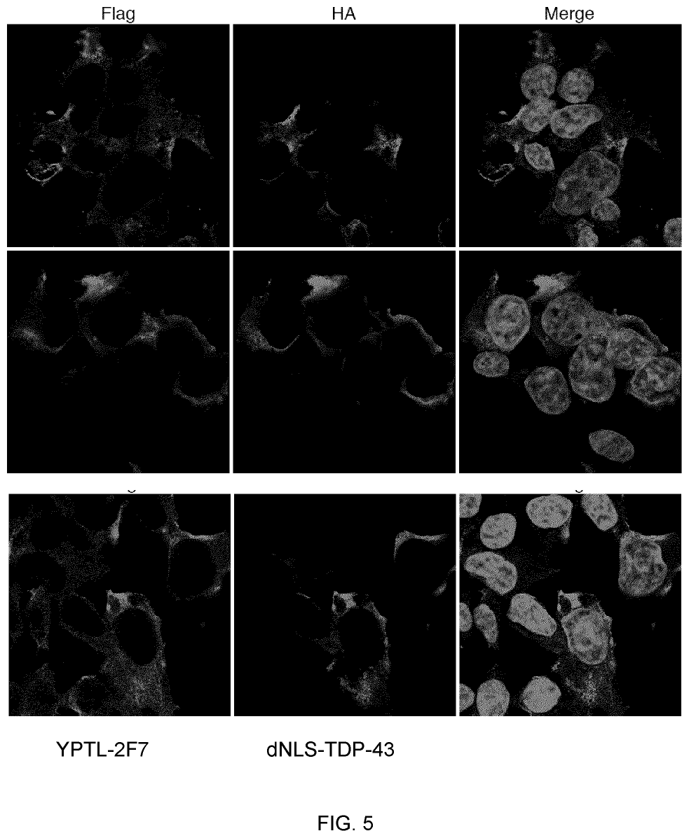

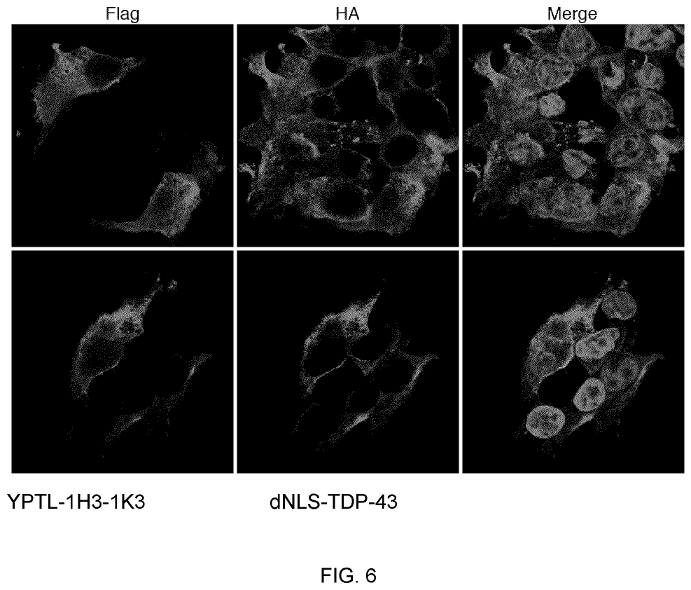

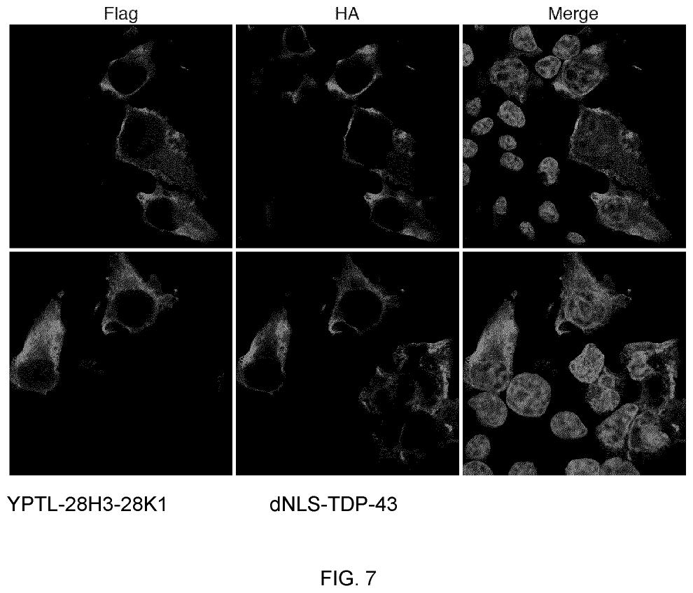

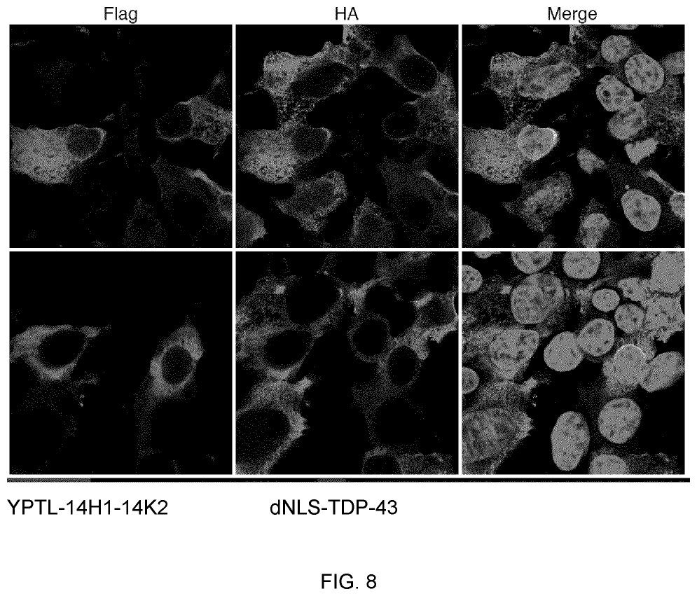

Similarly, overexpression of YPTL-2F7, YPTL-1H3-1K3, YPTL-28H3-28K1, and YPTL-14H1-14K2 TDP43 intrabodies does not affect the viability of HEK293 cells indicating that the intrabodies do not interfere with the essential functions of normal TDP43 (FIGS. 5-8). YPTL-2F7, YPTL-1H3-1K3, YPTL-28H3-28K1, and YPTL-14H1-14K2 all display strong interaction with aggregated cytoplasmic dNLS-TDP-43 but not normal nuclear TDP-43 (FIGS. 5-8).

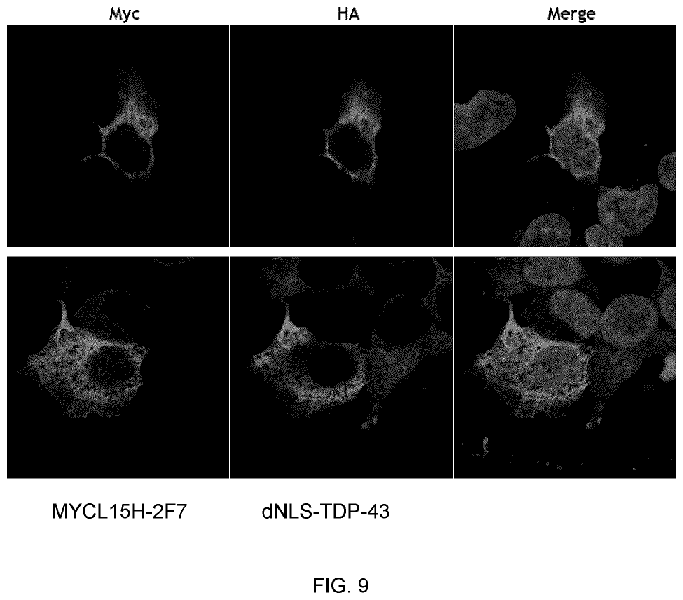

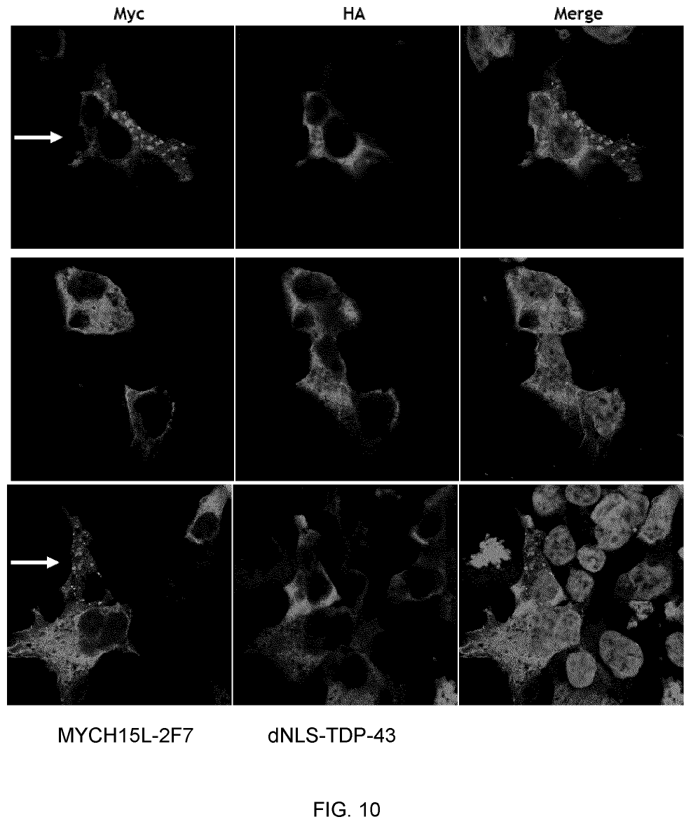

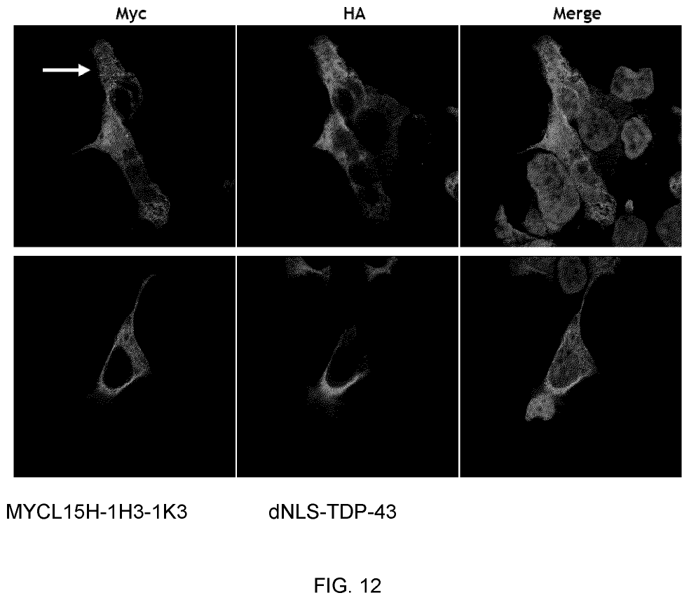

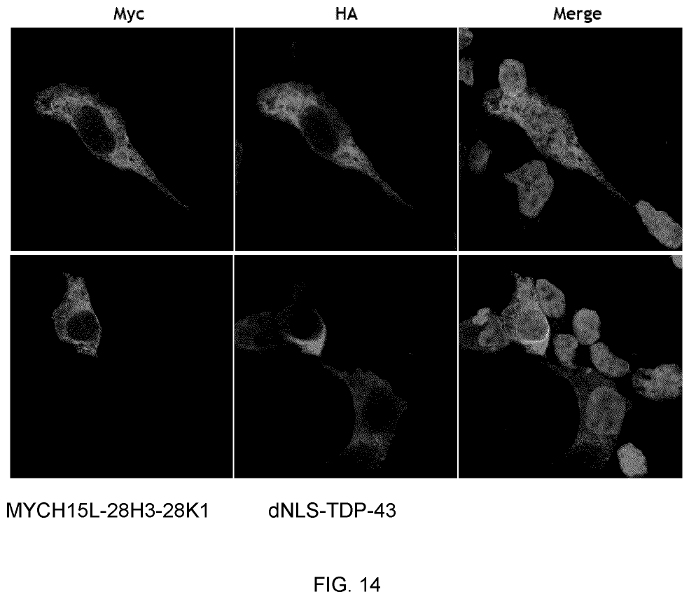

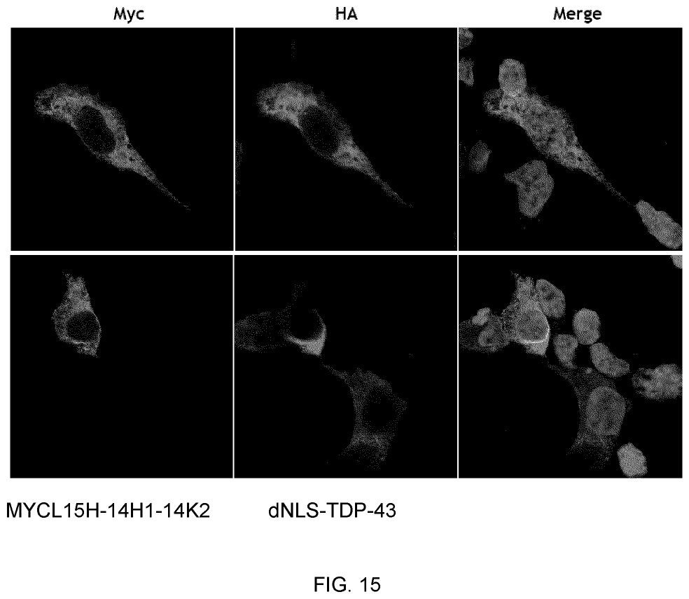

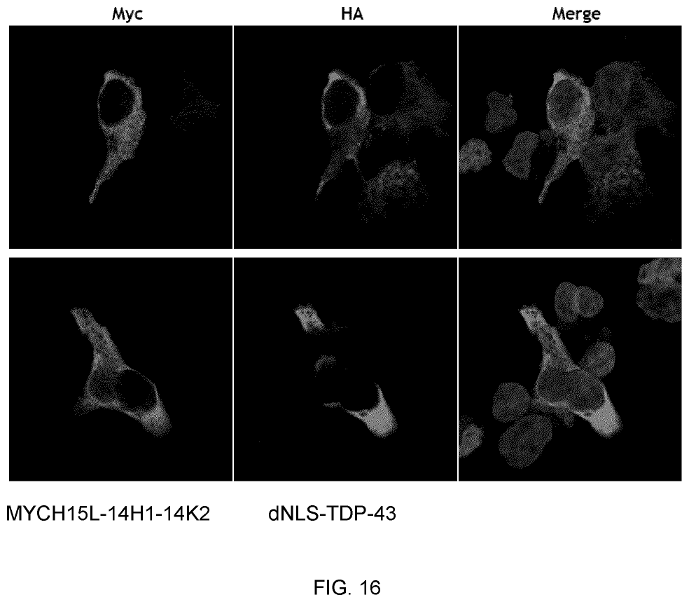

Overexpressed MYCL15H-2F7, MYCH15L-2F7, MYCL15H-1H3-1K3, MYCH15L-28H3-28K1, MYCL15H-14H1-14K2, and MYCH15L-14H1-14K2 TDP43 intrabodies colocalize with misfolded TDP43 cytoplasmic aggregates (FIGS. 9, 10, 12, 14, 15 and 16). None of the intrabodies show appreciable interaction with endogenous wild type TDP43 in the nucleus. MYCL15H-2F7, MYCH15L-2F7, MYCL15H-1H3-1K3, MYCH15L-28H3-28K1, MYCL15H-14H1-14K2, and MYCH15L-14H1-14K2 all display strong interaction with aggregated cytoplasmic dNLS-TDP-43 (FIGS. 9, 10, 12, 14, 15 and 16).

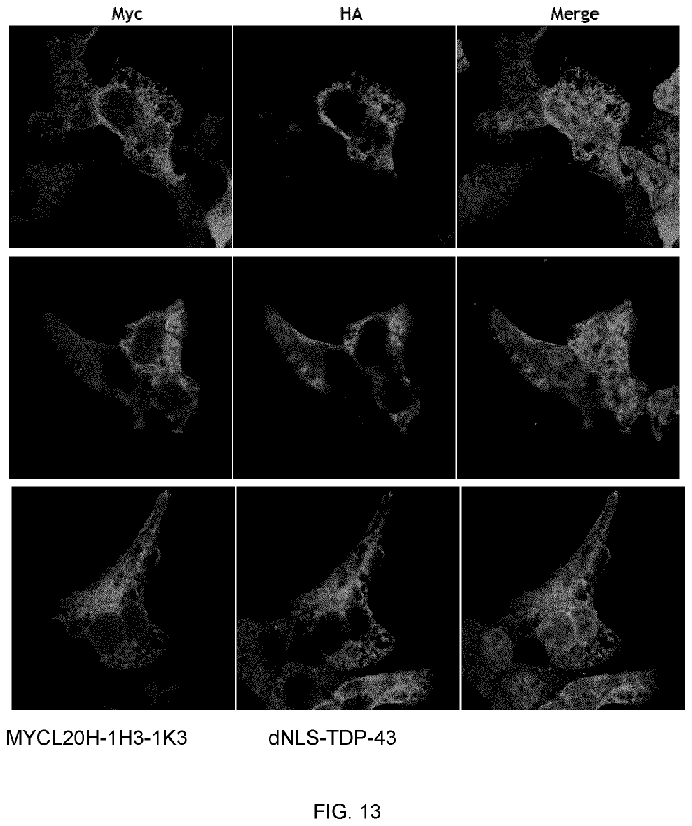

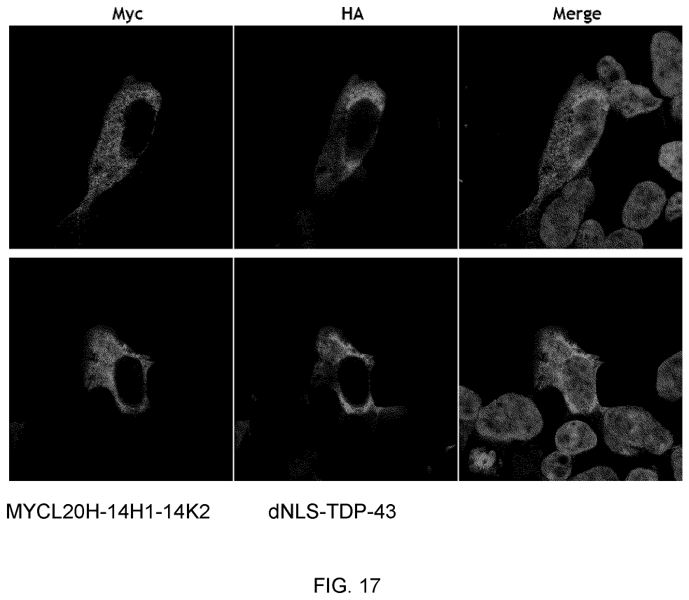

Overexpressed MYCL20H-1H3-1K3, and MYCL20H-14H1-14K2 TDP43 intrabodies colocalize with misfolded TDP43 cytoplasmic aggregates (FIGS. 13 and 17). MYCL20H-1H3-1K3, and MYCL20H-14H1-14K2 TDP43 display strong interaction with aggregated cytoplasmic dNLS-TDP-43 but not normal nuclear TDP-43 (FIGS. 13 and 17).

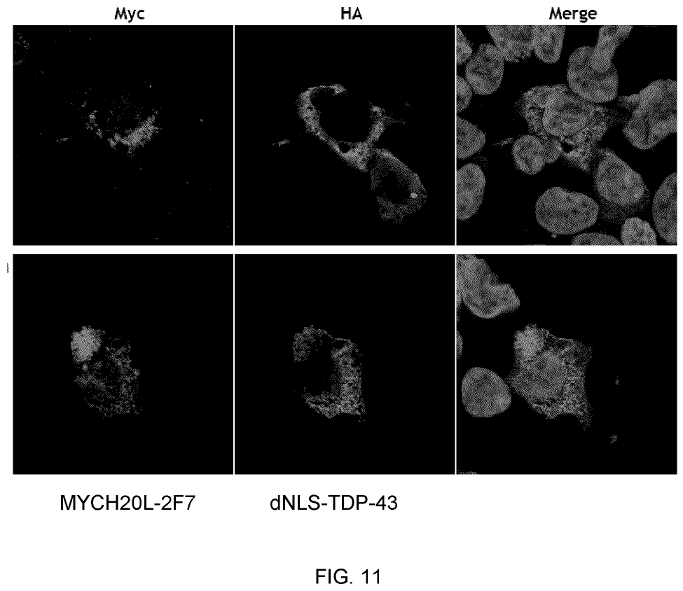

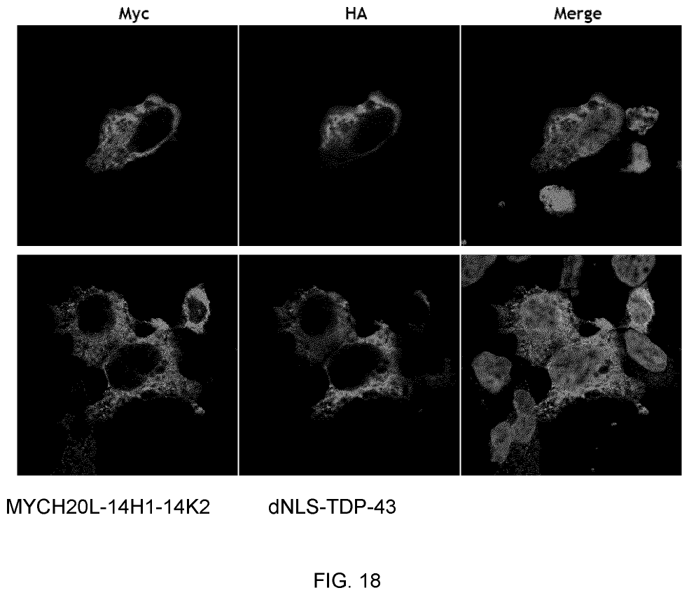

Overexpressed MYCH20L-14H1-14K2 TDP43 intrabody colocalizes with misfolded TDP43 cytoplasmic aggregates (FIG. 18). MYCH20L-2F7 showed lower expression (FIG. 11). None of the intrabodies show appreciable interaction with endogenous wild type TDP43 in the nucleus.

Degradation of dNLS-TDP-43

The effect of intrabody expression on dNLS-TDP-43 degradation was assessed as described below.

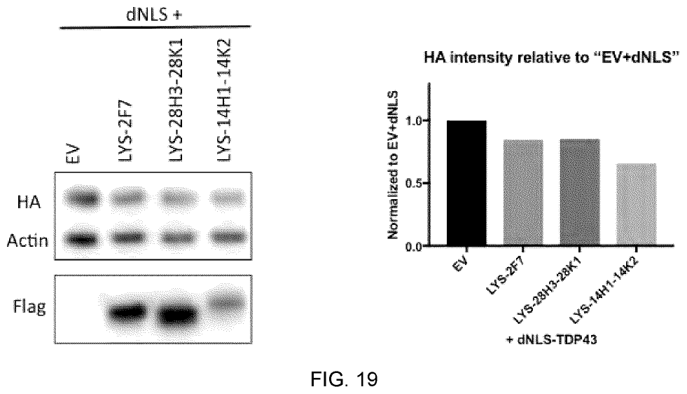

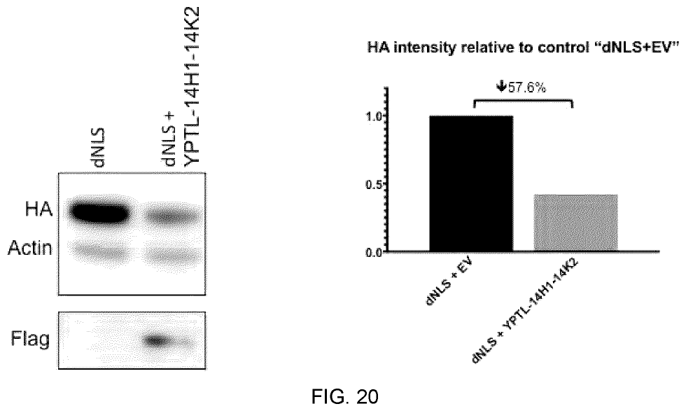

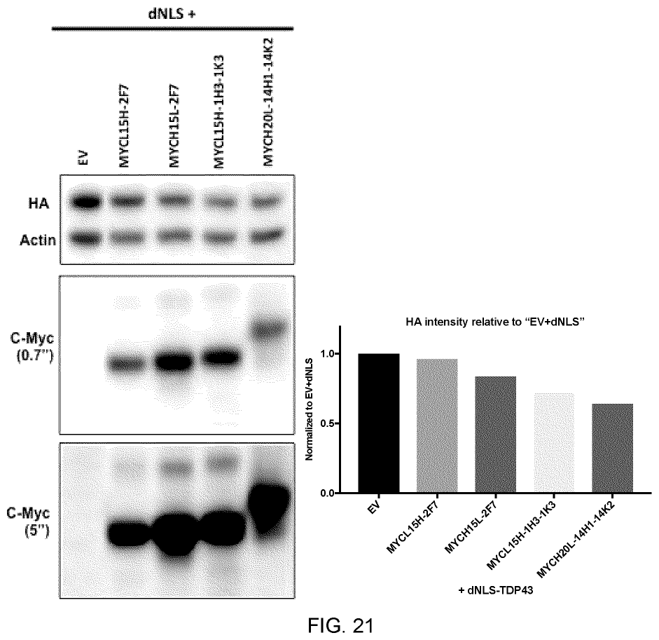

The results demonstrate LYS-2F7, LYS-28H3-28K1, and LYS-14H1-14K2, YPTL-14H1-14K2, MYCH15L-2F7, MYCL15H-1H3-1K3, and MYCH20L-14H1-14K2 intrabodies accelerate dNLS-TDP43 degradation (FIGS. 19, 20, and 21).

### Materials and Methods

Cell Culture and Transfection

Human embryonic kidney 293T (HEK293T) cell line was purchased from American Type Culture Collection (ATCC, Rockville, Md.), and maintained in Dulbecco's Modified Eagle Medium (DMEM) supplemented with 10% fetal bovine serum (FBS), GlutaMax™⁻1 (2 mM) and antibiotics (50 U/ml penicillin and 50 mg/ml streptomycin) at 37° C. in 5% CO₂.

HA-tagged dNLS-TDP43, a mutant of TDP43 lacking a functional nuclear localization signal (NLS), was co-transfected with each individual TDP43 single-chain variable fragment (scFv) plasmid or control empty vector (EV) at a ratio of 1:5 into HEK293T cells using Lipofectamine LTX reagent (ThermoFisher Scientific, Waltham, Mass., USA) following the manufacturer's instruction.

Immunocytochemistry for Detection of TDP43 Intrabodies with dNLS-TDP43

72 hrs post-transfection, cells were washed twice with Phosphate Saline Buffer (PBS) and fixed in 4% PFA for 15 min at room temperature (RT), followed by wash with 20 mM glycine for 10 min at RT with constant rocking to quench residual PFA. Cells were then incubated with blocking buffer containing PBS, 1% Bovine Serum Albumin (BSA), 10% normal goat serum, and 0.1% Triton-X-100 for 30 min at RT prior to addition of primary antibodies. To examine the interaction of the LYS and YPTL TDP43 intrabodies with dNLS-TDP43, mouse monoclonal anti-Flag (Sigma, St. Louis, Mich., USA, F1804, 1:2000) was used for detection of TDP43 scFv construct expression and rabbit polyclonal anti-HA (Abcam, Cambridge, UK, ab9110, 1:1000) for dNLS-TDP43. To examine the interaction of Myc TDP43 intrabodies with dNLS-TDP43, rabbit polyclonal anti-c-Myc (Abcam, ab9106, 1:1000) was used for detection of scFv expression and rat monoclonal anti-HA (Sigma, 1:2000) for dNLS-TDP43. After 2 hrs of primary antibody incubation, cells were washed with PBS/0.1% Triton-X-100 (PBST) 3×10 min with constant rocking, followed by incubation with Alexa Fluor® goat anti-rabbit, -mouse, or -rat secondary antibody (ThermoFisher Scientific, 1:1000) for 30 min at RT in the dark. Cells were then washed with PBST 3×10 min, dipped in 5% PBS, and mounted with ProLong Gold Anti-fade Reagent with DAPI (ThermoFisher, P36931). Cells were analyzed by confocal microscopy (Leica TCS SP8 MP, Wetzlar, Germany).

### Western Blotting and Degradation Quantification

72 hrs post-transfection, cells were washed twice with cold PBS and lysed in 2% SDS, followed by sonication at 30% power for 15 sec to extract total protein. Protein content was determined by BCA assay (ThermoFisher Scientific). 10 pg of protein was separated on 4-12% NuPAGE Bis-Tris SDS-PAGE (ThermoFisher Scientific), transferred onto a PVDF membrane, and blocked in Tris buffered saline (TBS) containing 5% skim milk and 0.1% Tween-20 for 1 h at RT. The following primary antibodies were incubated overnight at 4 C: rabbit monoclonal anti-Flag (Cell Signaling Technology, Danvers, Mass., USA, 147935, 1:2000) for the LYS and YPTL constructs and rabbit anti-c-Myc (Abcam, 1:500) for MYC tagged constructs TDP43 scFv expression detection, rabbit anti-HA (Abcam, 1:4000) for dNLS-TDP43 detection, and mouse anti-β-Actin (abm, Richmond, BC, Canada, G043, 1:1000) for loading control. Membranes were then washed with TBS/0.1% Tween (TBST) 3×10 min at RT with constant rocking, followed by horseradish peroxidase (HRP)-conjugated goat anti-mouse (Sigma, 1:5000) or donkey anti-rabbit secondary antibody (Sigma, AP182P, 1:5000) incubation for 30 min at RT. Membranes were then washed with TBST 3×10 min, and developed with SuperSignal™ West Femto Maximum Sensitivity Substrate (ThermoFisher Scientific). Images were captured on a ChemiDoc™ MP Imager (Bio-Rad Laboratories, Hercules, Calif., USA).

ImageJ was used to measure the intensity of Western blot bands. To quantitate the effect of TDP43 scFv intrabodies on dNLS-TDP43 degradation, HA intensity was first normalized by corresponding β-actin for each transfection condition. Normalized HA intensity of each dNLS-TDP43/scFv co-transfection was then compared with “dNLS+EV” control.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

[1] Kuo P H, Chiang C H, Wnag Y T, Doudeva L G, Yuan H S, The Crystal Structure of TDP-43 RRM1-DNA Complex Reveals the Specific Recognition for UG- and TG-Rich Nucleic Acids. Nucleic Acids Res., 2014, vol 42, 4712.
[4] Arai, T., Hasegawa, M., Akiyama, H., Ikeda, K., Nonaka, T., Mori, H., Mann, D., Tsuchiya, K., Yoshida, M., Hashizume, Y., and Oda, T. TDP-43 is a component of ubiquitin-positive tau-negative inclusions in frontotemporal lobar degeneration and amyotrophic lateral sclerosis. Biochem. Biophys. Res. Commun., 2006, 351, 602-611.
[5] Chantelle F. Sephton, Shannon K. Good, Stan Atkin, Colleen M. Dewey, Paul Mayer III, Joachim Herz, and Gang Yu J. Biol. Chem. 2010, vol. 285, No. 9, 6826-6834.
[6] Abel, O., Powell, J. F., Andersen, P. M., and Al-Chalabi, A. Hum Mutat, 2012, 33:1345-51.
[7] Hamley, I. W. PEG-Peptide Conjugates 2014; 15, 1543-1559; dx.doi.org/10.1021/bm500246w.
[8] Roberts, M J et al Chemistry for peptide and protein PEGylation 64: 116-127.
[9] Afroz, T et al. Functional and dynamic polymerization of the ALS-linked protein TDP-43 antagonizes its pathologic aggregation. Nat Comm (2017) 8:45.
[10] Porta, S et al. Patient-derived frontotemporal lobar degeneration brain extracts induce formation and spreading of TDP-43 pathology in vivo. Nature Comm (2018) 9:4220.
[11] Brettschneider, J et al. Sequential distribution of pTDP-43 pathology in behavioral variant frontotemporal dementia (bvFTD). Acta Neuropathol (2014) 127: 423.
[12] Feiler, M. S. et al. TDP-43 is intracellularly transmitted across axon terminals. J Cell Biol (2015) 211: 897.

1. A nucleic acid comprising a sequence encoding a single chain antibody, the single chain antibody binding Trp-68 in misfolded TDP-43 and comprising a heavy chain variable region comprising complementarity determining regions CDR-H1, CDR-H2 and CDR-H3 and a light chain variable region comprising complementarity determining regions CDR-L1, CDR-L2 and CDR-L3, wherein the heavy chain variable region and the light chain variable region are linked by a linker and with the amino acid sequences of said CDRs comprising the sequences:
CDR-H1:
SEQ ID NO: 16
GFSFSSNYV;
CDR-H2:
SEQ ID NO: 17
IWFAGIVDTT;
CDR-H3:
SEQ ID NO: 18
ARNPVGSVNL;
CDR-L1:
SEQ ID NO: 19
ESVYSNNR;
CDR-L2:
SEQ ID NO: 20
YAS;
and
CDR-L3:
SEQ ID NO: 21
AGWRGARTDGVD,
CDR-H1:
SEQ ID NO: 10
GFSLSRYY;
CDR-H2:
SEQ ID NO: 11
HPGGTT;
CDR-H3:
SEQ ID NO: 12
AGGPTGNSHFTL;
CDR-L1:
SEQ ID NO: 13
ESVYNNNH;
CDR-L2:
SEQ ID NO: 14
EAS;
and
CDR-L3:
SEQ ID NO: 15
SGYKRVTTDGIA,
CDR-H1:
SEQ ID NO: 22
GFSFSSSYV;
CDR-H2:
SEQ ID NO: 23
SDTGINT;
CDR-H3:
SEQ ID NO: 24
ARRYTGDTYLGNFNL;
CDR-L1:
SEQ ID NO: 25
QSVYKNNY;
CDR-L2:
SEQ ID NO: 26
KAS;
and
CDR-L3:
SEQ ID NO: 27
AGGWRSLNA,
CDR-H1:
SEQ ID NO: 28
EFSFSSRYW;
CDR-H2:
SEQ ID NO: 29
IYTGSIDAT;
CDR-H3:
SEQ ID NO: 30
VRGSDAWGLYFNL;
CDR-L1:
SEQ ID NO: 31
QSIHKNNY;
CDR-L2:
SEQ ID NO: 32
FAS;
and
CDR-L3:
SEQ ID NO: 33
AGVYSGRIFA,
CDR-H1:
SEQ ID NO: 34
GFSLSSYT;
CDR-H2:
SEQ ID NO: 35
IYGGIGST;
CDR-H3:
SEQ ID NO: 36
GRGDI;
CDR-L1:
SEQ ID NO: 37
QSVYKNR;
CDR-L2:
SEQ ID NO: 38
GAS;
and
CDR-L3:
SEQ ID NO: 39
LGNYDCSSVDCGA,
CDR-H1:
SEQ ID NO: 40
GFSFSAYY;
CDR-H2:
SEQ ID NO: 41
TIPIGRT;
CDR-H3:
SEQ ID NO: 42
AGGPTGNSHFTL;
CDR-L1:
SEQ ID NO: 43
ESVYNNNQ;
CDR-L2:
SEQ ID NO: 44
QAS;
and
CDR-L3:
SEQ ID NO: 45
AGYKSPTTDGIA,
CDR-H1:
SEQ ID NO: 46
GFSLSSYA;
CDR-H2:
SEQ ID NO: 47
IYNYET;
CDR-H3:
SEQ ID NO: 48
ARDIFRTNTNL;
CDR-L1:
SEQ ID NO: 49
QSVYKNNG;
CDR-L2:
SEQ ID NO: 50
FTS;
and
CDR-L3:
SEQ ID NO: 51
LGGYDCSSRVCGA,
CDR-H1:
SEQ ID NO: 120
GFSLSSYN;
CDR-H2:
SEQ ID NO: 121
IGTGGIT;
CDR-H3:
SEQ ID NO: 122
VRSSGSDWWFHI;
CDR-L1:
SEQ ID NO: 123
QSVYNNNN;
CDR-L2:
SEQ ID NO: 124
RAS;
and
CDR-L3:
SEQ ID NO: 125
QGYFSGFITT,
CDR-H1:
SEQ ID NO: 130
GFTFSSYY;
CDR-H2:
SEQ ID NO: 131
INSNGGST;
CDR-H3:
SEQ ID NO: 132
VRQNYEGAY;
CDR-L1:
SEQ ID NO: 133
QSIVHSNGNTY;
CDR-L2:
SEQ ID NO: 134
KVS;
and
CDR-L3:
SEQ ID NO: 135
FQSSHVPWT,
or
CDR-H1:
SEQ ID NO: 140
GFTFSSYY;
CDR-H2:
SEQ ID NO: 141
INTNGGST;
CDR-H3:
SEQ ID NO: 142
VRQNYEGAY;
CDR-L1:
SEQ ID NO: 143
QSIVHSNGNTY;
CDR-L2:
SEQ ID NO: 144
KVS;
and
CDR-L3:
SEQ ID NO: 145
FQSSHVPWT
preferably wherein the CDR sequences comprise SEQ ID Nos: 10-15, 16-21, 120-125 or 130-135.

2. The nucleic acid of claim 1, wherein the antibody comprises a heavy chain variable region comprising: i) an amino acid sequence as set forth in SEQ ID NO: 98, ii) an amino acid sequence at least 80%, at least 85%, at least 90% or at least 95% sequence identity to SEQ ID NO: 98, wherein the CDR sequences are as set forth in SEQ ID NOs: 10-12, or iii) a conservatively substituted amino acid sequence of i), and/or wherein the antibody comprises a light chain variable region comprising an amino acid sequence as set forth in SEQ ID NO: 99, ii) an amino acid sequence with at least 80%, at least 85%, at least 90% or at least 95% sequence identity to SEQ ID NO: 99, wherein the CDR sequences are as set forth in SEQ ID NOs: 13-15, or iii) a conservatively substituted amino acid sequence of i), optionally wherein the heavy chain variable region amino acid sequence is encoded by a nucleotide sequence as set out in SEQ ID NO: 76 or a codon degenerate or optimized version thereof and/or the light chain variable region amino acid sequence is encoded by a nucleotide sequence as set out in SEQ ID NO: 77 or a codon degenerate or optimized version thereof.

3. The nucleic acid of claim 1, wherein the antibody comprises a heavy chain variable region comprising: i) an amino acid sequence as set forth in SEQ ID NO: 100, ii) an amino acid sequence with at least 80%, at least 85%, at least 90% or at least 95% sequence identity to SEQ ID NO: 100, wherein the CDR sequences are as set forth in SEQ ID NOs: 16-18, or iii) a conservatively substituted amino acid sequence of i), and/or wherein the antibody comprises a light chain variable region comprising an amino acid sequence as set forth in SEQ ID NO: 101, ii) an amino acid sequence with at least 80%, at least 85%, at least 90% or at least 95% sequence identity to SEQ ID NO: 101, wherein the CDR sequences are as set forth in SEQ ID NOs: 19-21, or iii) a conservatively substituted amino acid sequence of i), optionally wherein the heavy chain variable region amino acid sequence is encoded by a nucleotide sequence as set out in SEQ ID NO: 78 or a codon degenerate or optimized version thereof and/or the light chain variable region amino acid sequence is encoded by a nucleotide sequence as set out in SEQ ID NO: 79 or a codon degenerate or optimized version thereof.

4. The nucleic acid of claim 1, wherein the antibody comprises a heavy chain variable region comprising: i) an amino acid sequence as set forth in SEQ ID NO: 102, ii) an amino acid sequence with at least 80%, at least 85%, at least 90% or at least 95% sequence identity to SEQ ID NO: 102, wherein the CDR sequences are as set forth in SEQ ID NOs: 22-24, or iii) a conservatively substituted amino acid sequence of i), and/or wherein the antibody comprises a light chain variable region comprising an amino acid sequence as set forth in SEQ ID NO: 103, ii) an amino acid sequence with at least 80%, at least 85%, at least 90% or at least 95% sequence identity to SEQ ID NO: 103, wherein the CDR sequences are as set forth in SEQ ID NOs: 25-27, or iii) a conservatively substituted amino acid sequence of i), optionally wherein the heavy chain variable region amino acid sequence is encoded by a nucleotide sequence as set out in SEQ ID NO: 80 or a codon degenerate or optimized version thereof and/or the light chain variable region amino acid sequence is encoded by a nucleotide sequence as set out in SEQ ID NO: 81 or a codon degenerate or optimized version thereof.

5. The nucleic acid of claim 1, wherein the antibody comprises a heavy chain variable region comprising: i) an amino acid sequence as set forth in SEQ ID NO: 104, ii) an amino acid sequence at least 80%, at least 85%, at least 90% or at least 95% sequence identity to SEQ ID NO: 104, wherein the CDR sequences are as set forth in SEQ ID NOs: 28-30, or iii) a conservatively substituted amino acid sequence of i), and/or wherein the antibody comprises a light chain variable region comprising an amino acid sequence as set forth in SEQ ID NO: 105, ii) an amino acid sequence with at least 80%, at least 85%, at least 90% or at least 95% sequence identity to SEQ ID NO: 105, wherein the CDR sequences are as set forth in SEQ ID NOs: 31-33, or iii) a conservatively substituted amino acid sequence of i), optionally wherein the heavy chain variable region amino acid sequence is encoded by a nucleotide sequence as set out in SEQ ID NO: 82 or a codon degenerate or optimized version thereof and/or the light chain variable region amino acid sequence is encoded by a nucleotide sequence as set out in SEQ ID NO: 83 or a codon degenerate or optimized version thereof.

6. The nucleic acid of claim 1, wherein the antibody comprises a heavy chain variable region comprising: i) an amino acid sequence as set forth in SEQ ID NO: 106, ii) an amino acid sequence with at least 80%, at least 85%, at least 90% or at least 95% sequence identity to SEQ ID NO: 106, wherein the CDR sequences are as set forth in SEQ ID NOs: 34-36, or iii) a conservatively substituted amino acid sequence of i), and/or wherein the antibody comprises a light chain variable region comprising an amino acid sequence as set forth in SEQ ID NO: 107, ii) an amino acid sequence with at least 80%, at least 85%, at least 90% or at least 95% sequence identity to SEQ ID NO: 107, wherein the CDR sequences are as set forth in SEQ ID NOs: 37-39, or iii) a conservatively substituted amino acid sequence of i), optionally wherein the heavy chain variable region amino acid sequence is encoded by a nucleotide sequence as set out in SEQ ID NO: 84 or a codon degenerate or optimized version thereof and/or the light chain variable region amino acid sequence is encoded by a nucleotide sequence as set out in SEQ ID NO: 85 or a codon degenerate or optimized version thereof.

7. The nucleic acid of claim 1, wherein the antibody comprises a heavy chain variable region comprising: i) an amino acid sequence as set forth in SEQ ID NO: 108, ii) an amino acid sequence at least 80%, at least 85%, at least 90% or at least 95% sequence identity to SEQ ID NO: 108, wherein the CDR sequences are as set forth in SEQ ID NOs: 40-42, or iii) a conservatively substituted amino acid sequence of i), and/or wherein the antibody comprises a light chain variable region comprising an amino acid sequence as set forth in SEQ ID NO: 109, ii) an amino acid sequence with at least 80%, at least 85%, at least 90% or at least 95% sequence identity to SEQ ID NO: 109, wherein the CDR sequences are as set forth in SEQ ID NOs: 43-45, or iii) a conservatively substituted amino acid sequence of i), optionally wherein the heavy chain variable region amino acid sequence is encoded by a nucleotide sequence as set out in SEQ ID NO: 86 or a codon degenerate or optimized version thereof and/or the light chain variable region amino acid sequence is encoded by a nucleotide sequence as set out in SEQ ID NO: 87 or a codon degenerate or optimized version thereof.

8. The nucleic acid of claim 1, wherein the antibody comprises a heavy chain variable region comprising: i) an amino acid sequence as set forth in SEQ ID NO: 110, ii) an amino acid sequence at least 80%, at least 85%, at least 90% or at least 95% sequence identity to SEQ ID NO: 110, wherein the CDR sequences are as set forth in SEQ ID NOs: 46-48, or iii) a conservatively substituted amino acid sequence of i), and/or wherein the antibody comprises a light chain variable region comprising an amino acid sequence as set forth in SEQ ID NO: 111, ii) an amino acid sequence with at least 80%, at least 85%, at least 90% or at least 95% sequence identity to SEQ ID NO: 111, wherein the CDR sequences are as set forth in SEQ ID NOs: 49-51, or iii) a conservatively substituted amino acid sequence of i), optionally wherein the heavy chain variable region amino acid sequence is encoded by a nucleotide sequence as set out in SEQ ID NO: 88 or a codon degenerate or optimized version thereof and/or the light chain variable region amino acid sequence is encoded by a nucleotide sequence as set out in SEQ ID NO: 89 or a codon degenerate or optimized version thereof.

9. The nucleic acid of claim 1, wherein the antibody comprises a heavy chain variable region comprising: i) an amino acid sequence as set forth in SEQ ID NO: 128, ii) an amino acid sequence with at least 80%, at least 85%, at least 90% or at least 95% sequence identity to SEQ ID NO: 128, wherein the CDR sequences are as set forth in SEQ ID NOs: 120-122, or iii) a conservatively substituted amino acid sequence of i), and/or wherein the antibody comprises a light chain variable region comprising an amino acid sequence as set forth in SEQ ID NO: 129, ii) an amino acid sequence with at least 80%, at least 85%, at least 90% or at least 95% sequence identity to SEQ ID NO: 129, wherein the CDR sequences are as set forth in SEQ ID NOs: 123-125, or iii) a conservatively substituted amino acid sequence of i), optionally wherein the heavy chain variable region amino acid sequence is encoded by a nucleotide sequence as set out in SEQ ID NO: 126 or a codon degenerate or optimized version thereof and/or the light chain variable region amino acid sequence is encoded by a nucleotide sequence as set out in SEQ ID NO: 127 or a codon degenerate or optimized version thereof.

10. The nucleic acid of claim 1, wherein the antibody comprises a heavy chain variable region comprising: i) an amino acid sequence as set forth in SEQ ID NO: 138, ii) an amino acid sequence at least 80%, at least 85%, at least 90% or at least 95% sequence identity to SEQ ID NO: 138, wherein the CDR sequences are as set forth in SEQ ID NOs: 130-132, or iii) a conservatively substituted amino acid sequence of i), and/or wherein the antibody comprises a light chain variable region comprising an amino acid sequence as set forth in SEQ ID NO: 139, ii) an amino acid sequence with at least 80%, at least 85%, at least 90% or at least 95% sequence identity to SEQ ID NO: 139, wherein the CDR sequences are as set forth in SEQ ID NOs: 133-135, or iii) a conservatively substituted amino acid sequence of i), optionally wherein the heavy chain variable region amino acid sequence is encoded by a nucleotide sequence as set out in SEQ ID NO: 136 or a codon degenerate or optimized version thereof and/or the light chain variable region amino acid sequence is encoded by a nucleotide sequence as set out in SEQ ID NO: 137 or a codon degenerate or optimized version thereof.

11. The nucleic acid of claim 1, wherein the antibody comprises a heavy chain variable region comprising: i) an amino acid sequence as set forth in SEQ ID NO: 148, ii) an amino acid sequence at least 80%, at least 85%, at least 90% or at least 95% sequence identity to SEQ ID NO: 148, wherein the CDR sequences are as set forth in SEQ ID NOs: 140-142, or iii) a conservatively substituted amino acid sequence of i), and/or wherein the antibody comprises a light chain variable region comprising an amino acid sequence as set forth in SEQ ID NO: 149, ii) an amino acid sequence with at least 80%, at least 85%, at least 90% or at least 95% sequence identity to SEQ ID NO: 149, wherein the CDR sequences are as set forth in SEQ ID NOs: 143-145, or iii) a conservatively substituted amino acid sequence of i), optionally wherein the heavy chain variable region amino acid sequence is encoded by a nucleotide sequence as set out in SEQ ID NO: 146 or a codon degenerate or optimized version thereof and/or the light chain variable region amino acid sequence is encoded by a nucleotide sequence as set out in SEQ ID NO: 147 or a codon degenerate or optimized version thereof.

12. The nucleic acid of any one of claims 1 to 11, wherein the orientation of the heavy chain variable domain, linker and the light chain variable domain is heavy chain variable domain—linker—light chain variable domain.

13. The nucleic acid of any one of claims 1 to 11, wherein the orientation of the heavy chain variable domain, linker and the light chain variable domain is light chain variable domain—linker—heavy chain variable domain.

14. The nucleic acid of any one of claims 1 to 13, wherein the linker about 10 to about 25 amino acids, preferably 15 or 20 amino acids.

15. The nucleic acid of any one of claims t to 14, wherein the linker is selected from (Gly-Gly-Gly-Gly-Ser)₃ (SEQ ID NO: 180), (Gly-Gly-Gly-Gly-Ser)₄ (SEQ ID NO: 181), and GSTGGGGSGKPGSGEGGGGS (SEQ ID NO: 182).

16. The nucleic acid of any one of claims 1 to 15, wherein the nucleic acid further encodes a targeting moiety, optionally linked by a targeting moiety linker

17. The nucleic acid of claim 16, wherein the targeting moiety is selected from a lysosomal targeting sequence and an autophagy targeting sequence.

18. The nucleic acid of claim 17, wherein the lysosomal targeting sequence has an amino acid sequence selected from YPTL (SEQ ID NO: 173), KSIRSGYEVM (SEQ ID NO: 174) and RWRKSHSSSYTPLSGSTYPEGRH (SEQ ID NO: 175).

19. The nucleic acid of any one of claims 1 to 18, wherein the single chain antibody is an intrabody.

20. The nucleic acid of any one of claims 1 to 18, wherein the nucleic acid further encodes a signal peptide and/or a cell penetrating peptide.

21. The nucleic acid of any one of claims 1 to 20, wherein the single chain antibody is a scFV, minibody or nanobody.

22. The nucleic acid of any one of claims 1 to 21, wherein the single chain antibody is humanized.

23. The nucleic acid of any one of claims 1 to 11, wherein the sequence encodes the antibody and linker portions and optionally lysosmal targeting sequences of any of SEQ ID NOs: 203, 205, 207, 209, 213, 215, 217, 219, 223, 225, 227, 229, 233, 235, 237, 239, 243, 245, 247, 249, 253, 255, 257 and/or 259.

24. The nucleic acid of any one of claims 1 to 11, wherein the sequence comprises any one of SEQ ID NOs: 76-89, SEQ ID NOs: 126-127, SEQ ID NOs: 136-137, 146-147 or a codon optimized sequence thereof.

25. The nucleic acid of any one of claims 1 to 11, wherein the sequence comprises the antibody and linker portions and optionally lysosomal targeting sequences of any of SEQ ID NOs: 202, 204, 206, 208, 212, 214, 216, 218, 222, 224, 226, 228, 232, 234, 236, 238, 242, 244, 246, 248, 252, 254, 256 and/or 258.

26. An expression cassette or vector comprising the nucleic acid of any one of claims 1 to 25.

27. The vector of claim 26, wherein the vector is a lentiviral vector or adeno associated virus (AAV) vector, optionally AAV serotype 9.

28. A single chain antibody encoded by the nucleic acid of any one of claims 1 to 25, the expression cassette or vector of claim 26 or 27.

29. An immunoconjugate comprising the antibody of claim 28 and a detectable label.

30. A cell recombinantly expressing the single chain antibody encoded by the nucleic acid of any one of claims 1 to 25, or the vector of claim 26 or 27.

31. A composition comprising, the nucleic acid of any one of claims 1 to 25, the expression cassette or vector of claim 26 or 27, the single chain antibody of claim 28, the immunoconjugate of claim 29 or the cell of claim 30, optionally with a diluent, and/or a pharmaceutically acceptable carrier.

32. The composition of claim 31 comprising two or more different nucleic acids, optionally with a diluent, and/or a pharmaceutically acceptable carrier.

33. The composition of claim 31 comprising two or more different expression cassettes or vectors, optionally with a diluent, and/or a pharmaceutically acceptable carrier.

34. The composition of claim 31 comprising two or more different single chain antibodies, optionally with a diluent, and/or a pharmaceutically acceptable carrier.

35. The composition of claim 31 comprising two or more different immunoconjugates, optionally with a diluent, and/or a pharmaceutically acceptable carrier.

36. The composition of claim 31 comprising two or more different cells, optionally with a diluent, and/or a pharmaceutically acceptable carrier.

37. The composition of any one of claims 31 to 36, wherein the composition comprises lipid particles, optionally liposomes, nanoparticles or nanosomes.

38. The composition of any one of claims 31 to 37, formulated suitably for intrathecal, intraparenchymal or intraventricular administration.

39. A method of treating a subject, the method comprising administering to the subject in need thereof an effective amount of the nucleic acid of any one of claims 1 to 25, the expression cassette or vector of claim 26 or 27, the antibody of claim 28, or the composition of any one of claims 31 to 38, optionally one ore more nucleic acids, one or more expression cassettes or vectors, or one or more antibodies and/or compositions.

40. The method of claim 39, wherein the method comprises administering to the subject in need thereof an effective amount of two or more nucleic acids of any one of claims 1 to 25.

41. The method of claim 39, wherein the method comprises administering to the subject in need thereof an effective amount of two or more expression cassettes or vectors of claim 26 or 27.

42. The method of claim 39, wherein, the method comprises administering to the subject in need thereof an effective amount of two or more antibodies of claim 28.

43. The method of claim 39, wherein the method comprises to a subject in need thereof an effective amount of two or more compositions of any one of claims 31 to 38.

44. The method of any one of claims 39 to 43, wherein the subject is suspected of having, is at risk of developing, or has been diagnosed with a TDP-43 proteinopathy.

45. The method of claim 44, wherein the TDP-43 proteinopathy is selected from amyotrophic lateral sclerosis (ALS), frontotemporal lobar degeneration (FTLD-TDP), primary lateral sclerosis, progressive muscular atrophy, and limbic-predominant age-related TDP-43 encephalopathy (LATE).

46. The method of any one of claims 39 to 45, wherein the nucleic acid, expression cassette or vector, antibody, or composition is administered in combination with another TDP-43 proteinopathy treatment.

47. The method of any one of claims 39 to 46, wherein the nucleic acid, expression cassette, vector, single chain antibody, or composition is administered by parenteral, intravenous, subcutaneous, intramuscular, intracranial, intraventricular, intrathecal, intraorbital, ophthalmic, intraspinal, intracisternal, intraperitoneal, intranasal, aerosol or oral administration.


SEQUENCE LISTING (259 sequences)

SEQ ID NO: 1 - protein, 7 aa, Homo Sapiens
DAGWGNL

SEQ ID NO: 2 - protein, 5 aa, Homo Sapiens
DAGWG

SEQ ID NO: 3 - protein, 6 aa, Homo Sapiens
DAGWGN

SEQ ID NO: 4 - protein, 5 aa, Homo Sapiens
AGWGN

SEQ ID NO: 5 - protein, 6 aa, Homo Sapiens
AGWGNL

SEQ ID NO: 6 - protein, 5 aa, Homo Sapiens
GWGNL

SEQ ID NO: 7 - protein, 21 aa, Homo Sapiens
EGILHAPDAGWGNLVYVVNYP

SEQ ID NO: 8 - protein, 8 aa, Artificial Sequence
  Synthetic Construct
CDAGWGNL

SEQ ID NO: 9 - protein, 8 aa, Artificial Sequence
  Synthetic Construct
DAGWGNLC

SEQ ID NO: 10 - protein, 8 aa, Oryctolagus cuniculus
GFSLSRYY

SEQ ID NO: 11 - protein, 7 aa, Oryctolagus cuniculus
IIPGGTT

SEQ ID NO: 12 - protein, 12 aa, Oryctolagus cuniculus
AGGPTGNSHFTL

SEQ ID NO: 13 - protein, 8 aa, Oryctolagus cuniculus
ESVYNNNH

SEQ ID NO: 14 - protein, 3 aa, Oryctolagus cuniculus
EAS

SEQ ID NO: 15 - protein, 12 aa, Oryctolagus cuniculus
SGYKRVTTDGIA

SEQ ID NO: 16 - protein, 9 aa, Oryctolagus cuniculus
GFSFSSNYV

SEQ ID NO: 17 - protein, 10 aa, Oryctolagus cuniculus
IWFAGIVDTT

SEQ ID NO: 18 - protein, 10 aa, Oryctolagus cuniculus
ARNPVGSVNL

SEQ ID NO: 19 - protein, 8 aa, Oryctolagus cuniculus
ESVYSNNR

SEQ ID NO: 20 - protein, 3 aa, Oryctolagus cuniculus
YAS

SEQ ID NO: 21 - protein, 12 aa, Oryctolagus cuniculus
AGWRGARTDGVD

SEQ ID NO: 22 - protein, 9 aa, Oryctolagus cuniculus
GFSFSSSYV

SEQ ID NO: 23 - protein, 7 aa, Oryctolagus cuniculus
SDTGINT

SEQ ID NO: 24 - protein, 15 aa, Oryctolagus cuniculus
ARRYTGDTYLGNFNL

SEQ ID NO: 25 - protein, 8 aa, Oryctolagus cuniculus
QSVYKNNY

SEQ ID NO: 26 - protein, 3 aa, Oryctolagus cuniculus
KAS

SEQ ID NO: 27 - protein, 9 aa, Oryctolagus cuniculus
AGGWRSLNA

SEQ ID NO: 28 - protein, 9 aa, Oryctolagus cuniculus
EFSFSSRYW

SEQ ID NO: 29 - protein, 9 aa, Oryctolagus cuniculus
IYTGSIDAT

SEQ ID NO: 30 - protein, 13 aa, Oryctolagus cuniculus
VRGSDAWGLYFNL

SEQ ID NO: 31 - protein, 8 aa, Oryctolagus cuniculus
QSIHKNNY

SEQ ID NO: 32 - protein, 3 aa, Oryctolagus cuniculus
FAS

SEQ ID NO: 33 - protein, 10 aa, Oryctolagus cuniculus
AGVYSGRIFA

SEQ ID NO: 34 - protein, 8 aa, Oryctolagus cuniculus
GFSLSSYT

SEQ ID NO: 35 - protein, 8 aa, Oryctolagus cuniculus
IYGGIGST

SEQ ID NO: 36 - protein, 5 aa, Oryctolagus cuniculus
GRGDI

SEQ ID NO: 37 - protein, 7 aa, Oryctolagus cuniculus
QSVYKNR

SEQ ID NO: 38 - protein, 3 aa, Oryctolagus cuniculus
GAS

SEQ ID NO: 39 - protein, 13 aa, Oryctolagus cuniculus
LGNYDCSSVDCGA

SEQ ID NO: 40 - protein, 8 aa, Oryctolagus cuniculus
GFSFSAYY

SEQ ID NO: 41 - protein, 7 aa, Oryctolagus cuniculus
TIPIGRT

SEQ ID NO: 42 - protein, 12 aa, Oryctolagus cuniculus
AGGPTGNSHFTL

SEQ ID NO: 43 - protein, 8 aa, Oryctolagus cuniculus
ESVYNNNQ

SEQ ID NO: 44 - protein, 3 aa, Oryctolagus cuniculus
QAS

SEQ ID NO: 45 - protein, 12 aa, Oryctolagus cuniculus
AGYKSPTTDGIA

SEQ ID NO: 46 - protein, 8 aa, Oryctolagus cuniculus
GFSLSSYA

SEQ ID NO: 47 - protein, 6 aa, Oryctolagus cuniculus
IYNYET

SEQ ID NO: 48 - protein, 11 aa, Oryctolagus cuniculus
ARDIFRTNTNL

SEQ ID NO: 49 - protein, 8 aa, Oryctolagus cuniculus
QSVYKNNG

SEQ ID NO: 50 - protein, 3 aa, Oryctolagus cuniculus
FTS

SEQ ID NO: 51 - protein, 13 aa, Oryctolagus cuniculus
LGGYDCSSRVCGA

SEQ ID NO: 52 - protein, 8 aa, Oryctolagus cuniculus
GFTFSAYY

SEQ ID NO: 53 - protein, 7 aa, Oryctolagus cuniculus
TIPRGIT

SEQ ID NO: 54 - protein, 12 aa, Oryctolagus cuniculus
AGGPTGNSHFTL

SEQ ID NO: 55 - protein, 8 aa, Oryctolagus cuniculus
ESVYANNQ

SEQ ID NO: 56 - protein, 3 aa, Oryctolagus cuniculus
QAS

SEQ ID NO: 57 - protein, 12 aa, Oryctolagus cuniculus
SGYVRTTTDGIA

SEQ ID NO: 58 - protein, 8 aa, Oryctolagus cuniculus
GFSLIRYY

SEQ ID NO: 59 - protein, 7 aa, Oryctolagus cuniculus
TIPRGIT

SEQ ID NO: 60 - protein, 12 aa, Oryctolagus cuniculus
AGGPTGNSHFTL

SEQ ID NO: 61 - protein, 8 aa, Oryctolagus cuniculus
ESVYNNNQ

SEQ ID NO: 62 - protein, 3 aa, Oryctolagus cuniculus
QAS

SEQ ID NO: 63 - protein, 12 aa, Oryctolagus cuniculus
SGYKSTTTDGIA

SEQ ID NO: 64 - protein, 9 aa, Oryctolagus cuniculus
GFSFSSSYV

SEQ ID NO: 65 - protein, 9 aa, Oryctolagus cuniculus
IYFAGIDTI

SEQ ID NO: 66 - protein, 10 aa, Oryctolagus cuniculus
ARNPIGSVNL

SEQ ID NO: 67 - protein, 8 aa, Oryctolagus cuniculus
QSVYSNNR

SEQ ID NO: 68 - protein, 3 aa, Oryctolagus cuniculus
YLS

SEQ ID NO: 69 - protein, 12 aa, Oryctolagus cuniculus
AGWRGATTDGVD

SEQ ID NO: 70 - protein, 9 aa, Oryctolagus cuniculus
GFSFNSNYV

SEQ ID NO: 71 - protein, 10 aa, Oryctolagus cuniculus
IWFAGSGDVI

SEQ ID NO: 72 - protein, 10 aa, Oryctolagus cuniculus
ARNPVGSVNL

SEQ ID NO: 73 - protein, 8 aa, Oryctolagus cuniculus
ESVYINNR

SEQ ID NO: 74 - protein, 3 aa, Oryctolagus cuniculus
YAS

SEQ ID NO: 75 - protein, 12 aa, Oryctolagus cuniculus
AGWRGATTDGID

SEQ ID NO: 76 - DNA, 341 bp, Oryctolagus cuniculus
cagtcggtggaggagtccgggggtcgcctggtcacgcctgggacacccctgacactcacctgcacagtctctggattctccctcagtaggtactacatgacctgggtccgccaggctccagggaaggggctggaatacatcggggtcattattcctggtggtaccacatactacgcgagctgggcgaaaggccgattcaccatctccaaaacctcgaccacggtggatctgagaatcaccagtccgacaaccgaggacacggccacttatttctgtgccggaggtcctactggtaacagccactttacattgtggggccagggcaccctggtcaccgtctc

SEQ ID NO: 77 - DNA, 331 bp, Oryctolagus cuniculus
gtgatgacccagactccatcttccaagtctgtccctgtgggaggcacagtcaccatcaattgccaggccagtgagagtgtttataataacaaccacttatcctggtatcagcagaaatcagggcagcctcccaagctcctgatctacgaagcatccaaactggaatctggggtcccaccgcggttcaaaggcagtggatctgggacacagttcactctcaccatcagcgatgtggtgtgtgacgatgctgccacttactactgttcaggatataaacgtgttactactgatggtattgctttcggcggagggaccgaggtggtggtcaaag

SEQ ID NO: 78 - DNA, 353 bp, Oryctolagus cuniculus
caggagcagctggaggagtccgggggagacctggtcaagcctgagggatccctgacactcacctgcacagcctctggattctccttcagtagcaactacgtgatgtgctgggtccgccaggctccagggaaggggctggagtgggtcgcatgcatttggtttgctggtattgttgatactacttactacgcgacctgggcgaaaggccgattcaccatctccaaaacctcgtcgaccacggtgactctgcaaatgaccagtctgacagccgcggacacggccacctatttctgtgcgagaaatcctgttggtagtgtgaacttgtggggccagggcaccctggtcaccgtctc

SEQ ID NO: 79 - DNA, 331 bp, Oryctolagus cuniculus
gtgatgacccagactccatcttccaagtctgtccctgtgggaggctcagtcaccatcaattgccaggccagtgagagtgtttatagtaacaaccgcttatcctggtatcagcagaaaccagggcagcctcctaagctcctgatctattatgcatccactctggaatctggggtcccatcgcggttcaaaggcagtggatttgggacacacttcactctcaccatcagcggcgcgcagtgtgacgatgctgccacttactactgtgcaggatggagaggtgctaggactgatggtgtagatttcggcggagggaccgaggtggtggtcaaag

SEQ ID NO: 80 - DNA, 359 bp, Oryctolagus cuniculus
caggagcagctggtggagtccgggggaggcctggtccagcctggggcatccctgacactcacctgcacagcctctggattctccttcagtagcagctacgtgatgtgctgggtccgccaggctccagggaaggggctggaatggatcacatgcagtgatactggtattaacacatggtacgcgagctgggcgaaaggccgattcaccatctccaaaacctcgtcgaccacggtgactctgcaaatgaccagtctgacagccgcggacacggccacctatttctgtgcgagacgttatactggcgatacttatttgggaaactttaacttgtggggccagggcaccctggtcaccgtctc

SEQ ID NO: 81 - DNA, 328 bp, Oryctolagus cuniculus
gcccaagtgctgacccagactccagcctcggtgtctgcagctgtgggaggcacagtcaccatcaactgccaggccagtcagagtgtttataagaacaactacttatcctggtatcagcagaaaccagggcagcctcccaagctcctgatctacaaggcttccactctggcatctggggtcccatcgcggttcaagggcagtggatctgggacacagttcactctcaccatcagcgacgtgcagtgtgacgatgctgccacttactactgtgcaggcggttggcgtagtctaaatgctttcggcggagggaccgaggtggtggtcaaag

SEQ ID NO: 82 - DNA, 359 bp, Oryctolagus cuniculus
caggagcagctggaggagtccgggggagacctggtcaagcctggggcatccctgacactcacctgcacagcctctgaattctccttcagtagtagatactgggcatgctgggtccgccaggctccagggaaggggctggagtggagcgcatgcatttatactggtagtattgatgctacttactacgcgagctgggcgaaaggccgattcaccatctccaaaacctcgtcgaccacggtgactctgcaagtgaccagtctgacagccgcggacacggccacctatttctgtgtgagggggagtgatgcctggggtctctactttaacttgtggggccagggcaccctggtcaccgtctc

SEQ ID NO: 83 - DNA, 324 bp, Oryctolagus cuniculus
tgctgacccagactccatcctccgtgtctgcagctgtgggaggcacagtcaccgtcagttgccagtccagtcagagtattcataagaataattacttagcctggtatcagcagaaaccagggcagcctcccaagctcctgatctattttgcatccactctggcatctggggtcccatcgcggttcaaaggcagtggatctgggacacagttcactctcaccatcagtgacctggagtgtgacgatgctgccacttactactgtgcaggcgtttatagtggtcgtatttttgctttcggcggagggaccgaggtggtggtcaaag

SEQ ID NO: 84 - DNA, 323 bp, Oryctolagus cuniculus
cagtcggtggaggagtccgggggtcgcctggtcacgcctgggacacccctgacactcacctgcaaagtctctggattctccctcagtagctatacaatgatctgggtccgccaggctccagggaaggggctggagtggatcgggtacatttatggtggtattggtagcacatggtacgcgagctgggcgaaaggccgattcaccatctccaaaacctcgaccacggtggatctgaaaatcaccagtccgacaaccgaggacacggccacctatttctgtggcagaggggacatctggggccagggcaccctggtcaccgtctc

SEQ ID NO: 85 - DNA, 331 bp, Oryctolagus cuniculus
gtgctgacccagactgcatcccccgtgtctgcggctgtgggaggcacagtcaccatcaattgccagtccagtcagagtgtttataagaaccgcttatcctggtatcagcagaaaccagggcagtctcccaagcgcctgatctatggtgcatccactctggaatctggggtcccatcgcggttcaaaggcagcggatctgggacgcagttcactctcaccatcagcgacgtgcagtgtgacgatgctgccacttactactgtctaggcaattatgattgtagtagtgttgattgtggtgctttcggcggagggaccgaggtggtggtcaaag

SEQ ID NO: 86 - DNA, 341 bp, Oryctolagus cuniculus
cagtcggtggaggagtccgggggtcgcctggtcacgcctgggacacccctgacactcacctgcacagtctctggattctccttcagtgcctactacatgacctgggtccgccaggctccagggaaggggctggaattcatcggagtcactatacctattggccgcacgtactacgcgagctgggcgaaaggccgattcaccatctccaaaacctcgaccacggtgcatctgaaaatcaccagtccgacaaccgaggacacggccgcttatttctgtgccggaggtcctactggtaatagccactttacattgtggggccagggcaccctggtcaccgtctc

SEQ ID NO: 87 - DNA, 331 bp, Oryctolagus cuniculus
gtgatgacccagactccatcttccaagtctgtccctgtgggagacacagttaccatcaattgccaggccagtgagagtgtttataataacaaccaattatcctggtatcagcagaaaccagggcagcctcccaagctcctgatctaccaggcatccaaactggaatctggggtcccatcgcggttcaaaggcagtggatctgggacacagttcactctcaccatcagcgatgtggtgtgtgacgatgctgccacttactactgtgcaggatataaaagtcctactactgatggtattgctttcggcggagggaccgaggtggtggtcaaag

SEQ ID NO: 88 - DNA, 335 bp, Oryctolagus cuniculus
cagtcggtggaggagtccgggggtcgcctggtcacgcctgggacacccctgacactcacctgcacagtctctggattctccctcagtagctatgcaatgagctgggtccgccaggctccagggaaggggctggaatggatcggattcatttataattatgaaacatactacgcgaactgggcgaaaggccgattcaccatctccaaaacctcgacctcggtggttctgaaaatcaccagtccgacaaccgacgacacggccacctatttctgtgccagagatatttttcgtactaatactaacttgtggggccagggcaccctggtcaccgtctc

SEQ ID NO: 89 - DNA, 334 bp, Oryctolagus cuniculus
gtgctgacccagactgcatcgcccgtgtctgcagttgtgggaagcacagtcaccatcaattgccaggccagtcagagtgtttataagaacaacggcttatcctggtatcagcagaaaccagggcagcctcccaaaggcctgatctcttttacatcgactctggcatctggggtctcatcgcggttcaaaggcagtggatctgggacacagtttactctcaccatcagcgacgtgcagtgtgacgatgctgccacttactactgtctaggcggttatgattgtagtagtcgtgtttgtggtgctttcggcggagggaccgaggtggtggtcaaag

SEQ ID NO: 90 - DNA, 341 bp, Oryctolagus cuniculus
cagtcggtggaggagtccgggggtcgcctggtcacgcctgggacacccctgacactcacctgcaccgtctctggattcaccttcagtgcctactacatgagctgggtccgccaggctccagggaaggggctggaatacatcggagtcaccattcctagaggtatcacatattacgcgaactgggcgaaaggccgattcaccatctccaaaacctcgaccacggtggatctgagaatcaccagtccgacaaccgaggacacggccacttatttctgtgccggaggtcctactggtaatagccactttacattgtggggccagggcaccctggtcaccgtctc

SEQ ID NO: 91 - DNA, 331 bp, Oryctolagus cuniculus
gtgatgacccagactccatcttccaagtctgtccctgtgggagacacagtcaccatcaattgccaggccagtgagagtgtttatgcgaacaaccaattatcctggtatcagcagaaacaagggcagcctcccaagctcctgatctaccaggcatccaaactggaatctggggtcccatcgcggttcagtggcagtggatctgggacacagttcgctctcaccatcaccgacgtgcagtgtgacgatgctgccacttactactgttcaggatatgtgcgtactactactgatggtattgctttcggcggagggaccgaggtggtggtcaaag

SEQ ID NO: 92 - DNA, 341 bp, Oryctolagus cuniculus
cagtcggtggaggagtccgggggtcgcctggtcacgcctgggacacccctgacactcacctgcacagtctctggattctccctcattagatactacgtgacctgggtccgccaggctccagggaaggggctggaatacatcggagtcactattcccaggggtatcacatactacgcgagctgggcgaaaggccgattcaccatctccaaaacctcgaccacggtggacctgaaaatcaccagtccgacaaccgaggacacggccacttatttctgtgccggaggtcctactggtaatagccactttacattgtggggccagggcaccctggtcaccgtctc

SEQ ID NO: 93 - DNA, 331 bp, Oryctolagus cuniculus
gtgatgacccagaccccatcttccaagtctgtccctgtgggagacacagtcaccatcaattgccaggccagtgagagtgtttataataacaaccaattatcttggtatcagcagaaaccagggcagcctcccaagctcctgatctaccaggcatccaaactggaatctggggtcccatcgcatttcagtggcagtggatctgggacacagttcactctcaccatcagcgacgtgcagtgtgacgatgctgccacttactactgttcaggatataaaagtactactactgatggtattgctttcggcggagggaccgaggtggtggtcaaag

SEQ ID NO: 94 - DNA, 350 bp, Oryctolagus cuniculus
caggagcagttggaggagtccgggggagacctggtcaagcctggggcatccctgacactcacctgcaaagcctctggattctccttcagtagcagctacgtgatgtgctgggtccgccaggctccagggaaggggctggagtggatcgcatgcatttattttgctggtattgatactatctactacgcgagctgggcgaaaggccgattcaccatctccaaaacctcgtcgaccacggtgactctgcaaatgaccagtctgacagccgcggacacggccacctatttctgtgcgagaaatcctattggtagtgtgaatttgtggggccagggcaccctggtcaccgtctc

SEQ ID NO: 95 - DNA, 332 bp, Oryctolagus cuniculus
cgtgatgacccagactccatcttccaagtctgtccctgtgggaggcacagtcaccatcaattgccaggccagtcagagtgtttatagtaacaaccgcttatcctggtttcagcagaaaccagggcagcctcccaagctcctgatctattatttatccactctggcatctggggtcccatcgcggttcaaaggcagtggatctgggacacagttcactctcaccatcagcgacgtgcagtgtgacgatgctgccacttactactgtgcaggatggagaggtgccactactgatggtgttgatttcggcggagggaccgaggtggtggtcaaag

SEQ ID NO: 96 - DNA, 353 bp, Oryctolagus cuniculus
caggagcagctggaggagtccgggggagacctggtcacgcctggaggaaccctgacactcacctgcaaagcctctggattctccttcaatagcaactacgtgatgtgctgggtccgccaggctccagggaaggggctggagtggatcgcatgcatttggtttgctggtagtggtgatgttatctactatgcgagctgggcgaaaggccgattcaccatctccaaaacctcgtcgaccacggtgactctgcaaatgaccagtctgacagccgcggacacggccacccatttctgtgcgagaaatcctgttggtagtgtgaacttgtggggccagggcaccctggtcaccgtctc

SEQ ID NO: 97 - DNA, 332 bp, Oryctolagus cuniculus
cgtgatgacccagactccatcttccaagtctgtccctgtgggaggcacagtcaccatcaattgccaggccagtgagagtgtttatattaacaaccgcttatcctggtttcagcagaaaccagggcaacctcccaagctcctgatctattatgcatccactctggcatctggggtcccatcgcggttcaaaggcagtggatctgggacacagttcactctcaccatcagcggcgtgcagtgtgacgatgctgccacttactactgtgcaggatggaggggtgctactactgatggtattgatttcggcggagggaccgaggtggtggtcaaag

SEQ ID NO: 98 - protein, 114 aa, Oryctolagus cuniculus
QSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVS

SEQ ID NO: 99 - protein, 110 aa, Oryctolagus cuniculus
VMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVK

SEQ ID NO: 100 - protein, 118 aa, Oryctolagus cuniculus
QEQLEESGGDLVKPEGSLTLTCTASGFSFSSNYVMCWVRQAPGKGLEWVACIWFAGIVDTTYYATWAKGRFTISKTSSTTVTLQMTSLTAADTATYFCARNPVGSVNLWGQGTLVTVS

SEQ ID NO: 101 - protein, 110 aa, Oryctolagus cuniculus
VMTQTPSSKSVPVGGSVTINCQASESVYSNNRLSWYQQKPGQPPKLLIYYASTLESGVPSRFKGSGFGTHFTLTISGAQCDDAATYYCAGWRGARTDGVDFGGGTEVVVK

SEQ ID NO: 102 - protein, 120 aa, Oryctolagus cuniculus
QEQLVESGGGLVQPGASLTLTCTASGFSFSSSYVMCWVRQAPGKGLEWITCSDTGINTWYASWAKGRFTISKTSSTTVTLQMTSLTAADTATYFCARRYTGDTYLGNFNLWGQGTLVTVS

SEQ ID NO: 103 - protein, 109 aa, Oryctolagus cuniculus
AQVLTQTPASVSAAVGGTVTINCQASQSVYKNNYLSWYQQKPGQPPKLLIYKASTLASGVPSRFKGSGSGTQFTLTISDVQCDDAATYYCAGGWRSLNAFGGGTEVVVK

SEQ ID NO: 104 - protein, 120 aa, Oryctolagus cuniculus
QEQLEESGGDLVKPGASLTLTCTASEFSFSSRYWACWVRQAPGKGLEWSACIYTGSIDATYYASWAKGRFTISKTSSTTVTLQVTSLTAADTATYFCVRGSDAWGLYFNLWGQGTLVTVS

SEQ ID NO: 105 - protein, 107 aa, Oryctolagus cuniculus
LTQTPSSVSAAVGGTVTVSCQSSQSIHKNNYLAWYQQKPGQPPKLLIYFASTLASGVPSRFKGSGSGTQFTLTISDLECDDAATYYCAGVYSGRIFAFGGGTEVVVK

SEQ ID NO: 106 - protein, 108 aa, Oryctolagus cuniculus
QSVEESGGRLVTPGTPLTLTCKVSGFSLSSYTMIWVRQAPGKGLEWIGYIYGGIGSTWYASWAKGRFTISKTSTTVDLKITSPTTEDTATYFCGRGDIWGQGTLVTVS

SEQ ID NO: 107 - protein, 110 aa, Oryctolagus cuniculus
VLTQTASPVSAAVGGTVTINCQSSQSVYKNRLSWYQQKPGQSPKRLIYGASTLESGVPSRFKGSGSGTQFTLTISDVQCDDAATYYCLGNYDCSSVDCGAFGGGTEVVVK

SEQ ID NO: 108 - protein, 114 aa, Oryctolagus cuniculus
QSVEESGGRLVTPGTPLTLTCTVSGFSFSAYYMTWVRQAPGKGLEFIGVTIPIGRTYYASWAKGRFTISKTSTTVHLKITSPTTEDTAAYFCAGGPTGNSHFTLWGQGTLVTVS

SEQ ID NO: 109 - protein, 110 aa, Oryctolagus cuniculus
VMTQTPSSKSVPVGDTVTINCQASESVYNNNQLSWYQQKPGQPPKLLIYQASKLESGVPSRFKGSGSGTQFTLTISDVVCDDAATYYCAGYKSPTTDGIAFGGGTEVVVK

SEQ ID NO: 110 - protein, 112 aa, Oryctolagus cuniculus
QSVEESGGRLVTPGTPLTLTCTVSGFSLSSYAMSWVRQAPGKGLEWIGFIYNYETYYANWAKGRFTISKTSTSVVLKITSPTTDDTATYFCARDIFRTNTNLWGQGTLVTVS

SEQ ID NO: 111 - protein, 111 aa, Oryctolagus cuniculus
VLTQTASPVSAVVGSTVTINCQASQSVYKNNGLSWYQQKPGQPPKGLISFTSTLASGVSSRFKGSGSGTQFTLTISDVQCDDAATYYCLGGYDCSSRVCGAFGGGTEVVVK

SEQ ID NO: 112 - protein, 114 aa, Oryctolagus cuniculus
QSVEESGGRLVTPGTPLTLTCTVSGFTFSAYYMSWVRQAPGKGLEYIGVTIPRGITYYANWAKGRFTISKTSTTVDLRITSPTTEDTATYFCAGGPTGNSHFTLWGQGTLVTVS

SEQ ID NO: 113 - protein, 110 aa, Oryctolagus cuniculus
VMTQTPSSKSVPVGDTVTINCQASESVYANNQLSWYQQKQGQPPKLLIYQASKLESGVPSRFSGSGSGTQFALTITDVQCDDAATYYCSGYVRTTTDGIAFGGGTEVVVK

SEQ ID NO: 114 - protein, 114 aa, Oryctolagus cuniculus
QSVEESGGRLVTPGTPLTLTCTVSGFSLIRYYVTWVRQAPGKGLEYIGVTIPRGITYYASWAKGRFTISKTSTTVDLKITSPTTEDTATYFCAGGPTGNSHFTLWGQGTLVTVS

SEQ ID NO: 115 - protein, 110 aa, Oryctolagus cuniculus
VMTQTPSSKSVPVGDTVTINCQASESVYNNNQLSWYQQKPGQPPKLLIYQASKLESGVPSHFSGSGSGTQFTLTISDVQCDDAATYYCSGYKSTTTDGIAFGGGTEVVVK

SEQ ID NO: 116 - protein, 117 aa, Oryctolagus cuniculus
QEQLEESGGDLVKPGASLTLTCKASGFSFSSSYVMCWVRQAPGKGLEWIACIYFAGIDTIYYASWAKGRFTISKTSSTTVTLQMTSLTAADTATYFCARNPIGSVNLWGQGTLVTVS

SEQ ID NO: 117 - protein, 110 aa, Oryctolagus cuniculus
VMTQTPSSKSVPVGGTVTINCQASQSVYSNNRLSWFQQKPGQPPKLLIYYLSTLASGVPSRFKGSGSGTQFTLTISDVQCDDAATYYCAGWRGATTDGVDFGGGTEVVVK

SEQ ID NO: 118 - protein, 118 aa, Oryctolagus cuniculus
QEQLEESGGDLVTPGGTLTLTCKASGFSFNSNYVMCWVRQAPGKGLEWIACIWFAGSGDVIYYASWAKGRFTISKTSSTTVTLQMTSLTAADTATHFCARNPVGSVNLWGQGTLVTVS

SEQ ID NO: 119 - protein, 110 aa, Oryctolagus cuniculus
VMTQTPSSKSVPVGGTVTINCQASESVYINNRLSWFQQKPGQPPKLLIYYASTLASGVPSRFKGSGSGTQFTLTISGVQCDDAATYYCAGWRGATTDGIDFGGGTEVVVK

SEQ ID NO: 120 - protein, 8 aa, Oryctolagus cuniculus
GFSLSSYN

SEQ ID NO: 121 - protein, 7 aa, Oryctolagus cuniculus
IGTGGIT

SEQ ID NO: 122 - protein, 12 aa, Oryctolagus cuniculus
VRSSGSDWWFHI

SEQ ID NO: 123 - protein, 8 aa, Oryctolagus cuniculus
QSVYNNNN

SEQ ID NO: 124 - protein, 3 aa, Oryctolagus cuniculus
RAS

SEQ ID NO: 125 - protein, 10 aa, Oryctolagus cuniculus
QGYFSGFITT

SEQ ID NO: 126 - DNA, 341 bp, Oryctolagus cuniculus
cagtcgctggaggagtccgggggtcgcctggtcacgcctgggacacccctgacactcacctgcacagtctctggattctccctcagtagctacaacatgggctgggtccgccaggctccaggggaggggctggagtggatcggagtcattggtactggtggtatcacacactacgcgacctgggcaaaaggccgagtcgccatctccagaacctcgaccacggtgggtctgcgaatgaccagtccgacaaccgaggacacggccacctatttctgtgtcagatctagtggtagtgattggtggtttcacatctggggccagggcaccctggtcaccgtctc

SEQ ID NO: 127 - DNA, 325 bp, Oryctolagus cuniculus
gtgctgacccagactacatcgcccgtgtctgcagctgtgggaggcacagtcaccatcagttgccagtccagtcagagtgtttataataacaacaacttagcctggtttcagcagaaaccagggcagcctcccaagctcctgatctacagggcatccaatctgccatctggtgtcccatcgcggttcagaggcagtggatctgggtcacagttcactctcacaatcagcgaagtacagtgtgacgatgctgccacttactactgtcaaggctattttagtggatttatcactactttcggcggagggaccgaggtggtggtcaaag

SEQ ID NO: 128 - protein, 114 aa, Oryctolagus cuniculus
QSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVS

SEQ ID NO: 129 - protein, 108 aa, Oryctolagus cuniculus
VLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVK

SEQ ID NO: 130 - protein, 8 aa, Mus musculus
GFTFSSYY

SEQ ID NO: 131 - protein, 8 aa, Mus Musculus
INSNGGST

SEQ ID NO: 132 - protein, 9 aa, Mus Musculus
VRQNYEGAY

SEQ ID NO: 133 - protein, 11 aa, Mus Musculus
QSIVHSNGNTY

SEQ ID NO: 134 - protein, 3 aa, Mus Musculus
KVS

SEQ ID NO: 135 - protein, 9 aa, Mus Musculus
FQSSHVPWT

SEQ ID NO: 136 - DNA, 348 bp, Mus Musculus
gacgtgaagctcgtggagtctgggggaggcttagtgaagcttggagggtccctgaaactctcctgtgcagcctctggattcactttcagtagctattacatgtcttgggttcgccagactccagagaagaggctggagttggtcgcaaccattaatagtaatggtggtagcacctactatccagacactgtgaagggccgaatcaccatctccagagacaatgccaagaacaccctgcagttgcaaatgagcagtctgaggtctgaggacacagccttgtattactgtgtaagacaaaactacgagggggcttactggggccaagggactctggtcactgtctctgca

SEQ ID NO: 137 - DNA, 336 bp, Mus Musculus
gatgttttgatgacccaaactccactctccctgcctgtcactcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaagttcacatgttccgtggacgttcggtggaggcaccaagctggaaatcaaa

SEQ ID NO: 138 - protein, 116 aa, Mus Musculus
DVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSA

SEQ ID NO: 139 - protein, 112 aa, Mus Musculus
DVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTFGGGTKLEIK

SEQ ID NO: 140 - protein, 8 aa, Mus Musculus
GFTFSSYY

SEQ ID NO: 141 - protein, 8 aa, Mus Musculus
INTNGGST

SEQ ID NO: 142 - protein, 9 aa, Mus Musculus
VRQNYEGAY

SEQ ID NO: 143 - protein, 11 aa, Mus Musculus
QSIVHSNGNTY

SEQ ID NO: 144 - protein, 3 aa, Mus Musculus
KVS

SEQ ID NO: 145 - protein, 9 aa, Mus Musculus
FQSSHVPWT

SEQ ID NO: 146 - DNA, 348 bp, Mus Musculus
gacgtgaagctcgtggagtctgggggagacttagtgaagcttggagggtccctgaaactctcctgtgcagcctctggattcactttcagtagctattacatgtcttgggttcgccagactccagagaagaggctggagttggtcgcagtcattaatactaatggtggtagcacctactatccagacactgtgaagggccgattcaccatctccagagacaatgccaagaacaccctgtacctgcaaatgagcagtctgaagtctgaggacacagccttgtattactgtgtaagacaaaactacgagggggcttactggggccaagggactctggtcactgtctctgca

SEQ ID NO: 147 - DNA, 336 bp, Mus Musculus
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaagttcacatgttccgtggacgttcggtggaggcaccaagctggaaatcaaa

SEQ ID NO: 148 - protein, 116 aa, Mus Musculus
DVKLVESGGDLVKLGGSLKLSCAASGFTFSSYYMSWVRQTPEKRLELVAVINTNGGSTYYPDTVKGRFTISRDNAKNTLYLQMSSLKSEDTALYYCVRQNYEGAYWGQGTLVTVSA

SEQ ID NO: 149 - protein, 112 aa, Mus Musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQSSHVPWTFGGGTKLEIK

SEQ ID NO: 150-159 - skipped (10 entries)

SEQ ID NO: 160 - protein, 19 aa, Artificial Sequence
  Synthetic Construct
MNFGLRLILLVLVLKGVLC

SEQ ID NO: 161 - protein, 20 aa, Artificial Sequence
  Synthetic Construct
METGLRWLLLVAVLKGVQCQ

SEQ ID NO: 162 - protein, 19 aa, Artificial Sequence
  Synthetic Construct
MELGLSWIFLLAILKGVQC

SEQ ID NO: 163 - protein, 19 aa, Artificial Sequence
  Synthetic Construct
MELGLRWVFLVAILEGVQC

SEQ ID NO: 164 - protein, 19 aa, Artificial Sequence
  Synthetic Construct
MKHLWFFLLLVAAPRWVLS

SEQ ID NO: 165 - protein, 19 aa, Artificial Sequence
  Synthetic Construct
MDWTWRILFLVAAATGAHS

SEQ ID NO: 166 - protein, 19 aa, Artificial Sequence
  Synthetic Construct
MDWTWRFLFVVAAATGVQS

SEQ ID NO: 167 - protein, 19 aa, Artificial Sequence
  Synthetic Construct
MEFGLSWLFLVAILKGVQC

SEQ ID NO: 168 - protein, 19 aa, Artificial Sequence
  Synthetic Construct
MEFGLSWVFLVALFRGVQC

SEQ ID NO: 169 - protein, 26 aa, Artificial Sequence
  Synthetic Construct
MDLLHKNMKHLWFFLLLVAAPRWVLS

SEQ ID NO: 170 - protein, 19 aa, Artificial Sequence
  Synthetic Construct
MKLPVRLLVLMFWIPASSS

SEQ ID NO: 171 - protein, 22 aa, Artificial Sequence
  Synthetic Construct
MDMRVPAQLLGLLLLWLSGARC

SEQ ID NO: 172 - protein, 22 aa, Artificial Sequence
  Synthetic Construct
MKYLLPTAAAGLLLLAAQPAMA

SEQ ID NO: 173 - protein, 4 aa, Artificial Sequence
  Synthetic Construct
YPTL

SEQ ID NO: 174 - protein, 10 aa, Artificial Sequence
  Synthetic Construct
KSIRSGYEVM

SEQ ID NO: 175 - protein, 23 aa, Artificial Sequence
  Synthetic Construct
RWRKSHSSSYTPLSGSTYPEGRH

SEQ ID NO: 176 - protein, 4 aa, Artificial Sequence
  Synthetic Construct
  misc_feature (3)..(3): Xaa can be any naturally occurring amino acid
NPXY

SEQ ID NO: 177 - protein, 4 aa, Artificial Sequence
  Synthetic Construct
  misc_feature (2)..(3): Xaa can be any naturally occurring amino acid
  MISC_FEATURE (4)..(4): Xaa is Trp, Tyr, Val, Ile, Leu, Phe or Met
YXXX

SEQ ID NO: 178 - protein, 6 aa, Artificial Sequence
  Synthetic Construct
  MISC_FEATURE (1)..(1): Xaa is either Asp or Glu
  misc_feature (2)..(4): Xaa can be any naturally occurring amino acid
  MISC_FEATURE (6)..(6): Xaa is either Leu or Ile
XXXXLX

SEQ ID NO: 179 - protein, 5 aa, Artificial Sequence
  Synthetic Construct
  misc_feature (2)..(3): Xaa can be any naturally occurring amino acid
DXXLL

SEQ ID NO: 180 - protein, 15 aa, Artificial Sequence
  Synthetic Construct
GGGGSGGGGSGGGGS

SEQ ID NO: 181 - protein, 20 aa, Artificial Sequence
  Synthetic Construct
GGGGSGGGGSGGGGSGGGGS

SEQ ID NO: 182 - protein, 20 aa, Artificial Sequence
  Synthetic Construct
GSTGGGGSGKPGSGEGGGGS

SEQ ID NO: 183 - protein, 15 aa, Artificial Sequence
  Synthetic Construct
ESGRSGGGGSGGGGS

SEQ ID NO: 184 - protein, 14 aa, Artificial Sequence
  Synthetic Construct
EGKSSGSGSESKST

SEQ ID NO: 185 - protein, 15 aa, Artificial Sequence
  Synthetic Construct
EGKSSGSGSESKSTQ

SEQ ID NO: 186 - protein, 14 aa, Artificial Sequence
  Synthetic Construct
EGKSSGSGSESKVD

SEQ ID NO: 187 - protein, 14 aa, Artificial Sequence
  Synthetic Construct
GSTSGSGKSSEGKG

SEQ ID NO: 188 - protein, 18 aa, Artificial Sequence
  Synthetic Construct
KESGSVSSEQLAQFRSLD

SEQ ID NO: 189 - protein, 16 aa, Artificial Sequence
  Synthetic Construct
ESGSVSSEELAFRSLD

SEQ ID NO: 190 - protein, 5 aa, Artificial Sequence
  Synthetic Construct
GGGGS

SEQ ID NO: 191 - protein, 11 aa, Artificial Sequence
  Synthetic Construct
YGRKKRRQRRR

SEQ ID NO: 192 - protein, 13 aa, Artificial Sequence
  Synthetic Construct
CYGRKKRRQRRRC

SEQ ID NO: 193 - protein, 31 aa, Artificial Sequence
  Synthetic Construct
GDIMGEWGNEIFGAIAGFLGYGRKKRRQRRR

SEQ ID NO: 194 - protein, 8 aa, Artificial Sequence
  Synthetic Construct
RRRRRRRR

SEQ ID NO: 195 - protein, 10 aa, Artificial Sequence
  Synthetic Construct
CRRRRRRRRC

SEQ ID NO: 196 - protein, 16 aa, Artificial Sequence
  Synthetic Construct
RQIKIWFQNRRMKWKK

SEQ ID NO: 197 - protein, 27 aa, Artificial Sequence
  Synthetic Construct
GWTLNSAGYLLGKINLKALAALAKKIL

SEQ ID NO: 198-201 - skipped (4 entries)

SEQ ID NO: 202 - DNA, 807 bp, Artificial Sequence
  Synthetic Construct
atggactacaaggacgacgacgacaaggacgtgaagctggtggaaagcggaggaggactggtgaagctgggcggaagcctgaagctgtcttgcgccgccagcggcttcacctttagcagctactacatgtcttgggtccggcagaccccagagaagagactggagctggtggccaccatcaacagcaacggaggcagcacctactaccccgataccgtgaagggccggatcaccatcagcagggacaacgccaagaacaccctgcagctgcagatgagcagcctgagaagcgaggacaccgccctgtactattgcgtgcggcagaactacgagggagcctattggggacagggaacactggtgacagtgtccgcaggaggaggaggaagcggaggaggaggaagcggaggaggaggatctgacgtgctgatgacccagacccctctgtctctgccagtgacactgggagaccaggcttctatctcttgccggagcagccagagcatcgtgcacagcaacggcaacacctacctcgagtggtacctgcagaagccaggacagagccccaagctgctgatctacaaggtgtccaaccggttcagcggcgtgccagatagattcagcggaagcggaagcggcaccgacttcaccctgaagatctctagagtggaagccgaggatctgggcgtgtactactgcttccagagcagccacgtgccttggacatttggcggcggcacaaagctggagatcaagggcggcggcggcagcaagagcatccggagcggctacgaggtcatgtgataa

SEQ ID NO: 203 - protein, 267 aa, Artificial Sequence
  Synthetic Construct
MDYKDDDDKDVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSAGGGGSGGGGSGGGGSDVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTFGGGTKLEIKGGGGSKSIRSGYEVM

SEQ ID NO: 204 - DNA, 795 bp, Artificial Sequence
  Synthetic Construct
atggactacaaggacgacgacgacaagcagagcgtggaggaaagcggaggaagactggtgacaccaggcacccctctgaccctgacttgtacagtgtccggcttcagcctgagcaggtactacatgacttgggtccggcaggcaccaggaaagggcctggagtacatcggcgtgatcatcccaggaggcaccacatactacgcctcttgggccaagggcaggttcaccatcagcaagaccagcaccaccgtggacctgaggatcacaagccctaccaccgaggacaccgccacctacttttgtgccggaggacctacaggcaatagccacttcaccctctggggacagggaacactggtgacagtgtccggaggaggaggaagcggaggaggaggaagcggaggaggaggaagcgtgatgacacagacccctagcagcaagagcgtgccagtgggcggaaccgtgaccatcaattgccaggccagcgagagcgtgtacaacaacaaccacctgtcttggtaccagcagaagagcggccagcctcctaagctgctgatctacgaggccagcaagctggagagcggagtgcctcccagattcaaaggcagcggaagcggcacccagtttaccctgaccatctcagacgtcgtctgcgacgacgccgctacctactattgcagcggctacaagagagtgaccaccgacggcatcgcctttggaggaggcacagaagtggtggtgaaaggcggaggaggcagcaagagcatcagaagcggctacgaggtcatgtgataa

SEQ ID NO: 205 - protein, 263 aa, Artificial Sequence
  Synthetic Construct
MDYKDDDDKQSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVSGGGGSGGGGSGGGGSVMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVKGGGGSKSIRSGYEVM

SEQ ID NO: 206 - DNA, 789 bp, Artificial Sequence
  Synthetic Construct
atggactacaaggacgacgacgacaagcagagcctggaggaaagcggaggaagactggtgacaccaggcacccctctgaccctgacttgtacagtgtccggcttcagcctgagcagctacaacatgggttgggtgcgacaggctccaggagaaggactcgagtggatcggcgtgatcggaacaggaggcatcacccactacgccacttgggctaagggcagagtggccatcagcagaaccagcaccaccgtgggactgagaatgaccagccctaccaccgaggacaccgccacctacttttgcgtgagatctagcggcagcgattggtggttccacatttgggggcagggcacactggtgacagtgtccggaggaggaggaagcggaggaggaggatctggaggaggaggaagcgtgctcacacagacaacaagcccagtgtccgccgcagtgggaggaaccgtgaccatctcttgccagagcagccagagcgtgtacaacaacaacaacctggcttggttccagcagaagcccggacagcctcctaagctgctgatctacagggccagcaacctgcctagcggcgtgcctagcagattcagaggaagcggaagcggcagccagtttaccctgaccatcagcgaagtccagtgcgacgacgcagccacctactattgccagggctacttcagcggctttatcaccacctttggcggcggaaccgaagtggtggtgaaaggcggaggaggaagcaagagcatccggagcggctacgaagtgatgtgataa

SEQ ID NO: 207 - protein, 261 aa, Artificial Sequence
  Synthetic Construct
MDYKDDDDKQSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVSGGGGSGGGGSGGGGSVLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVKGGGGSKSIRSGYEVM

SEQ ID NO: 208 - DNA, 807 bp, Artificial Sequence
  Synthetic Construct
atggactacaaggacgacgacgacaagcaggagcagctggaagagagcggaggagatctggtgaagccagagggcagcctgacactgacttgtacagccagcggcttcagcttcagcagcaactacgtcatgtgttgggtccggcaggcaccaggaaaaggactggagtgggtggcttgcatttggttcgccggcatcgtggacaccacctactacgccacttgggccaagggacggttcaccatcagcaagaccagcagcaccaccgtgaccctgcagatgacatctctgacagccgccgacaccgctacctacttttgcgcccggaacccagtgggatcagtcaacctctggggacagggaacactggtgaccgtgtcaggaggaggaggatctggaggaggaggaagcggaggaggaggcagcgtgatgacacagacacctagcagcaaaagcgtgccagtgggaggaagcgtgaccatcaattgccaggccagcgagagcgtgtacagcaacaaccggctgtcttggtaccagcagaagccaggccagcctcctaagctgctgatctactacgcctctaccctggagagcggagtgcctagcagattcaagggcagcggctttggcacccacttcacactgaccatcagcggagcccagtgcgacgacgcagccacctactattgcgcaggctggagaggagctagaacagacggagtggacttcggcggaggaacagaagtggtggtgaaaggcggcggcggctctaaaagcatccggagcggctacgaggtcatgtgataa

SEQ ID NO: 209 - protein, 267 aa, Artificial Sequence
  Synthetic Construct
MDYKDDDDKQEQLEESGGDLVKPEGSLTLTCTASGFSFSSNYVMCWVRQAPGKGLEWVACIWFAGIVDTTYYATWAKGRFTISKTSSTTVTLQMTSLTAADTATYFCARNPVGSVNLWGQGTLVTVSGGGGSGGGGSGGGGSVMTQTPSSKSVPVGGSVTINCQASESVYSNNRLSWYQQKPGQPPKLLIYYASTLESGVPSRFKGSGFGTHFTLTISGAQCDDAATYYCAGWRGARTDGVDFGGGTEVVVKGGGGSKSIRSGYEVM

SEQ ID NO: 210-211 - skipped (2 entries)

SEQ ID NO: 212 - DNA, 828 bp, Artificial Sequence
  Synthetic Construct
atggacgtgaagctcgtggagtctgggggaggcttagtgaagcttggagggtccctgaaactctcctgtgcagcctctggattcactttcagtagctattacatgtcttgggttcgccagactccagagaagaggctggagttggtcgcaaccattaatagtaatggtggtagcacctactatccagacactgtgaagggccgaatcaccatctccagagacaatgccaagaacaccctgcagttgcaaatgagcagtctgaggtctgaggacacagccttgtattactgtgtaagacaaaactacgagggggcttactggggccaagggactctggtcactgtctctgcaggcggaggtggtagcggaggaggtggcagcgggggaggtggctcagatgttttgatgacccaaactccactctccctgcctgtcactcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaagttcacatgttccgtggacgttcggtggaggcaccaagctggaaatcaaagattacaaggatgacgacgataagcgctggcgtaaatctcattcatcctcctacacccccttgtccggttctacctaccccgaagggcgccattag

SEQ ID NO: 213 - protein, 275 aa, Artificial Sequence
  Synthetic Construct
MDVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSAGGGGSGGGGSGGGGSDVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTFGGGTKLEIKDYKDDDDKRWRKSHSSSYTPLSGSTYPEGRH

SEQ ID NO: 214 - DNA, 819 bp, Artificial Sequence
  Synthetic Construct
atgcagtcggtggaggagtccgggggtcgcctggtcacgcctgggacacccctgacactcacctgcacagtctctggattctccctcagtaggtactacatgacctgggtccgccaggctccagggaaggggctggaatacatcggggtcattattcctggtggtaccacatactacgcgagctgggcgaaaggccgattcaccatctccaaaacctcgaccacggtggatctgagaatcaccagtccgacaaccgaggacacggccacttatttctgtgccggaggtcctactggtaacagccactttacattgtggggccagggcaccctggtcaccgtctcctcaggcggaggtggtagcggaggaggtggcagcgggggaggtggctcagtgatgacccagactccatcttccaagtctgtccctgtgggaggcacagtcaccatcaattgccaggccagtgagagtgtttataataacaaccacttatcctggtatcagcagaaatcagggcagcctcccaagctcctgatctacgaagcatccaaactggaatctggggtcccaccgcggttcaaaggcagtggatctgggacacagttcactctcaccatcagcgatgtggtgtgtgacgatgctgccacttactactgttcaggatataaacgtgttactactgatggtattgctttcggcggagggaccgaggtggtggtcaaagattacaaggatgacgacgataagcgctggcgtaaatctcattcatcctcctacacccccttgtccggttctacctaccccgaagggcgccattag

SEQ ID NO: 215 - protein, 272 aa, Artificial Sequence
  Synthetic Construct
MQSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVSSGGGGSGGGGSGGGGSVMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVKDYKDDDDKRWRKSHSSSYTPLSGSTYPEGRH

SEQ ID NO: 216 - DNA, 813 bp, Artificial Sequence
  Synthetic Construct
atgcagtcgctggaggagtccgggggtcgcctggtcacgcctgggacacccctgacactcacctgcacagtctctggattctccctcagtagctacaacatgggctgggtccgccaggctccaggggaggggctggagtggatcggagtcattggtactggtggtatcacacactacgcgacctgggcaaaaggccgagtcgccatctccagaacctcgaccacggtgggtctgcgaatgaccagtccgacaaccgaggacacggccacctatttctgtgtcagatctagtggtagtgattggtggtttcacatctggggccagggcaccctggtcaccgtctcctcaggcggaggtggtagcggaggaggtggcagcgggggaggtggctcagtgctgacccagactacatcgcccgtgtctgcagctgtgggaggcacagtcaccatcagttgccagtccagtcagagtgtttataataacaacaacttagcctggtttcagcagaaaccagggcagcctcccaagctcctgatctacagggcatccaatctgccatctggtgtcccatcgcggttcagaggcagtggatctgggtcacagttcactctcacaatcagcgaagtacagtgtgacgatgctgccacttactactgtcaaggctattttagtggatttatcactactttcggcggagggaccgaggtggtggtcaaagattacaaggatgacgacgataagcgctggcgtaaatctcattcatcctcctacacccccttgtccggttctacctaccccgaagggcgccattag

SEQ ID NO: 217 - protein, 270 aa, Artificial Sequence
  Synthetic Construct
MQSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVSSGGGGSGGGGSGGGGSVLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVKDYKDDDDKRWRKSHSSSYTPLSGSTYPEGRH

SEQ ID NO: 218 - DNA, 831 bp, Artificial Sequence
  Synthetic Construct
atgcaggagcagctggaggagtccgggggagacctggtcaagcctgagggatccctgacactcacctgcacagcctctggattctccttcagtagcaactacgtgatgtgctgggtccgccaggctccagggaaggggctggagtgggtcgcatgcatttggtttgctggtattgttgatactacttactacgcgacctgggcgaaaggccgattcaccatctccaaaacctcgtcgaccacggtgactctgcaaatgaccagtctgacagccgcggacacggccacctatttctgtgcgagaaatcctgttggtagtgtgaacttgtggggccagggcaccctggtcaccgtctcctcaggcggaggtggtagcggaggaggtggcagcgggggaggtggctcagtgatgacccagactccatcttccaagtctgtccctgtgggaggctcagtcaccatcaattgccaggccagtgagagtgtttatagtaacaaccgcttatcctggtatcagcagaaaccagggcagcctcctaagctcctgatctattatgcatccactctggaatctggggtcccatcgcggttcaaaggcagtggatttgggacacacttcactctcaccatcagcggcgcgcagtgtgacgatgctgccacttactactgtgcaggatggagaggtgctaggactgatggtgtagatttcggcggagggaccgaggtggtggtcaaagattacaaggatgacgacgataagcgctggcgtaaatctcattcatcctcctacacccccttgtccggttctacctaccccgaagggcgccattag

SEQ ID NO: 219 - protein, 276 aa, Artificial Sequence
  Synthetic Construct
MQEQLEESGGDLVKPEGSLTLTCTASGFSFSSNYVMCWVRQAPGKGLEWVACIWFAGIVDTTYYATWAKGRFTISKTSSTTVTLQMTSLTAADTATYFCARNPVGSVNLWGQGTLVTVSSGGGGSGGGGSGGGGSVMTQTPSSKSVPVGGSVTINCQASESVYSNNRLSWYQQKPGQPPKLLIYYASTLESGVPSRFKGSGFGTHFTLTISGAQCDDAATYYCAGWRGARTDGVDFGGGTEVVVKDYKDDDDKRWRKSHSSSYTPLSGSTYPEGRH

SEQ ID NO: 220-221 - skipped (2 entries)

SEQ ID NO: 222 - DNA, 771 bp, Artificial Sequence
  Synthetic Construct
atggatgtgctcatgacacaaacacctttgagccttcccgtgacactcggcgaccaggccagtatctcctgcagaagttcccagtctatcgtgcactcaaacggtaatacgtacttggagtggtatctgcaaaaacccggacagtccccaaagctgttgatatacaaggtttctaatcgcttcagcggtgtaccagacagattttctggctccggatcaggaaccgactttacccttaagatttcaagggtggaagccgaggaccttggagtgtactattgcttccaatcaagtcacgtcccatggactgggggcggcaccaagctcgagatcaagggaggtggcggatcaggtggcggaggttctggcggtggcggttctgatgtgaagttggttgaaagtggaggcggtctcgttaaactgggcggaagtctgaaactttcatgtgcagcttccgggttcaccttctctagctattacatgagttgggtacggcagacccccgaaaagcgactggagctggtggctaccataaattctaacggaggatctacgtattaccccgacaccgtgaaggggcgcatcaccatttctcgggacaatgccaaaaatacactgcagttgcagatgtcatcactgcgaagcgaagacaccgcgctgtactactgtgtaagacagaattacgagggcgcatattggggacagggcactctggtcaccgtgtcagctggcagtggagagcagaagctgatctcagaggaggacctgtga

SEQ ID NO: 223 - protein, 256 aa, Artificial Sequence
  Synthetic Construct
MDVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTGGGTKLEIKGGGGSGGGGSGGGGSDVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSAGSGEQKLISEEDL

SEQ ID NO: 224 - DNA, 771 bp, Artificial Sequence
  Synthetic Construct
atggcaatcgtgatgacccagactcctagctccaaaagcgtgcccgtgggtggaactgttacgatcaactgtcaggcgagcgagtccgtgtacaacaacaaccatctttcttggtatcagcagaagtctggacagccgcccaagctgctcatatacgaagcgtcaaagctggaatccggagtgccacctcgctttaagggaagcgggagtggcacccagttcactctgaccataagtgacgttgtttgtgacgacgctgccacatattattgcagtggttataagcgagtcaccacggacggcatcgcctttggaggtggcaccgaggtggttgtgaagggaggtggcggatcaggtggcggaggttctggcggtggcggttctcagagcgtggaggaatcaggtggaagactcgttactccaggtactccgctgaccttgacttgtaccgtgagtggcttttccctgtcccgctactacatgacctgggtgaggcaggcacctggcaaagggctggaatacattggggtgatcatacctggcggaacaacatattacgccagttgggcaaaaggtcgctttaccatttccaaaacatccactactgtggacctcagaatcacatccccaactacagaagacacagccacatacttctgcgcaggaggaccaaccggcaactctcatttcacactctggggccagggcactctggtcaccgtgtcatctggcagtggagagcagaagctgatctcagaggaggacctgtga

SEQ ID NO: 225 - protein, 256 aa, Artificial Sequence
  Synthetic Construct
MAIVMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVKGGGGSGGGGSGGGGSQSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVSSGSGEQKLISEEDL

SEQ ID NO: 226 - DNA, 765 bp, Artificial Sequence
  Synthetic Construct
atggcacaagtgttgacccagactacttcacccgttagtgcagccgtgggaggcaccgttaccattagctgccagtcatcccagtcagtttataataacaataaccttgcctggttccagcagaaaccaggccagccccccaagctcctgatctatagagccagcaatctgccgagcggggttccttccaggttcagagggagtggtagtgggtctcagttcacactcaccatcagcgaggtgcaatgtgatgacgctgctacctactactgccaggggtacttcagcggattcatcacaacctttggaggtggcaccgaggtggttgtgaagggaggtggcggatcaggtggcggaggttctggcggtggcggttctcagagcctggaggaatcaggtggaagacttgttacaccaggtacgcccctgactctgacatgtactgtgagcggattttccctgagtagctataacatgggatgggtcagacaggcccctggtgagggtctcgagtggatcggtgtgatcggtaccggaggtatcactcactatgcaacctgggcaaaaggacgcgtggccatcagcaggaccagcacaactgtaggcctgcggatgactagcccaacaaccgaagacacagcaacatacttttgtgtccggagcagcggttccgattggtggtttcacatctggggacagggaactctggtcaccgtgtcatctggcagtggagagcagaagctgatctcagaggaggacctgtga

SEQ ID NO: 227 - protein, 254 aa, Artificial Sequence
  Synthetic Construct
MAQVLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVKGGGGSGGGGSGGGGSQSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVSSGSGEQKLISEEDL

SEQ ID NO: 228 - DNA, 783 bp, Artificial Sequence
  Synthetic Construct
atggcaatcgtgatgacccagactccttctagtaagagtgtgcctgtgggagggagcgtgacaatcaactgccaggccagcgagtccgtgtatagtaataatcgactctcatggtatcaacagaagcccggccagcctcctaaattgttgatatactatgcaagtaccctcgaaagcggtgttccatccaggtttaaagggagcggctttgggactcacttcactcttacgattagtggagcacaatgtgatgacgcagctacttattattgtgccggttggagaggggctagaaccgatggagtggattttggaggtggcaccgaggtggttgtgaagggaggtggcggatcaggtggcggaggttctggcggtggcggttctcaggaacaactggaggaatcaggtggagatttggtgaagccggaggggtctctgaccctgacatgtaccgcctccgggttcagtttttcctcaaattacgtcatgtgctgggtcagacaggcgcctggaaaggggctggaatgggtggcatgcatttggtttgctggaattgtagataccacctactatgccacgtgggcgaaaggacggttcaccatcagcaagactagtagcacaactgttaccctccagatgacaagcctgaccgcagctgacactgccacttatttctgcgcgcgcaaccctgttggatcagttaatctgtggggacaaggcactctggtcaccgtgtcatctggcagtggagagcagaagctgatctcagaggaggacctgtga

SEQ ID NO: 229 - protein, 260 aa, Artificial Sequence
  Synthetic Construct
MAIVMTQTPSSKSVPVGGSVTINCQASESVYSNNRLSWYQQKPGQPPKLLIYYASTLESGVPSRFKGSGFGTHFTLTISGAQCDDAATYYCAGWRGARTDGVDFGGGTEVVVKGGGGSGGGGSGGGGSQEQLEESGGDLVKPEGSLTLTCTASGFSFSSNYVMCWVRQAPGKGLEWVACIWFAGIVDTTYYATWAKGRFTISKTSSTTVTLQMTSLTAADTATYFCARNPVGSVNLWGQGTLVTVSSGSGEQKLISEEDL

SEQ ID NO: 230-231 - skipped (2 entries)

SEQ ID NO: 232 - DNA, 771 bp, Artificial Sequence
  Synthetic Construct
atggatgtgaagttggttgaaagtggaggcggtctcgttaaactgggcggaagtctgaaactttcatgtgcagcttccgggttcaccttctctagctattacatgagttgggtacggcagacccccgaaaagcgactggagctggtggctaccataaattctaacggaggatctacgtattaccccgacaccgtgaaggggcgcatcaccatttctcgggacaatgccaaaaatacactgcagttgcagatgtcatcactgcgaagcgaagacaccgcgctgtactactgtgtaagacagaattacgagggcgcatattggggacagggcactctggtcaccgtgtcagctggaggtggcggatcaggtggcggaggttctggcggtggcggttctgatgtgctcatgacacaaacacctttgagccttcccgtgacactcggcgaccaggccagtatctcctgcagaagttcccagtctatcgtgcactcaaacggtaatacgtacttggagtggtatctgcaaaaacccggacagtccccaaagctgttgatatacaaggtttctaatcgcttcagcggtgtaccagacagattttctggctccggatcaggaaccgactttacccttaagatttcaagggtggaagccgaggaccttggagtgtactattgcttccaatcaagtcacgtcccatggactgggggcggcaccaagctcgagatcaagggcagtggagagcagaagctgatctcagaggaggacctgtga

SEQ ID NO: 233 - protein, 256 aa, Artificial Sequence
  Synthetic Construct
MDVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSAGGGGSGGGGSGGGGSDVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTGGGTKLEIKGSGEQKLISEEDL

SEQ ID NO: 234 - DNA, 771 bp, Artificial Sequence
  Synthetic Construct
atgcagagcgtggaggaatcaggtggaagactcgttactccaggtactccgctgaccttgacttgtaccgtgagtggcttttccctgtcccgctactacatgacctgggtgaggcaggcacctggcaaagggctggaatacattggggtgatcatacctggcggaacaacatattacgccagttgggcaaaaggtcgctttaccatttccaaaacatccactactgtggacctcagaatcacatccccaactacagaagacacagccacatacttctgcgcaggaggaccaaccggcaactctcatttcacactctggggccagggcactctggtcaccgtgtcatctggaggtggcggatcaggtggcggaggttctggcggtggcggttctgcaatcgtgatgacccagactcctagctccaaaagcgtgcccgtgggtggaactgttacgatcaactgtcaggcgagcgagtccgtgtacaacaacaaccatctttcttggtatcagcagaagtctggacagccgcccaagctgctcatatacgaagcgtcaaagctggaatccggagtgccacctcgctttaagggaagcgggagtggcacccagttcactctgaccataagtgacgttgtttgtgacgacgctgccacatattattgcagtggttataagcgagtcaccacggacggcatcgcctttggaggtggcaccgaggtggttgtgaagggcagtggagagcagaagctgatctcagaggaggacctgtga

SEQ ID NO: 235 - protein, 256 aa, Artificial Sequence
  Synthetic Construct
MQSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVSSGGGGSGGGGSGGGGSAIVMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVKGSGEQKLISEEDL

SEQ ID NO: 236 - DNA, 765 bp, Artificial Sequence
  Synthetic Construct
atgcagagcctggaggaatcaggtggaagacttgttacaccaggtacgcccctgactctgacatgtactgtgagcggattttccctgagtagctataacatgggatgggtcagacaggcccctggtgagggtctcgagtggatcggtgtgatcggtaccggaggtatcactcactatgcaacctgggcaaaaggacgcgtggccatcagcaggaccagcacaactgtaggcctgcggatgactagcccaacaaccgaagacacagcaacatacttttgtgtccggagcagcggttccgattggtggtttcacatctggggacagggaactctggtcaccgtgtcatctggaggtggcggatcaggtggcggaggttctggcggtggcggttctgcacaagtgttgacccagactacttcacccgttagtgcagccgtgggaggcaccgttaccattagctgccagtcatcccagtcagtttataataacaataaccttgcctggttccagcagaaaccaggccagccccccaagctcctgatctatagagccagcaatctgccgagcggggttccttccaggttcagagggagtggtagtgggtctcagttcacactcaccatcagcgaggtgcaatgtgatgacgctgctacctactactgccaggggtacttcagcggattcatcacaacctttggaggtggcaccgaggtggttgtgaagggcagtggagagcagaagctgatctcagaggaggacctgtga

SEQ ID NO: 237 - protein, 254 aa, Artificial Sequence
  Synthetic Construct
MQSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVSSGGGGSGGGGSGGGGSAQVLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVKGSGEQKLISEEDL

SEQ ID NO: 238 - DNA, 765 bp, Artificial Sequence; identical to SEQ ID NO: 226
  Synthetic Construct

SEQ ID NO: 239 - protein, 254 aa, Artificial Sequence; identical to SEQ ID NO: 227
  Synthetic Construct

SEQ ID NO: 240-241 - skipped (2 entries)

SEQ ID NO: 242 - DNA, 787 bp, Artificial Sequence
  Synthetic Construct
atggatgtgctcatgacacaaacacctttgagccttcccgtgacactcggcgaccaggccagtatctcctgcagaagttcccagtctatcgtgcactcaaacggtaatacgtacttggagtggtatctgcaaaaacccggacagtccccaaagctgttgatatacaaggtttctaatcgcttcagcggtgtaccagacagattttctggctccggatcaggaaccgactttacccttaagatttcaagggtggaagccgaggaccttggagtgtactattgcttccaatcaagtcacgtcccatggactgggggcggcaccaagctcgagatcaagggctctacaggaggtggcggatcaggtaaacctggttctggcgaaggaggtggcggttcagatgtgaagttggttgaaagtggaggcggtctcgttaaactgggcggaagtctgaaactttcatgtgcagcttccgggttcaccttctctagctattacatgagttgggtacggcagacccccgaaaagcgactggagctggtggctaccataaattctaacggaggatctacgtattaccccgacaccgtgaaggggcgcatcaccatttctcgggacaatgccaaaaatacactgcagttgcagatgtcatcactgcgaagcgaagacaccgcgctgtactactgtgtaagacagaattacgagggcgcatattggggacagggcactctggtcaccgtgtcagctggcagtggagagcagaagctgatctcagaggaggacctgtgat

SEQ ID NO: 243 - protein, 261 aa, Artificial Sequence
  Synthetic Construct
MDVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTGGGTKLEIKGSTGGGGSGKPGSGEGGGGSDVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSAGSGEQKLISEEDL

SEQ ID NO: 244 - DNA, 786 bp, Artificial Sequence
  Synthetic Construct
atggcaatcgtgatgacccagactcctagctccaaaagcgtgcccgtgggtggaactgttacgatcaactgtcaggcgagcgagtccgtgtacaacaacaaccatctttcttggtatcagcagaagtctggacagccgcccaagctgctcatatacgaagcgtcaaagctggaatccggagtgccacctcgctttaagggaagcgggagtggcacccagttcactctgaccataagtgacgttgtttgtgacgacgctgccacatattattgcagtggttataagcgagtcaccacggacggcatcgcctttggaggtggcaccgaggtggttgtgaagggctctacaggaggtggcggatcaggtaaacctggttctggcgaaggaggtggcggttcacagagcgtggaggaatcaggtggaagactcgttactccaggtactccgctgaccttgacttgtaccgtgagtggcttttccctgtcccgctactacatgacctgggtgaggcaggcacctggcaaagggctggaatacattggggtgatcatacctggcggaacaacatattacgccagttgggcaaaaggtcgctttaccatttccaaaacatccactactgtggacctcagaatcacatccccaactacagaagacacagccacatacttctgcgcaggaggaccaaccggcaactctcatttcacactctggggccagggcactctggtcaccgtgtcatctggcagtggagagcagaagctgatctcagaggaggacctgtga

SEQ ID NO: 245 - protein, 261 aa, Artificial Sequence
  Synthetic Construct
MAIVMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVKGSTGGGGSGKPGSGEGGGGSQSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVSSGSGEQKLISEEDL

SEQ ID NO: 246 - DNA, 780 bp, Artificial Sequence
  Synthetic Construct
atggcacaagtgttgacccagactacttcacccgttagtgcagccgtgggaggcaccgttaccattagctgccagtcatcccagtcagtttataataacaataaccttgcctggttccagcagaaaccaggccagccccccaagctcctgatctatagagccagcaatctgccgagcggggttccttccaggttcagagggagtggtagtgggtctcagttcacactcaccatcagcgaggtgcaatgtgatgacgctgctacctactactgccaggggtacttcagcggattcatcacaacctttggaggtggcaccgaggtggttgtgaagggctctacaggaggtggcggatcaggtaaacctggttctggcgaaggaggtggcggttcacagagcctggaggaatcaggtggaagacttgttacaccaggtacgcccctgactctgacatgtactgtgagcggattttccctgagtagctataacatgggatgggtcagacaggcccctggtgagggtctcgagtggatcggtgtgatcggtaccggaggtatcactcactatgcaacctgggcaaaaggacgcgtggccatcagcaggaccagcacaactgtaggcctgcggatgactagcccaacaaccgaagacacagcaacatacttttgtgtccggagcagcggttccgattggtggtttcacatctggggacagggaactctggtcaccgtgtcatctggcagtggagagcagaagctgatctcagaggaggacctgtga

SEQ ID NO: 247 - protein, 259 aa, Artificial Sequence
  Synthetic Construct
MAQVLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVKGSTGGGGSGKPGSGEGGGGSQSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVSSGSGEQKLISEEDL

SEQ ID NO: 248 - DNA, 798 bp, Artificial Sequence
  Synthetic Construct
atggcaatcgtgatgacccagactccttctagtaagagtgtgcctgtgggagggagcgtgacaatcaactgccaggccagcgagtccgtgtatagtaataatcgactctcatggtatcaacagaagcccggccagcctcctaaattgttgatatactatgcaagtaccctcgaaagcggtgttccatccaggtttaaagggagcggctttgggactcacttcactcttacgattagtggagcacaatgtgatgacgcagctacttattattgtgccggttggagaggggctagaaccgatggagtggattttggaggtggcaccgaggtggttgtgaagggctctacaggaggtggcggatcaggtaaacctggttctggcgaaggaggtggcggttcacaggaacaactggaggaatcaggtggagatttggtgaagccggaggggtctctgaccctgacatgtaccgcctccgggttcagtttttcctcaaattacgtcatgtgctgggtcagacaggcgcctggaaaggggctggaatgggtggcatgcatttggtttgctggaattgtagataccacctactatgccacgtgggcgaaaggacggttcaccatcagcaagactagtagcacaactgttaccctccagatgacaagcctgaccgcagctgacactgccacttatttctgcgcgcgcaaccctgttggatcagttaatctgtggggacaaggcactctggtcaccgtgtcatctggcagtggagagcagaagctgatctcagaggaggacctgtga

SEQ ID NO: 249 - protein, 265 aa, Artificial Seqeuence
  Synthetic Construct
MAIVMTQTPSSKSVPVGGSVTINCQASESVYSNNRLSWYQQKPGQPPKLLIYYASTLESGVPSRFKGSGFGTHFTLTISGAQCDDAATYYCAGWRGARTDGVDFGGGTEVVVKGSTGGGGSGKPGSGEGGGGSQEQLEESGGDLVKPEGSLTLTCTASGFSFSSNYVMCWVRQAPGKGLEWVACIWFAGIVDTTYYATWAKGRFTISKTSSTTVTLQMTSLTAADTATYFCARNPVGSVNLWGQGTLVTVSSGSGEQKLISEEDL

SEQ ID NO: 250-251 - skipped (2 entries)

SEQ ID NO: 252 - DNA, 786 bp, Artificial Sequence
  Synthetic Construct
atggatgtgaagttggttgaaagtggaggcggtctcgttaaactgggcggaagtctgaaactttcatgtgcagcttccgggttcaccttctctagctattacatgagttgggtacggcagacccccgaaaagcgactggagctggtggctaccataaattctaacggaggatctacgtattaccccgacaccgtgaaggggcgcatcaccatttctcgggacaatgccaaaaatacactgcagttgcagatgtcatcactgcgaagcgaagacaccgcgctgtactactgtgtaagacagaattacgagggcgcatattggggacagggcactctggtcaccgtgtcagctggctctacaggaggtggcggatcaggtaaacctggttctggcgaaggaggtggcggttcagatgtgctcatgacacaaacacctttgagccttcccgtgacactcggcgaccaggccagtatctcctgcagaagttcccagtctatcgtgcactcaaacggtaatacgtacttggagtggtatctgcaaaaacccggacagtccccaaagctgttgatatacaaggtttctaatcgcttcagcggtgtaccagacagattttctggctccggatcaggaaccgactttacccttaagatttcaagggtggaagccgaggaccttggagtgtactattgcttccaatcaagtcacgtcccatggactgggggcggcaccaagctcgagatcaagggcagtggagagcagaagctgatctcagaggaggacctgtga

SEQ ID NO: 253 - protein, 261 aa, Artificial Sequence
  Synthetic Construct
MDVKLVESGGGLVKLGGSLKLSCAASGFTFSSYYMSWVRQTPEKRLELVATINSNGGSTYYPDTVKGRITISRDNAKNTLQLQMSSLRSEDTALYYCVRQNYEGAYWGQGTLVTVSAGSTGGGGSGKPGSGEGGGGSDVLMTQTPLSLPVTLGDQASISCRSSQSIVHSNGNTYLEWYLQKPGQSPKLLIYKVSNRFSGVPDRFSGSGSGTDFTLKISRVEAEDLGVYYCFQSSHVPWTGGGTKLEIKGSGEQKLISEEDL

SEQ ID NO: 254 - DNA, 786 bp, Artificial Sequence
  Synthetic Construct
atgcagagcgtggaggaatcaggtggaagactcgttactccaggtactccgctgaccttgacttgtaccgtgagtggcttttccctgtcccgctactacatgacctgggtgaggcaggcacctggcaaagggctggaatacattggggtgatcatacctggcggaacaacatattacgccagttgggcaaaaggtcgctttaccatttccaaaacatccactactgtggacctcagaatcacatccccaactacagaagacacagccacatacttctgcgcaggaggaccaaccggcaactctcatttcacactctggggccagggcactctggtcaccgtgtcatctggctctacaggaggtggcggatcaggtaaacctggttctggcgaaggaggtggcggttcagcaatcgtgatgacccagactcctagctccaaaagcgtgcccgtgggtggaactgttacgatcaactgtcaggcgagcgagtccgtgtacaacaacaaccatctttcttggtatcagcagaagtctggacagccgcccaagctgctcatatacgaagcgtcaaagctggaatccggagtgccacctcgctttaagggaagcgggagtggcacccagttcactctgaccataagtgacgttgtttgtgacgacgctgccacatattattgcagtggttataagcgagtcaccacggacggcatcgcctttggaggtggcaccgaggtggttgtgaagggcagtggagagcagaagctgatctcagaggaggacctgtga

SEQ ID NO: 255 - protein, 261 aa, Artificial Sequence
  Synthetic Construct
MQSVEESGGRLVTPGTPLTLTCTVSGFSLSRYYMTWVRQAPGKGLEYIGVIIPGGTTYYASWAKGRFTISKTSTTVDLRITSPTTEDTATYFCAGGPTGNSHFTLWGQGTLVTVSSGSTGGGGSGKPGSGEGGGGSAIVMTQTPSSKSVPVGGTVTINCQASESVYNNNHLSWYQQKSGQPPKLLIYEASKLESGVPPRFKGSGSGTQFTLTISDVVCDDAATYYCSGYKRVTTDGIAFGGGTEVVVKGSGEQKLISEEDL

SEQ ID NO: 256 - DNA, 780 bp, Artificial Sequence
  Synthetic Construct
atgcagagcctggaggaatcaggtggaagacttgttacaccaggtacgcccctgactctgacatgtactgtgagcggattttccctgagtagctataacatgggatgggtcagacaggcccctggtgagggtctcgagtggatcggtgtgatcggtaccggaggtatcactcactatgcaacctgggcaaaaggacgcgtggccatcagcaggaccagcacaactgtaggcctgcggatgactagcccaacaaccgaagacacagcaacatacttttgtgtccggagcagcggttccgattggtggtttcacatctggggacagggaactctggtcaccgtgtcatctggctctacaggaggtggcggatcaggtaaacctggttctggcgaaggaggtggcggttcagcacaagtgttgacccagactacttcacccgttagtgcagccgtgggaggcaccgttaccattagctgccagtcatcccagtcagtttataataacaataaccttgcctggttccagcagaaaccaggccagccccccaagctcctgatctatagagccagcaatctgccgagcggggttccttccaggttcagagggagtggtagtgggtctcagttcacactcaccatcagcgaggtgcaatgtgatgacgctgctacctactactgccaggggtacttcagcggattcatcacaacctttggaggtggcaccgaggtggttgtgaagggcagtggagagcagaagctgatctcagaggaggacctgtga

SEQ ID NO: 257 - protein, 259 aa, Artificial Sequence
  Synthetic Construct
MQSLEESGGRLVTPGTPLTLTCTVSGFSLSSYNMGWVRQAPGEGLEWIGVIGTGGITHYATWAKGRVAISRTSTTVGLRMTSPTTEDTATYFCVRSSGSDWWFHIWGQGTLVTVSSGSTGGGGSGKPGSGEGGGGSAQVLTQTTSPVSAAVGGTVTISCQSSQSVYNNNNLAWFQQKPGQPPKLLIYRASNLPSGVPSRFRGSGSGSQFTLTISEVQCDDAATYYCQGYFSGFITTFGGGTEVVVKGSGEQKLISEEDL

SEQ ID NO: 258 - DNA, 798 bp, Artificial Sequence
  Synthetic Construct
atgcaggaacaactggaggaatcaggtggagatttggtgaagccggaggggtctctgaccctgacatgtaccgcctccgggttcagtttttcctcaaattacgtcatgtgctgggtcagacaggcgcctggaaaggggctggaatgggtggcatgcatttggtttgctggaattgtagataccacctactatgccacgtgggcgaaaggacggttcaccatcagcaagactagtagcacaactgttaccctccagatgacaagcctgaccgcagctgacactgccacttatttctgcgcgcgcaaccctgttggatcagttaatctgtggggacaaggcactctggtcaccgtgtcatctggctctacaggaggtggcggatcaggtaaacctggttctggcgaaggaggtggcggttcagcaatcgtgatgacccagactccttctagtaagagtgtgcctgtgggagggagcgtgacaatcaactgccaggccagcgagtccgtgtatagtaataatcgactctcatggtatcaacagaagcccggccagcctcctaaattgttgatatactatgcaagtaccctcgaaagcggtgttccatccaggtttaaagggagcggctttgggactcacttcactcttacgattagtggagcacaatgtgatgacgcagctacttattattgtgccggttggagaggggctagaaccgatggagtggattttggaggtggcaccgaggtggttgtgaagggcagtggagagcagaagctgatctcagaggaggacctgtga

SEQ ID NO: 259 - protein, 265 aa, Artificial Sequence
  Synthetic Construct
MQEQLEESGGDLVKPEGSLTLTCTASGFSFSSNYVMCWVRQAPGKGLEWVACIWFAGIVDTTYYATWAKGRFTISKTSSTTVTLQMTSLTAADTATYFCARNPVGSVNLWGQGTLVTVSSGSTGGGGSGKPGSGEGGGGSAIVMTQTPSSKSVPVGGSVTINCQASESVYSNNRLSWYQQKPGQPPKLLIYYASTLESGVPSRFKGSGFGTHFTLTISGAQCDDAATYYCAGWRGARTDGVDFGGGTEVVVKGSGEQKLISEEDL